(19) United States
(12) Patent Application Publication
(10) Pub. No.: US 2009/0263397 A1
(43) Pub. Date: Oct. 22, 2009

(54) Combination anti-cancer therapy

(76) Inventors: Elizabeth A. Buck, Huntington, NY (US); Alexandra Eyzaguirre, Bayside, NY (US); David M. Epstein, Huntington, NY (US); Mark R. Miglarese, Superior, CO (US)

Correspondence Address: OSI PHARMACEUTICALS, INC., 41 PINELAWN ROAD, MELVILLE, NY, 11747, US

(21) Appl. No.: 12/217,594

(22) Filed: Jul. 7, 2008

Related U.S. Application Data
(60) Provisional application No. 60/958,521, filed on Jul. 6, 2007.
(60) Provisional application No. 61/068,611, filed on Mar. 7, 2008.

(51) Int. Cl. A61K 31/4985; A61P 35/00; A61K 31/704; A61K 33/24; A61K 31/4745; A61K 31/7048; A61K 31/337; A61K 31/436; A61K 39/395; G01N 33/53

(52) U.S. Cl. 424/141.1; 514/34; 424/649; 514/283; 514/27; 514/449; 514/291; 514/249; 435/4

(57) ABSTRACT

The present invention provides a method for treating tumors or tumor metastases in a patient, comprising administering to said patient simultaneously or sequentially a therapeutically effective amount of a combination of an anti-cancer agent or treatment that elevates pAkt levels in tumor cells and an IGF-1R kinase inhibitor of Formula (I) (e.g. OSI-906). Examples of such anti-cancer agents or treatments include doxorubicin, cisplatin, and ionizing radiation. The present invention also provides a pharmaceutical composition comprising an anti-cancer agent that elevates pAkt levels in tumor cells and an IGF-1R kinase inhibitor of Formula (I), in a pharmaceutically acceptable carrier. The present invention also provides a method of identifying tumor cells that will respond most favorably to treatment with a combination of an anti-cancer agent or treatment that elevates pAkt levels in tumor cells and an IGF-1R kinase inhibitor.

### CROSS-REFERENCE TO RELATED APPLICATIONS

This application claims the benefit of U.S. Provisional Application No. 60/958,521, filed Jul. 6, 2007, and U.S. Provisional Application No. 61/068,611, filed Mar. 7, 2008, both of which are herein incorporated by reference in their entirety.

### BACKGROUND OF THE INVENTION

The present invention is directed to compositions and methods for treating cancer patients. Cancer is a generic name for a wide range of cellular malignancies characterized by unregulated growth, lack of differentiation, and the ability to invade local tissues and metastasize. These neoplastic malignancies affect, with various degrees of prevalence, every tissue and organ in the body.

A multitude of therapeutic agents have been developed over the past few decades for the treatment of various types of cancer. The most commonly used types of anticancer agents include: DNA-alkylating agents (e.g., cyclophosphamide, ifosfamide), antimetabolites (e.g., methotrexate, a folate antagonist, and 5-fluorouracil, a pyrimidine antagonist), microtubule disrupters (e.g., vincristine, vinblastine, paclitaxel), DNA intercalators (e.g., doxorubicin, daunomycin, cisplatin), and hormone therapy (e.g., tamoxifen, flutamide). More recently, gene targeted therapies, such as protein-tyrosine kinase inhibitors (e.g. imatinib; the EGFR kinase inhibitor, erlotinib) have increasingly been used in cancer therapy.

An anti-neoplastic drug would ideally kill cancer cells selectively, with a wide therapeutic index relative to its toxicity towards non-malignant cells. It would also retain its efficacy against malignant cells, even after prolonged exposure to the drug. Unfortunately, none of the current chemotherapies possess such an ideal profile. Instead, most possess very narrow therapeutic indexes. Furthermore, cancerous cells exposed to slightly sub-lethal concentrations of a chemotherapeutic agent will very often develop resistance to such an agent, and quite often cross-resistance to several other antineoplastic agents as well. Additionally, for any given cancer type one frequently cannot predict which patient is likely to respond to a particular treatment, even with newer gene-targeted therapies, such as EGFR kinase inhibitors, thus necessitating considerable trial and error, often at considerable risk and discomfort to the patient, in order to find the most effective therapy.

Thus, there is a need for more efficacious treatment for neoplasia and other proliferative disorders, and for more effective means for determining which tumors will respond to which treatment. Strategies for enhancing the therapeutic efficacy of existing drugs have involved changes in the schedule for their administration, and also their use in combination with other anticancer or biochemical modulating agents. Combination therapy is well known as a method that can result in greater efficacy and diminished side effects relative to the use of the therapeutically relevant dose of each agent alone. In some cases, the efficacy of the drug combination is additive (the efficacy of the combination is approximately equal to the sum of the effects of each drug alone), but in other cases the effect is synergistic (the efficacy of the combination is greater than the sum of the effects of each drug given alone).

Several anti-cancer agents and treatments exert their anti-cancer effects by promoting tumor cell apoptosis. However, this effect is frequently limited by the fact that these agents can cause activation of Akt (and elevated pAkt levels), which stimulates pro-survival, anti-apoptotic pathways in the tumor cells (e.g. West, K. A. et al. (2002) Drug Resistance Updates 5(6):234-248; Clark, A. S. et al. (2002) Molec. Cancer Therapeutics 1:707-717; Brognard, J. et al. (2001) Cancer Res. 61:3986-3997; Kim, T-J. et al. (2006) Brit. J. Cancer 94:1678-1682; Gupta, A. K. et al (2002) Clin. Cancer Res. 8:885-892; Kim, I-A. et al. (2005) Cancer Res. 65(17):7902-7910; Li, X. et al. (2005) Breast Cancer Res. 7(5):R589-R597; VanderWeele, D. J. et al. (2004) Mol. Cancer. Ther. 3:1605-1613; Han, E. K-H, et al. (2007) Oncogene doi: 10.1038/sj.onc.1210343). Several agents have been reported that potentiate the pro-apoptotic affects of such anti-cancer agents and treatments, such as inhibitors of IGF-1R, mTOR, or Akt (e.g. Wendel, H-G. et al. (2004) Nature 428:332-337; Shi, Y. et al. (1995) Cancer Res. 55:1982-1988; Beuvink, I. et al. (2005) Cell 120:747-759; Mungamuri, S. K. et al. (2006) Cancer Res. 66(9):4715-4724; Wu, C. et al. (2005) Molecular Cancer 4(25) doi:10.1186/1476-4598-4-25; Smolewski, P. (2006) Expert Opin. Investig. Drugs 15(10):1201-1227; Mondesire, W. H. et al. (2004) Clin Cancer Res. 10:7031-7042; Shi, Y. et al. (2005) Neoplasia 7(11):992-1000; Jerome, L. (2003) Endocrine-Related Cancer 10:561-578; Krystal, G. et al. (2002) Mol. Cancer. Ther. 1:913-922; Goetsch, L. et al. (2005) Int. J. Cancer 113:316-328; Gupta, A. K. et al. (2005) Cancer Res. 65(18):8256-8265; Min, Y. et al. (2005) Gut 54:591-600; Fujita, N. et al (2003) Cancer Chemother. Pharmacol. 52(Suppl.1):S24-S28; US Published Patent Application No. 2004/0209930; Huang, G. S. et al. (2007) AACR Annual Meeting Proceedings, Abstract No. 4748; Westfall, S. D. et al. (2005) Mol. Cancer. Ther. 4(11):1764-1771). However, such agents have also been reported to only produce additive affects in combination with such anticancer agents or treatments (Mondesire, W. H. et al. (2004) Clin Cancer res. 10:7031-7042; Hopfner, M. et al. (2006) Endocrine-Related Cancer 13:135-149; Baradari, V. et al. (2005) Z Gastroenterol. 43 DOI: 10.1055/s-2005-920141; Rivera, V. M. et al. (2004) Proc. Amer. Assoc. Cancer Res. 45 (Abs 3887)). The invention described herein provides new anti-cancer combination therapies that utilize a new class of IGF-1R kinase inhibitor to potentiate the pro-apoptotic affects of such anti-cancer agents and treatments. These new IGF-1R kinase inhibitors are relatively specific, orally-available, small-molecule compounds.

IGF-1R is a transmembrane RTK that binds primarily to IGF-1 but also to IGF-II and insulin with lower affinity. Binding of IGF-1 to its receptor results in receptor oligomerization, activation of tyrosine kinase, intermolecular receptor autophosphorylation and phosphorylation of cellular substrates (major substrates are IRS1 and Shc). The ligand-activated IGF-1R induces mitogenic activity in normal cells and plays an important role in abnormal growth. A major physiological role of the IGF-1 system is the promotion of normal growth and regeneration. Overexpressed IGF-1R (type 1 insulin-like growth factor receptor) can initiate mitogenesis and promote ligand-dependent neoplastic transformation. Furthermore, IGF-1R plays an important role in the establishment and maintenance of the malignant phenotype. Unlike the epidermal growth factor (EGF) receptor, no mutant oncogenic forms of the IGF-1R have been identified. However, several oncogenes have been demonstrated to affect IGF-1 and IGF-1R expression. The correlation between a reduction of IGF-1R expression and resistance to transformation has been seen. Exposure of cells to the mRNA antisense to IGF-1R RNA prevents soft agar growth of several human tumor cell lines. IGF-1R abrogates progression into apoptosis, both in vivo and in vitro. It has also been shown that a decrease in the level of IGF-1R below wild-type levels causes apoptosis of tumor cells in vivo. The ability of IGF-1R disruption to cause apoptosis appears to be diminished in normal, non-tumorigenic cells.

The IGF-1 pathway in human tumor development has an important role. IGF-1R overexpression is frequently found in various tumors (breast, colon, lung, sarcoma) and is often associated with an aggressive phenotype. High circulating IGF1 concentrations are strongly correlated with prostate, lung and breast cancer risk. Furthermore, IGF-1R is required for establishment and maintenance of the transformed phenotype in vitro and in vivo (Baserga R. Exp. Cell. Res., 1999, 253, 1-6). The kinase activity of IGF-1R is essential for the transforming activity of several oncogenes: EGFR, PDGFR, SV40 T antigen, activated Ras, Raf, and v-Src. The expression of IGF-1R in normal fibroblasts induces neoplastic phenotypes, which can then form tumors in vivo. IGF-1R expression plays an important role in anchorage-independent growth. IGF-1R has also been shown to protect cells from chemotherapy-, radiation-, and cytokine-induced apoptosis. Conversely, inhibition of endogenous IGF-1R by dominant negative IGF-1R, triple helix formation or antisense expression vector has been shown to repress transforming activity in vitro and tumor growth in animal models.

It has been recognized that inhibitors of protein-tyrosine kinases are useful as selective inhibitors of the growth of mammalian cancer cells. For example, Gleevec™ (also known as imatinib mesylate), a 2-phenylpyrimidine tyrosine kinase inhibitor that inhibits the kinase activity of the BCR-ABL fusion gene product, has been approved by the U.S. Food and Drug Administration for the treatment of CML. The 4-anilinoquinazoline compound Tarceva™ (erlotinib HCl) has also been recently approved by the FDA, and selectively inhibits EGF receptor kinase with high potency. The development for use as anti-tumor agents of compounds that directly inhibit the kinase activity of IGF-1R, as well as antibodies that reduce IGF-1R kinase activity by blocking IGF-1R activation or antisense oligonucleotides that block IGF-1R expression, are areas of intense research effort (e.g. see Larsson, O. et al (2005) Brit. J. Cancer 92:2097-2101; Ibrahim, Y. H. and Yee, D. (2005) Clin. Cancer Res. 11:944s-950s; Mitsiades, C. S. et al. (2004) Cancer Cell 5:221-230; Camirand, A. et al. (2005) Breast Cancer Research 7:R570-R579 (DOI 10.1186/bcr1028); Camirand, A. and Pollak, M. (2004) Brit. J. Cancer 90:1825-1829; Garcia-Echeverria, C. et al. (2004) Cancer Cell 5:231-239).

### SUMMARY OF THE INVENTION

The present invention provides a method for treating tumors or tumor metastases in a patient, comprising administering to said patient simultaneously or sequentially a therapeutically effective amount of a combination of an anti-cancer agent or treatment that elevates pAkt levels in tumor cells and an IGF-1R kinase inhibitor of Formula (I).

In any of the methods, compositions or kits of the invention described herein, an anti-cancer agent or treatment that elevates pAkt levels in tumor cells can be any anti-cancer agent or treatment presently known or yet to be characterized that elevates pAkt levels in tumor cells. In one embodiment, the anti-cancer agent or treatment that elevates pAkt levels is a chemotherapeutic agent. Examples of such chemotherapeutic agents that elevate pAkt levels include anthracyclins, such as doxorubicin or daunorubicin; tamoxifen; DNA-damaging agents, such as cisplatin or carboplatin; topoisomerase inhibitors, such as camptothecin or etoposide; and microtubule-directed agents, such as vincristine, colchicines, vinblastine, decetaxel, and paclitaxel. In another embodiment, the anti-cancer agent or treatment that elevates pAkt levels is a form of ionizing radiation. In an other embodiment, the anti-cancer agent or treatment that elevates pAkt levels is a gene-targeted anti-cancer agent. Examples of such gene-targeted anti-cancer agents that elevate pAkt levels include rapamycin; rapalogs (i.e. rapamycin analogs), such as CCI-779 or RAD001; trastuzumab; and the pan-Akt inhibitor A443654.

In any of the methods, compositions or kits of the invention described herein, the IGF-1R kinase inhibitor of Formula (I) can be any IGF-1R kinase inhibitor compound encompassed by Formula (I) that inhibits IGF-1R kinase upon administration to a patient. Specific examples of such inhibitors have been published in US Published Patent Application US 2006/0235031, which is incorporated herein in its entirety, and includes Compound D (OSI-906) as used in the experiments described herein.

An IGF-1R kinase inhibitor of Formula (I) is represented by the formula:

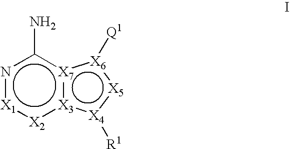

or a pharmaceutically acceptable salt thereof, wherein:
X₁, and X₂ are each independently N or C-(E¹)ₐₐ;
X₅ is N, C-(E¹)ₐₐ, or N-(E¹)ₐₐ;
X₃, X₄, X₆, and X₇ are each independently N or C;
   wherein at least one of X₃, X₄, X₅, X₆, and X₇ is independently N or N-(E¹)ₐₐ;
Q¹ is

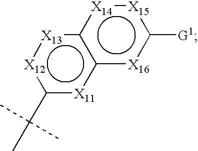

X₁₁, X₁₂, X₁₃, X₁₄, X₁₅, and X₁₆ are each independently N, C-(E¹¹)_{bb}, or N⁺—O⁻;
wherein at least one of X₁₁, X₁₂, X₁₃, X₁₄, X₁₅, and X₁₆ is N or N⁺—O⁻;
R¹ is absent, C₀₋₁₀alkyl, cycloC₃₋₁₀alkyl, bicycloC₅₋₁₀alkyl, aryl, heteroaryl, aralkyl, heteroaralkyl, heterocyclyl, heterobicycloC₅₋₁₀alkyl, spiroalkyl, or heterospiroalkyl, any of which is optionally substituted by one or more independent G¹¹ substituents;
E¹, E¹¹, G¹, and G⁴¹ are each independently halo, —CF₃, —OCF₃, —OR², —NR²R³(R^{2a})ⱼ₁, —C(═O)R², —CO₂R², —CONR²R³, —NO₂, —CN, —S(O)ⱼ₁R², —SO₂NR²R³, —NR²C(═O)R³, —NR²C(═O)OR³, —NR²C(═O)NR³R^{2a}, —NR²S(O)ⱼ₁R³, —C(═S)OR², —C(═O)SR², —NR₂C(═NR³)NR^{2a}R^{3a}, —NR²C(═NR³)OR^{2a}, NR²C(═NR³)SR^{2a}, —OC(═O)OR², —OC(═O)NR²R³, —OC(═O)SR², —SC(═O)OR², —SC(═O)NR²R³, C₀₋₁₀alkyl, C₂₋₁₀alkenyl, C₂₋₁₀alkynyl, C₁₋₁₀alkoxyC₁₋₁₀alkyl, C₁₋₁₀alkoxyC₂₋₁₀alkenyl, C₁₋₁₀alkoxyC₂₋₁₀alkynyl, C₁₋₁₀alkylthioC₁₋₁₀alkyl, C₁₋₁₀alkylthioC₂₋₁₀alkenyl, C₁₋₁₀alkylthioC₂₋₁₀alkynyl, cycloC₃₋₈alkyl, cycloC₃₋₈alkenyl, cycloC₃₋₈alkylC₁₋₁₀alkyl, cycloC₃₋₈alkenylC₁₋₁₀alkyl, cycloC₃₋₈alkylC₂₋₁₀alkenyl, cycloC₃₋₈alkenylC₂₋₁₀alkenyl, cycloC₃₋₈alkylC₂₋₁₀alkynyl, cycloC₃₋₈alkenylC₂₋₁₀alkynyl, heterocyclyl-C₀₋₁₀alkyl, heterocyclyl-C₂₋₁₀alkenyl, or heterocyclyl-C₂₋₁₀alkynyl, any of which is optionally substituted with one or more independent halo, oxo, —CF₃, —OCF₃, —OR²²², —NR²²²R³³³(R^{222a})ⱼₗₐ, —C(═O)R²²², —CO₂R²²², —C(═O)NR²²²R³³³, —NO₂, —CN, —S(═O)ⱼₗₐR²²², —SO₂NR²²²R³³³, —NR²²²C(═O)R³³³, —NR²²²C(═O)OR³³³, NR²²²C(═O)NR³³³R^{222a}, —NR²²²S(O)ⱼₗₐR³³³, —C(═S)OR²²², —C(═O)SR²²², —NR²²²C(═NR³³³)NR^{222a}R^{333a}, —NR²²²C(═NR³³³)OR^{222a}, —NR²²²C(═NR³³³)SR^{222a}, —OC(═O)OR²²², —OC(═O)NR²²²R³³³, —OC(═O)SR²²², —SC(═O)OR²²², or —SC(═O)NR²²²R³³³ substituents;
or E¹, E¹¹, or G¹ optionally is —(W¹)ₙ—(Y¹)ₘ—R⁴;
or E¹, E¹¹, G¹, or G⁴¹ optionally independently is aryl-C₀₋₁₀alkyl, aryl-C₂₋₁₀alkenyl, aryl-C₂₋₁₀alkynyl, hetaryl-C₀₋₁₀alkyl, hetaryl-C₂₋₁₀alkenyl, or hetaryl-C₂₋₁₀alkynyl, any of which is optionally substituted with one or more independent halo, —CF₃, —OCF₃, —OR²²², —NR²²²R³³³(R^{222a})ⱼ₂ₐ, —C(O)R²²², —CO₂R²²², —C(═O)NR²²²R³³³, —NO₂, —CN, —S(O)ⱼ₂ₐR²²², —SO₂NR²²²R³³³, —NR²²²C(═O)R³³³, —NR²²²C(═O)OR³³³, —NR²²²C(═O)NR³³³R^{222a}, —NR²²²S(O)ⱼ₂ₐR³³³, —C(═S)OR²²², —C(═O)SR²²², —NR²²²C(═NR³³³)NR^{222a}R^{333a}, —NR²²²C(═NR³³³)OR^{222a}, —NR²²²C(═NR³³³)SR^{222a}, —OC(═O)OR²²², —OC(═O)NR²²²R³³³, —OC(═O)SR²²², —SC(═O)OR²²², or —SC(═O)NR²²²R³³³ substituents;
G¹¹ is halo, oxo, —CF₃, —OCF₃, —OR²¹, —NR²¹R³¹(R^{2a1})ⱼ₄, —C(O)R²¹, —CO₂R²¹, —C(═O)NR²¹R³¹, —NO₂, —CN, —S(O)ⱼ₄R²¹, —SO₂NR²¹R³¹, NR²¹(C═O)R³¹, NR²¹C(═O)OR³¹, NR²¹C(═O)NR³¹R^{2a1}, NR²¹S(O)ⱼ₄R³¹, —C(═S)OR²¹, —C(═O)SR²¹, —NR²¹C(═NR³¹)NR^{2a1}R^{3a1}, —NR²¹C(═NR³¹)OR^{2a1}, —NR²¹C(═NR³¹)SR^{2a1}, —OC(═O)OR²¹, —OC(═O)NR²¹R³¹, —OC(═O)SR²¹, —SC(═O)OR²¹, —SC(═O)NR²¹R³¹, —P(O)OR²¹OR³¹, C₁₋₁₀alkylidene, C₀₋₁₀alkyl, C₂₋₁₀alkenyl, C₂₋₁₀alkynyl, C₁₋₁₀alkoxyC₁₋₁₀alkyl, C₁₋₁₀alkoxyC₂₋₁₀alkenyl, C₁₋₁₀alkoxyC₂₋₁₀alkynyl, C₁₋₁₀alkylthioC₁₋₁₀alkyl, C₁₋₁₀alkylthioC₂₋₁₀alkenyl, C₁₋₁₀alkylthioC₂₋₁₀alkynyl, cycloC₃₋₈alkyl, cycloC₃₋₈alkenyl, cycloC₃₋₈alkylC₁₋₁₀alkyl, cycloC₃₋₈alkenylC₁₋₁₀alkyl, cycloC₃₋₈alkylC₂₋₁₀alkenyl, cycloC₃₋₈alkenylC₂₋₁₀alkenyl, cycloC₃₋₈alkylC₂₋₁₀alkynyl, cycloC₃₋₈alkenylC₂₋₁₀alkynyl, heterocyclyl-C₀₋₁₀alkyl, heterocyclyl-C₂₋₁₀alkenyl, or heterocyclyl-C₂₋₁₀alkynyl, any of which is optionally substituted with one or more independent halo, oxo, —CF₃, —OCF₃, —OR²²²¹, —NR²²²¹R³³³¹(R^{222a1})ⱼ₄ₐ, —C(O)R²²²¹, —CO₂R²²²¹, —C(═O)NR²²²¹R³³³¹, —NO₂, —CN, —S(O)ⱼ₄ₐR²²²¹, —SO₂NR²²²¹R³³³¹, —NR²²²¹C(═O)R³³³¹, —NR²²²¹C(═O)OR³³³¹, —NR²²²¹C(═O)NR³³³¹R^{222a1}, —NR²²²¹S(O)ⱼ₄ₐR³³³¹, —C(═S)OR²²²¹, —C(═O)SR²²²¹, —NR²²²¹C(═NR³³³¹)NR^{222a1}R^{333a1}, —NR²²²¹C(═NR³³³¹)OR^{222a1}, —NR²²²¹C(═NR^{333l})SR^{222a1}, —OC(═O)OR²²²¹, —OC(═O)NR²²²¹R³³³¹, —OC(═O)SR²²²¹, —SC(═O)OR²²²¹, —P(O)OR²²²¹OR³³³¹, or —SC(═O)NR²²²¹R³³³¹ substituents;
or G¹¹ is aryl-C₀₋₁₀alkyl, aryl-C₂₋₁₀ alkenyl, aryl-C₂₋₁₀alkynyl, hetaryl-C₀₋₁₀alkyl, hetaryl-C₂₋₁₀alkenyl, or hetaryl-C₂₋₁₀alkynyl, any of which is optionally substituted with one or more independent halo, —CF₃, —OCF₃, —OR²²²¹, —NR²²²¹R³³³¹(R^{222a1})ⱼ₅ₐ, —C(O)R²²²¹, —CO₂R²²²¹, —C(═O)NR²²²¹R³³³¹, —NO₂, —CN, —S(O)ⱼ₅ₐR²²²¹, —SO₂NR²²²¹R³³³¹, —NR²²²¹C(═O)R³³³¹, —NR²²²¹C(═O)OR³³³¹, NR²²²¹C(═O)NR³³³¹R^{222a1}, —NR²²²¹S(O)ⱼ₅ₐR³³³¹, —C(═S)OR²²²¹, —C(═O)SR²²²¹, —NR²²²¹C(═NR³³³¹)NR^{222a1}R^{333a1}, —NR²²²¹C(═NR³³³¹)OR^{222a1}, —NR²²²¹C(═NR³³³¹)SR^{222a1}, —OC(═O)OR²²²¹, —OC(═O)NR²²²¹R³³³¹, —OC(═O)SR²²²¹, —SC(═O)OR²²²¹, —P(O)OR²²²¹R³³³¹, or —SC(═O)NR²²²¹R³³³¹ substituents;
or G¹¹ is C, taken together with the carbon to which it is attached forms a C═C double bond which is substituted with R⁵ and G¹¹¹;
R²R^{2a}, R³, R^{3a}, R²²²R^{222a}, R³³³, R^{333a}, R²¹, R^{2a1}, R³¹, R^{3a1}, R²²²¹R^{222a1}, R³³³¹, and R^{333a1} are each independently C₀₋₁₀alkyl, C₂₋₁₀alkenyl, C₂₋₁₀alkynyl, C₁₋₁₀alkoxyC₁₋₁₀alkyl, C₁₋₁₀alkoxyC₂₋₁₀alkenyl, C₁₋₁₀alkoxyC₂₋₁₀alkynyl, C₁₋₁₀alkylthioC₁₋₁₀alkyl, C₁₋₁₀alkylthioC₂₋₁₀alkenyl, C₁₋₁₀alkylthioC₂₋₁₀alkynyl, cycloC₃₋₈alkyl, cycloC₃₋₈alkenyl, cycloC₃₋₈alkylC₁₋₁₀alkyl, cycloC₃₋₈alkenylC₁₋₁₀alkyl, cycloC₃₋₈alkylC₂₋₁₀alkenyl, cycloC₃₋₈alkenylC₂₋₁₀alkenyl, cycloC₃₋₈alkylC₂₋₁₀ alkynyl, cycloC₃₋₈alkenylC₂₋₁₀alkynyl, heterocyclyl-C₀₋₁₀alkyl, heterocyclyl-C₂₋₁₀alkenyl, heterocyclyl-C₂₋₁₀alkynyl, aryl-C₀₋₁₀alkyl, aryl-C₂₋₁₀alkenyl, or aryl-C₂₋₁₀ alkynyl, hetaryl-C₀₋₁₀alkyl, hetaryl-C₂₋₁₀alkenyl, or hetaryl-C₂₋₁₀alkynyl, any of which is optionally substituted by one or more independent G¹¹¹ substituents;
or in the case of —NR²R³(R^{2a})ⱼ₁ or —NR²²²R³³³(R^{222a})ⱼₐ or —NR²²²R³³³(R^{222a})ⱼ₂ₐ or —NR²¹R³¹(R^{2a1})ⱼ₄ or —NR²²²¹R³³³¹(R^{222a1})ⱼ₄ₐ or —NR²²²¹R³³³¹(R^{222a1})ⱼ₅ₐ, then R² and R³, or R²²² and R³³³, or R²²²¹ and R³³³¹, respectfully, are optionally taken together with the nitrogen atom to which they are attached to form a 3-10 membered saturated or unsaturated ring, wherein said ring is optionally substituted by one or more independent G¹¹¹¹ substituents and wherein said ring optionally includes one or more heteroatoms other than the nitrogen to which R² and R³, or R²²² and R³³³, or R²²²¹ and R³³³¹ are attached;
W¹ and Y¹ are each independently —O—, —NR⁷—, —S(O)ⱼ₇—, —CR⁵R⁶—, —N(C(O)OR⁷)—, —N(C(O)R⁷)—, —N(SO₂R⁷)—, —CH₂O—, —CH₂S—, —CH₂N(R⁷)—, —CH(NR⁷)—, —CH₂N(C(O)R⁷)—, —CH₂N(C(O)OR⁷)—, —CH₂N(SO₂R⁷)—, —CH(NHR⁷)—, —CH(NHC(O)R⁷)—, —CH(NHSO₂R⁷)—, —CH(NHC(O)OR⁷)—, —CH(OC(O)R⁷)—, —CH(OC(O)NHR⁷)—, —CH═CH—, —C≡C—, —C(═NOR⁷)—, —C(O)—, —CH(OR⁷)—, —C(O)N(R⁷)—, —N(R⁷)C(O)—, —N(R⁷)S(O)—, —N(R⁷)S(O)₂—, —OC(O)N(R⁷)—, —N(R⁷)C(O)N(R⁸)—, —NR⁷C(O)O—, —S(O)N(R⁷)—, —S(O)₂N(R⁷)—, —N(C(O)R⁷)S(O)—, —N(C(O)R⁷)S(O)₂—, —N(R⁷)S(O)N(R⁸)—, —N(R⁷)S(O)₂N(R⁸)—, —C(O)N(R⁷)C(O)—, —S(O)N(R⁷)C(O)—, —S(O)₂N(R⁷)C(O)—, —OS(O)N(R⁷)—, —OS(O)₂N(R⁷)—, —N(R⁷)S(O)O—, —N(R⁷)S(O)₂O—, —N(R⁷)S(O)C(O)—, —N(R⁷)S(O)₂C(O)—, —SON(C(O)R⁷)—, —SO₂N(C(O)R⁷)—, —N(R⁷)SON(R⁸)—, —N(R⁷)SO₂N(R⁸)—, —C(O)O—, —N(R⁷)P(OR⁸)O—, —N(R⁷)P(OR⁸)—, —N(R⁷)P(O)(OR⁸)O—, —N(R⁷)P(O)(OR⁸)—, —N(C(O)R⁷)P(OR⁸)O—, —N(C(O)R⁷)P(OR⁸)—, —N(C(O)R⁷)P(O)(OR⁸)O—, —N(C(O)R⁷)P(OR⁸)—, —CH(R⁷)S(O)—, —CH(R⁷)S(O)₂—, —CH(R⁷)N(C(O)OR⁸)—, —CH(R⁷)N(C(O)R⁸)—, —CH(R⁷)N(SO₂R⁸)—, —CH(R⁷)O—, —CH(R⁷)S—, —CH(R⁷)N(R⁸)—, —CH(R⁷)N(C(O)R⁸)—, —CH(R⁷)N(C(O)OR⁸)—, —CH(R⁷)N(SO₂R⁸)—, —CH(R⁷)C(═NOR⁸)—, —CH(R⁷)C(O)—, —CH(R⁷)CH(OR⁸)—, —CH(R⁷)C(O)N(R⁸)—, —CH(R⁷)N(R⁸)C(O)—, —CH(R⁷)N(R⁸)S(O)—, —CH(R⁷)N(R⁸)S(O)₂—, —CH(R⁷)OC(O)N(R⁸)—, —CH(R⁷)N(R⁸)C(O)N(R^{7a})—, —CH(R⁷)NR⁸C(O)O—, —CH(R⁷)S(O)N(R⁸)—, —CH(R⁷)S(O)₂N(R⁸)—, —CH(R⁷)N(C(O)R⁸)S(O)—, —CH(R⁷)N(C(O)R⁸)S(O)—, —CH(R⁷)N(R⁸)S(O)N(R^{7a})—, —CH(R⁷)N(R⁸)S(O)₂N(R^{7a})—, —CH(R⁷)C(O)N(R⁸)C(O)—, —CH(R⁷)S(O)N(R⁸)C(O)—, —CH(R⁷)S(O)₂N(R⁸)C(O)—, —CH(R⁷)OS(O)N(R⁸)—, —CH(R⁷)OS(O)₂N(R⁸)—, —CH(R⁷)N(R⁸)S(O)O—, —CH(R⁷)N(R⁸)S(O)₂O—, —CH(R⁷)N(R⁸)S(O)C(O)—, —CH(R⁷)N(R⁸)S(O)₂C(O)—, —CH(R⁷)SON(C(O)R⁸)—, —CH(R⁷)SO₂N(C(O)R⁸)—, —CH(R⁷)N(R⁸)SON(R^{7a})—, —CH(R⁷)N(R⁸)SO₂N(R^{7a})—, —CH(R⁷)C(O)O—, —CH(R⁷)N(R⁸)P(OR^{7a})O—, —CH(R⁷)N(R⁸)P(OR^{7a})—, —CH(R⁷)N(R⁸)P(O)(OR^{7a})O—, —CH(R⁷)N(R⁸)P(O)(OR^{7a})—, —CH(R⁷)N(C(O)R⁸)P(OR^{7a})O—, —CH(R⁷)N(C(O)R⁸)P(OR^{7a})—, —CH(R⁷)N(C(O)R⁸)P(O)(OR^{7a})O—, or —CH(R⁷)N(C(O)R⁸)P(OR^{7a})—;
R⁵, R⁶, G¹¹¹, and G¹¹¹¹ are each independently C₀₋₁₀alkyl, C₂₋₁₀alkenyl, C₂₋₁₀alkynyl, C₁₋₁₀alkoxyC₁₋₁₀alkyl, C₁₋₁₀alkoxyC₂₋₁₀alkenyl, C₁₋₁₀alkoxyC₂₋₁₀alkynyl, C₁₋₁₀alkylthioC₁₋₁₀alkyl, C₁₋₁₀alkylthioC₂₋₁₀alkenyl, C₁₋₁₀alkylthioC₂₋₁₀alkynyl, cycloC₃₋₈alkyl, cycloC₃₋₈alkenyl, cycloC₃₋₈alkylC₁₋₁₀alkyl, cycloC₃₋₈alkenylC₁₋₁₀alkyl, cycloC₃₋₈alkylC₂₋₁₀alkenyl, cycloC₃₋₈alkenylC₂₋₁₀alkenyl, cycloC₃₋₈alkylC₂₋₁₀alkynyl, cycloC₃₋₈alkenylC₂₋₁₀alkynyl, heterocyclyl-C₀₋₁₀alkyl, heterocyclyl-C₂₋₁₀alkenyl, heterocyclyl-C₂₋₁₀alkynyl, aryl-C₀₋₁₀alkyl, aryl-C₂₋₁₀alkenyl, aryl-C₂₋₁₀alkynyl, hetaryl-C₀₋₁₀alkyl, hetaryl-C₂₋₁₀ alkenyl, or hetaryl-C₂₋₁₀alkynyl, any of which is optionally substituted with one or more independent halo, —CF₃, —OCF₃, —OR⁷⁷, —NR⁷⁷R⁸⁷, —C(O)R⁷⁷, —CO₂R⁷⁷, —CONR⁷⁷R⁸⁷, —NO₂, —CN, —S(O)ⱼ₅ₐR⁷⁷, —SO₂NR⁷⁷R⁸⁷, —NR⁷⁷C(═O)R⁸⁷, NR⁷⁷C(═O)OR⁸⁷, —NR⁷⁷C(═O)NR⁷⁸R⁸⁷, —NR⁷⁷S(O)ⱼ₅ₐR⁸⁷, —C(═S)OR⁷⁷, —C(═O)SR⁷⁷, —NR⁷⁷C(═NR⁸⁷)NR⁷⁸R⁸⁸, —NR⁷⁷C(═NR⁸⁷)OR⁷⁸, —NR⁷⁷C(═NR⁸⁷)SR⁷¹, —OC(═O)OR⁷⁷, —OC(═O)NR⁷⁷R⁸⁷, —OC(═O)SR⁷⁷, —SC(═O)OR⁷⁷, —P(O)OR⁷⁷OR⁸⁷, or —SC(═O)NR⁷⁷R⁸⁷ substituents;
or R⁵ with R⁶ are optionally taken together with the carbon atom to which they are attached to form a 3-10 membered saturated or unsaturated ring, wherein said ring is optionally substituted with one or more independent R⁶⁹ substituents and wherein said ring optionally includes one or more heteroatoms;
R⁷, R^{7a}, and R⁸ are each independently acyl, C₀₋₁₀alkyl, C₂₋₁₀alkenyl, aryl, heteroaryl, heterocyclyl or cycloC₃₋₁₀alkyl, any of which is optionally substituted by one or more independent G¹¹¹ substituents;
R⁴ is C₀₋₁₀alkyl, C₂₋₁₀alkenyl, C₂₋₁₀alkynyl, aryl, heteroaryl, cycloC₃₋₁₀alkyl, heterocyclyl, cycloC₃₋₈alkenyl, or heterocycloalkenyl, any of which is optionally substituted by one or more independent G⁴¹ substituents;
R⁶⁹ is halo, —OR⁷⁸, —SH, —NR⁷⁸R⁸⁸, —CO₂R⁷⁸, —C(═O)NR⁷⁸R⁸⁸, —NO₂, —CN, —S(O)ⱼ₈R⁷⁸, —SO₂NR⁷⁸R⁸⁸, C₀₋₁₀alkyl, C₂₋₁₀alkenyl, C₂₋₁₀alkynyl, C₁₋₁₀alkoxyC₁₋₁₀alkyl, C₁₋₁₀alkoxyC₂₋₁₀alkenyl, C₁₋₁₀alkoxyC₂₋₁₀alkynyl, C₁₋₁₀alkylthioC₁₋₁₀alkyl, C₁₋₁₀alkylthioC₂₋₁₀alkenyl, C₁₋₁₀alkylthioC₂₋₁₀alkynyl, cycloC₃₋₈alkyl, cycloC₃₋₈alkenyl, cycloC₃₋₈alkylC₁₋₁₀alkyl, cycloC₃₋₈alkenylC₁₋₁₀alkyl, cycloC₃₋₈alkylC₂₋₁₀alkenyl, cycloC₃₋₈alkenylC₂₋₁₀alkenyl, cycloC₃₋₈alkylC₂₋₁₀alkynyl, cycloC₃₋₈alkenylC₂₋₁₀alkynyl, heterocyclyl-C₀₋₁₀alkyl, heterocyclyl-C₂₋₁₀alkenyl, or heterocyclyl-C₂₋₁₀alkynyl, any of which is optionally substituted with one or more independent halo, cyano, nitro, —OR⁷⁷⁸, —SO₂NR⁷⁷⁸R⁸⁸⁸, or —NR⁷⁷⁸R⁸⁸⁸ substituents;
or R⁶⁹ is aryl-C₀₋₁₀alkyl, aryl-C₂₋₁₀alkenyl, aryl-C₂₋₁₀alkynyl, hetaryl-C₀₋₁₀alkyl, hetaryl-C₂₋₁₀alkenyl, hetaryl-C₂₋₁₀alkynyl, mono(C₁₋₆alkyl)aminoC₁₋₆alkyl, di(C₁₋₆alkyl)aminoC₁₋₆alkyl, mono(aryl)aminoC₁₋₆alkyl, di(aryl)aminoC₁₋₆alkyl, or —N(C₁₋₆alkyl)-C₁₋₆alkyl-aryl, any of which is optionally substituted with one or more independent halo, cyano, nitro, —OR⁷⁷⁸, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —C(═O)NR⁷⁷⁸R⁸⁸⁸, —SO₂NR⁷⁷⁸R⁸⁸⁸, or —NR⁷⁷⁸R⁸⁸⁸ substituents;
or in the case of —NR⁷⁸R⁸⁸, R⁷⁸ and R⁸⁸ are optionally taken together with the nitrogen atom to which they are attached to form a 3-10 membered saturated or unsaturated ring, wherein said ring is optionally substituted with one or more independent halo, cyano, hydroxy, nitro, C₁₋₁₀alkoxy, —SO₂NR⁷⁷⁸R⁸⁸⁸, or —NR⁷⁷⁸R⁸⁸⁸ substituents, and wherein said ring optionally includes one or more heteroatoms other than the nitrogen to which R⁷⁸ and R⁸⁸ are attached;
R⁷⁷, R⁷⁸, R⁸⁷, R⁸⁸, R⁷⁷⁸, and R⁸⁸⁸ are each independently C₀₋₁₀alkyl, C₂₋₁₀alkenyl, C₂₋₁₀alkynyl, C₁₋₁₀alkoxyC₁₋₁₀alkyl, C₁₋₁₀alkoxyC₂₋₁₀alkenyl, C₁₋₁₀alkoxyC₂₋₁₀alkynyl, C₁₋₁₀alkylthioC₁₋₁₀alkyl, C₁₋₁₀alkylthioC₂₋₁₀alkenyl, C₁₋₁₀alkylthioC₂₋₁₀alkynyl, cycloC₃₋₈alkyl, cycloC₃₋₈alkenyl, cycloC₃₋₈alkylC₁₋₁₀alkyl, cycloC₃₋₈alkenylC₁₋₁₀alkyl, cycloC₃₋₈alkylC₂₋₁₀alkenyl, cycloC₃₋₈alkenylC₂₋₁₀alkenyl, cycloC₃₋₈alkylC₂₋₁₀alkynyl, cycloC₃₋₈alkenylC₂₋₁₀alkynyl, heterocyclyl-C₀₋₁₀alkyl, heterocyclyl-C₂₋₁₀alkenyl, heterocyclyl-C₂₋₁₀alkynyl, C₁₋₁₀alkylcarbonyl, C₂₋₁₀alkenylcarbonyl, C₂₋₁₀alkynylcarbonyl, C₁₋₁₀alkoxycarbonyl, C₁₋₁₀alkoxycarbonylC₁₋₁₀alkyl, monoC₁₋₆alkylaminocarbonyl, diC₁₋₆alkylaminocarbonyl, mono(aryl)aminocarbonyl, di(aryl)aminocarbonyl, or C₁₋₁₀alkyl(aryl)aminocarbonyl, any of which is optionally substituted with one or more independent halo, cyano, hydroxy, nitro, C₁₋₁₀alkoxy, —SO₂N(C₀₋₄alkyl)(C₀₋₄alkyl), or —N(C₀₋₄alkyl)(C₀₋₄alkyl) substituents;
or R⁷⁷, R⁷⁸, R⁸⁷, R⁸⁸, R⁷⁷⁸, and R⁸⁸⁸ are each independently aryl-C₀₋₁₀alkyl, aryl-C₂₋₁₀alkenyl, aryl-C₂₋₁₀ alkynyl, hetaryl-C₀₋₁₀alkyl, hetaryl-C₂₋₁₀alkenyl, hetaryl-C₂₋₁₀alkynyl, mono(C₁₋₆alkyl)aminoC₁₋₆alkyl, di(C₁₋₆alkyl)aminoC₁₋₆alkyl, mono(aryl)aminoC₁₋₆alkyl, di(aryl)aminoC₁₋₆alkyl, or —N(C₁₋₆alkyl)-C₁₋₆alkyl-aryl, any of which is optionally substituted with one or more independent halo, cyano, nitro, —O(C₀₋₄alkyl), C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CON(C₀₋₄alkyl)(C₀₋₁₀alkyl), —SO₂N(C₀₋₄alkyl)(C₀₋₄alkyl), or —N(C₀₋₄alkyl)(C₀₋₄alkyl) substituents;
n, m, j1, j1a, j2a, j4, j4a, j5a, j7, and j8 are each independently 0, 1, or 2; and aa and bb are each independently 0 or 1.

In any of the methods of treatment of the invention described herein the patient may be a patient in need of treatment for cancer, including, for example, NSCLC, head and neck squamous cell carcinoma, Ewing's sarcoma, pancreatic, breast or ovarian cancers. In embodiments of any of the methods of treatment of the invention described herein, the cells of the tumors or tumor metastases may be relatively insensitive or refractory to treatment with the anti-cancer agent or treatment as a single agent/treatment.

The present invention also provides a method for the treatment of cancer, comprising administering to a subject in need of such treatment an amount of an anti-cancer agent or treatment that elevates pAkt levels in tumor cells; and an amount of an IGF-1R kinase inhibitor of Formula (I); wherein at least one of the amounts is administered as a sub-therapeutic amount.

The present invention also provides a method for treating tumors or tumor metastases in a patient, comprising administering to said patient simultaneously or sequentially a synergistically effective therapeutic amount of a combination of an anti-cancer agent or treatment that elevates pAkt levels in tumor cells and an IGF-1R kinase inhibitor of Formula (I).

The present invention also provides a method for treating tumors or tumor metastases in a patient refractory to treatment with an anti-cancer agent or treatment that elevates pAkt levels in tumor cells as a single agent, comprising administering to said patient simultaneously or sequentially a therapeutically effective amount of a combination of said anti-cancer agent or treatment and an IGF-1R kinase inhibitor of Formula (I).

The present invention also provides a pharmaceutical composition comprising an anti-cancer agent or treatment that elevates pAkt levels in tumor cells and an IGF-1R kinase inhibitor of Formula (I), in a pharmaceutically acceptable carrier.

The present invention also provides a kit comprising a container, comprising an IGF-1R kinase inhibitor of Formula (I), and an anti-cancer agent or treatment that elevates pAkt levels in tumor cells.

The present invention also provides a method of identifying tumor cells that will respond most favorably to treatment with a combination of an anti-cancer agent or treatment that elevates pAkt levels in tumor cells and an IGF-1R kinase inhibitor, comprising: contacting a sample of tumor cells with said anti-cancer agent or treatment that elevates pAkt levels in tumor cells, determining whether said anti-cancer agent or treatment stimulates phosphorylation of IGF-1R or IR in the tumor cells, by comparing the level of p-IGF-1R or p-IR in tumor cells contacted with said anti-cancer agent or treatment to the level of p-IGF-1R or p-IR in an identical sample of tumor cells either not contacted with said anti-cancer agent or treatment, or contacted with a lower concentration of said anti-cancer agent or treatment, and predicting whether the sample tumor cells will respond favorably to treatment with a combination of an anti-cancer agent or treatment that elevates pAkt levels in tumor cells and an IGF-1R kinase inhibitor, wherein the higher the level of p-IGF-1R or p-IR induced by said anti-cancer agent or treatment in tumor cells, the greater likelihood that the tumor cells will respond favorably to treatment with a combination of an anti-cancer agent or treatment that elevates pAkt levels in tumor cells and an IGF-1R kinase inhibitor.

### BRIEF DESCRIPTION OF THE FIGURES

FIG. 1: Compound D promotes apoptosis induced by doxorubicin in MDA-MB-231 cells: The addition of Compound D (300 nM) to doxorubicin promotes a synergistic induction in apoptosis for MDA-BM-231 cells. Apoptosis measurements were captured 24 hours after dosing, and apoptosis was assayed by measurement of caspase 3/7 activity.

FIG. 2: Compound D inhibits Akt phosphorylation activated by doxorubicin to promote apoptosis: Doxorubicin promotes the phosphorylation of Akt in MDA-MB-231 cells, and this is attenuated by combining doxorubicin with Compound D.

FIG. 3: OSI-906 synergizes with doxorubicin to inhibit cell growth and survival in A673 ES (Ewing's Sarcoma) tumor cells. A. Effect of varying concentrations of doxorubicin, in the presence and absence of 5 μM OSI-906, on the proliferation of A673 tumor cells. Proliferation data were collected 48 hours after dosing and realized using the Cell Titer Glo™ assay (Promega). The dotted line in the plot represents the calculated theoretical expectation if the combination was additive in nature and was determined using the Bliss model for additivity. B. Effect of varying concentrations of OSI-906 (0, 300 nM, and 1 μM) on the induction of apoptosis for A673 tumor cells, in the presence or absence of 333 nM doxorubicin. Apoptosis was determined by measuring caspase 3/7 activity (Caspase GlO™, Promega), and measurements were captured 48 hours after dosing.

FIG. 4: OSI-906 sensitizes SK-ES-1 ES tumor cells to the pro-apoptotic effects of doxorubicin. Effect of varying concentrations of OSI-906 (0, 300 nM, 1 μM, 3 μM, or 5 μM) on the induction of apoptosis for SK-ES-1 tumor cells, in the presence or absence of 1 μM doxorubicin. Apoptosis was determined by measuring caspase 3/7 activity (Caspase GlO™, Promega), and measurements were captured 24 hours after dosing.

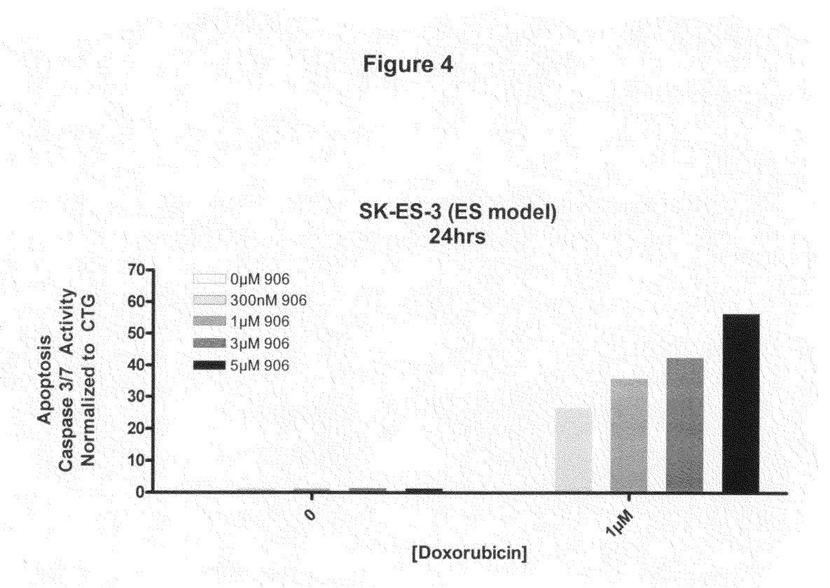

FIG. 5: OSI-906 has the potential to promote greater synergy with doxorubicin in ES tumor cells than neutralizing IGF-1R antibodies. Effect of varying concentrations of OSI-906 (0, 300 nM, 1 μM, and 3 μM) or the IGF-1R neutralizing antibody α-IR3 (10 ug/ml) on the induction of apoptosis for A673 tumor cells, in the presence or absence of 333 nM or 1 uM doxorubicin. Apoptosis was determined by measuring caspase 3/7 activity (Caspase GlO™, Promega), and measurements were captured 24 hours after dosing.

FIG. 6: For ES, doxorubicin treatment results in an increase in pAkt and pErk, which is inhibited by OSI-906. A. Effect of OSI-906 (3 uM), Doxorubicin (500nM or 1 μM), or the combination of OSI-906 and Doxorubicin on the phosphorylation states for Akt, S6, or Erk for A673 tumor cells. B. Phospho-band quantitation for Akt in control, OSI-906, doxorubicin, or combination treated A673 tumor cells. Measurements were collected after 24 hour drug treatment.

FIG. 7: Doxorubicin promotes activation of IGF-1R and IR for A673 ES tumor cells. Effect of OSI-906 (3 μM), Doxorubicin (500 nM), the combination of OSI-906+ doxorubicin, MAB391 (10 ug/ml), and the combination of MAB391+ doxorubicin on the phosphorylation states for IR and IGF-1R. Phosphorylation of IR and IGF-1R was realized using the Proteome Profiler™ RTK capture Array (R & D Systems).

FIG. 8: OSI-906 sensitizes select NSCLC tumor cell lines to taxol. The combination of varying concentrations of OSI-906 (30 nM-3 μM) with 3 nM Taxol on the induction of apoptosis at 24 hours post dosing (Caspase GlO™, Promega) was determined for a panel of 5 NSCLC tumor cell lines (H460, H292, H322, H358, and Calu6). The fold induction in apoptosis greater than the sums achieved by the single agents is noted in the table. An apoptosis gain of >2 was characterized as a significant synergistic interaction, and this was achieved in 3 (H460, H292, and H322) of the 5 tumor cell lines evaluated.

FIG. 9: Taxol promotes an increase in the activation state for IGF-1R for select NSCLC tumor cells, and this correlates with the capacity for OSI-906 to synergize with taxol to inhibit cell survival. The effect of varying concentrations of OSI-906 (0, 0.3 nM, 1 μM, 3 μM, and 5 μM) on apoptosis in the presence or absence of Taxol (3 nM) for H292 and H358 NSCLC tumor cell lines. Apoptosis measurements were determined by measuring caspase 3/7 activity (Caspase GlO™, Promega) and were captured 24 hours after dosing. Also shown is the effect of taxol (100 nM) on IGF-1R phosphorylation for H292 and H358 tumor cells. Phosphorylation was captured 24 hours after dosing with taxol and realized using the Proteome Profiler™ RTK capture Array (R & D Systems).

FIG. 10: The induction in IGF-1R activity by taxol is dose dependent for H292 cells and correlates with a synergistic induction in apoptosis when combined with OSI-906. A. Effect of varying concentrations of taxol on pIGF-1R for H292 tumor cells. pIGF-1R was measured 24 hours after dosing and realized using the proteome profiler RTK capture array (R & D Systems). B. Effect of varying concentrations of taxol, in the presence or absence of 1 μM OSI-906, on the induction of apoptosis for H292 tumor cells. Apoptosis measurements were captured 24 hours after dosing by measuring caspase 3/7 activity (Caspase GlO™, Promega).

FIG. 11: OSI-906 synergizes with taxol to promote apoptosis and block overall cell growth for H460 NSCLC tumor cell lines. A. Effect of varying concentrations of OSI-906, alone or in the presence of 12 nM Taxol, on apoptosis for H460 tumor cells. Apoptosis was measured 24 hours after dosing and determined by caspase 3/7 activity (Caspase GlO™, Promega). B. Effect of varying concentrations of taxol, alone or in the presence of 5 μM OSI-906 on overall cell growth. Overall cell growth was determined by the Cell Titer Glo™ assay (Promega) and measured 72 hours after dosing. The dotted line represents the theoretical expectation for additivity and was calculated using the Bliss model for additivity.

FIG. 12: OSI-906 sensitizes select NSCLC tumor cell lines to cisplatin. The combination of varying concentrations of OSI-906 (30 nM-3 μM) with 50 uM cisplatin on the induction of apoptosis at 24 hours post dosing (Caspase Glo, Promega) was determined for a panel of 5 NSCLC tumor cell lines (H460, H292, H322, H358, and Calu6). The fold induction in apoptosis greater than the sums achieved by the single agents is noted in the table. An apoptosis gain of >2 was characterized as a significant synergistic interaction, and this was achieved in 1 (H292) of the 5 tumor cell lines evaluated.

FIG. 13: OSI-906 synergizes with taxol in SCCHN tumor cell lines (MDA-1186). The effect of varying concentrations of OSI-906 (0, 0.3 nM, 1 uM, 3 μM, and 5 μM) on apoptosis in the presence or absence of Taxol (10 nM) or cisplatin (50 μM) for the MDA-1186 SCCHN tumor cell line. Apoptosis measurements were determined by measuring caspase 3/7 activity (Caspase Glo, Promega) and were captured 24 and 48 hours after dosing.

FIG. 14: Paclitaxel evokes an increase in pIGF-1R in vitro and in vivo. A. H292 NSCLC tumor cells were treated with 30 nM paclitaxel for varying time points, alone or in the presence of 3 μM OSI-906. The phosphorylation of IGF-1R was determined by an RTK capture array, in duplicate (pIGF-1R=phosphorylated IGF-1R). B. Mice bearing H292 xenografts were treated with 24 mg/kg paclitaxel for 24 hours prior to harvesting tumors. Tumors were profiled for pIGF-1R levels using the RTK capture array, in duplicate. Four separate animals were used: Control (Ctrl) A1; Control (Ctrl) A2; Paclitaxel-treated A1; and Paclitaxel-treated A2.

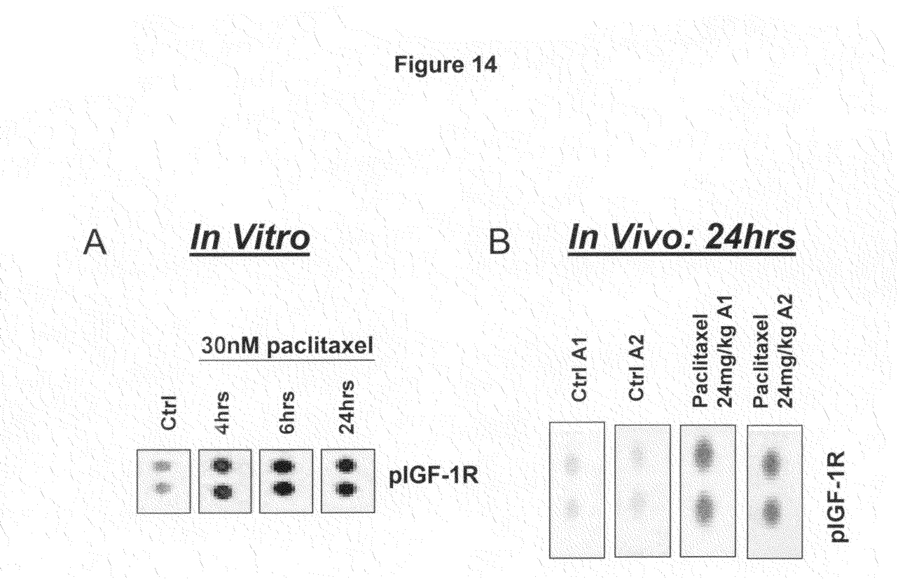

FIG. 15: Paclitaxel evokes a time dependent increase in IGF-1R driven Akt phosphorylation. H292 cells were treated with 30 nM paclitaxel (in the presence and absence of 3 μM OSI-906), and the phosphorylation state for Akt was determined by Western blotting. The phosphorylation of IGF-1R was determined by an RTK capture array, in duplicate (PIGF-1R=phosphorylated IGF-1R).

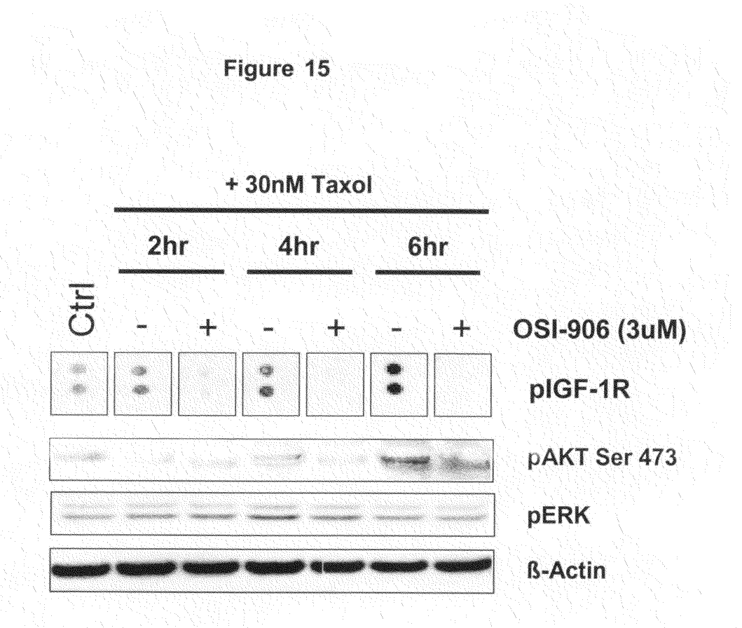

### DETAILED DESCRIPTION OF THE INVENTION

The term “cancer” in an animal refers to the presence of cells possessing characteristics typical of cancer-causing cells, such as uncontrolled proliferation, immortality, metastatic potential, rapid growth and proliferation rate, and certain characteristic morphological features. Often, cancer cells will be in the form of a tumor, but such cells may exist alone within an animal, or may circulate in the blood stream as independent cells, such as leukemic cells.

“Cell growth”, as used herein, for example in the context of “tumor cell growth”, unless otherwise indicated, is used as commonly used in oncology, where the term is principally associated with growth in cell numbers, which occurs by means of cell reproduction (i.e. proliferation) when the rate of the latter is greater than the rate of cell death (e.g. by apoptosis or necrosis), to produce an increase in the size of a population of cells, although a small component of that growth may in certain circumstances be due also to an increase in cell size or cytoplasmic volume of individual cells. An agent that inhibits cell growth can thus do so by either inhibiting proliferation or stimulating cell death, or both, such that the equilibrium between these two opposing processes is altered.

“Tumor growth” or “tumor metastases growth”, as used herein, unless otherwise indicated, is used as commonly used in oncology, where the term is principally associated with an increased mass or volume of the tumor or tumor metastases, primarily as a result of tumor cell growth.

“Abnormal cell growth”, as used herein, unless otherwise indicated, refers to cell growth that is independent of normal regulatory mechanisms (e.g., loss of contact inhibition). This includes the abnormal growth of: (1) tumor cells (tumors) that proliferate by expressing a mutated tyrosine kinase or over-expression of a receptor tyrosine kinase; (2) benign and malignant cells of other proliferative diseases in which aberrant tyrosine kinase activation occurs; (4) any tumors that proliferate by receptor tyrosine kinases; (5) any tumors that proliferate by aberrant serine/threonine kinase activation; and (6) benign and malignant cells of other proliferative diseases in which aberrant serine/threonine kinase activation occurs.

The term “treating” as used herein, unless otherwise indicated, means reversing, alleviating, inhibiting the progress of, or preventing, either partially or completely, the growth of tumors, tumor metastases, or other cancer-causing or neoplastic cells in a patient. The term “treatment” as used herein, unless otherwise indicated, refers to the act of treating.

The phrase “a method of treating” or its equivalent, when applied to, for example, cancer refers to a procedure or course of action that is designed to reduce or eliminate the number of cancer cells in an animal, or to alleviate the symptoms of a cancer. “A method of treating” cancer or another proliferative disorder does not necessarily mean that the cancer cells or other disorder will, in fact, be eliminated, that the number of cells or disorder will, in fact, be reduced, or that the symptoms of a cancer or other disorder will, in fact, be alleviated. Often, a method of treating cancer will be performed even with a low likelihood of success, but which, given the medical history and estimated survival expectancy of an animal, is nevertheless deemed an overall beneficial course of action.

The term “therapeutically effective agent” means a composition that will elicit the biological or medical response of a tissue, system, animal or human that is being sought by the researcher, veterinarian, medical doctor or other clinician.

The term “therapeutically effective amount” or “effective amount” means the amount of the subject compound or combination that will elicit the biological or medical response of a tissue, system, animal or human that is being sought by the researcher, veterinarian, medical doctor or other clinician.

The term “method for manufacturing a medicament” or “use of for manufacturing a medicament” relates to the manufacturing of a medicament for use in the indication as specified herein, and in particular for use in tumors, tumor metastases, or cancer in general. The term relates to the so-called “Swiss-type” claim format in the indication specified.

The data presented in the Examples herein below demonstrate that IGF-1R kinase inhibitors of Formula (I) are agents that potentiate the pro-apoptotic affects of anti-cancer agents or treatments that elevate pAkt levels in tumor cells, and whose effectiveness is thus limited by this property. Thus the anti-tumor effects of a combination of an anti-cancer agent or treatment that elevates pAkt levels in tumor cells and an IGF-1R kinase inhibitor of Formula (I) are superior to the anti-tumor effects of either anti-cancer agent by itself, and co-administration of these agents can be effective for treatment of patients with advanced cancers such as NSCL, pancreatic, head and neck, colon, ovarian, and breast cancers, and Ewing's sarcoma. This combination was consistently found to produce a synergistic effect in inhibiting the growth of tumor cells or promoting an induction in apoptosis of tumor cells, presumably due to the ability of these new IGF-1R kinase inhibitors to inhibit Akt activation.

Accordingly, the present invention provides a method for treating tumors or tumor metastases in a patient, comprising administering to said patient simultaneously or sequentially a therapeutically effective amount of a combination of an anti-cancer agent or treatment that elevates pAkt levels in tumor cells and an IGF-1R kinase inhibitor of Formula (I). In one embodiment the patient is a human that is being treated for cancer. In different embodiments, the anti-cancer agent or treatment and IGF-1R kinase inhibitor of Formula (I) are co-administered to the patient in the same formulation; are co-administered to the patient in different formulations; are co-administered to the patient by the same route; or are co-administered to the patient by different routes. In another embodiment one or more other anti-cancer agents can additionally be administered to said patient.

In a preferred embodiment of the preceding methods for treating tumors or tumor metastases in a patient, the anti-cancer agent or treatment that elevates pAkt levels in tumor cells and the IGF-1R kinase inhibitor of Formula (I) are administered sequentially, and the anti-cancer agent or treatment that elevates pAkt levels in tumor cells is administered prior to the IGF-1R kinase inhibitor. In one example of this embodiment, the anti-cancer agent or treatment that elevates pAkt levels in tumor cells is administered at least two hours prior to the IGF-1R kinase inhibitor. Alternatively, the anti-cancer agent or treatment that elevates pAkt levels in tumor cells is administered at least four, at least six, at least twelve or at least twenty-four hours prior to the IGF-1R kinase inhibitor.

In any of the methods, compositions or kits of the invention described herein, an anti-cancer agent or treatment that elevates pAkt levels in tumor cells can be any anti-cancer agent or treatment presently known or yet to be characterized that elevates pAkt levels in tumor cells. In one embodiment, the anti-cancer agent or treatment that elevates pAkt levels is a chemotherapeutic agent. Examples of such chemotherapeutic agents that elevate pAkt levels include anthracyclins, such as doxorubicin or daunorubicin; tamoxifen; DNA-damaging agents, such as cisplatin or carboplatin; topoisomerase inhibitors, such as camptothecin or etoposide; and microtubule-directed agents, such as vincristine, colchicines, vinblastine, decetaxel, and paclitaxel. In another embodiment, the anti-cancer agent or treatment that elevates pAkt levels is a form of ionizing radiation. In an other embodiment, the anti-cancer agent or treatment that elevates pAkt levels is a gene-targetted anti-cancer agent. Examples of such gene-targeted anti-cancer agents that elevate pAkt levels include rapamycin; rapalogs (i.e. rapamycin analogs), such as CCI-779 or RAD001; trastuzumab; and the pan-Akt inhibitor A443654.

In any of the methods, compositions or kits of the invention described herein, the IGF-1R kinase inhibitor of Formula (I) can be any IGF-1R kinase inhibitor compound encompassed by Formula (I) that inhibits IGF-1R kinase upon administration to a patient. Examples of such inhibitors have been published in US Published Patent Application US 2006/0235031, which is incorporated herein in its entirety, and include Compound D (OSI-906) as used in the experiments described herein.

An IGF-1R kinase inhibitor of Formula (I) is represented by the formula:

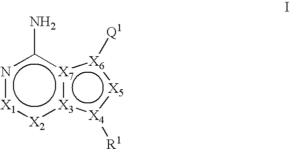

or a pharmaceutically acceptable salt thereof, wherein:
X₁, and X₂ are each independently N or C-(E¹)ₐₐ;
X₅ is N, C-(E¹)ₐₐ, or N-(E¹)ₐₐ;
X₃, X₄, X₆, and X₇ are each independently N or C;
   wherein at least one of X₃, X₄, X₅, X₆, and X₇ is independently N or N-(E¹)ₐₐ;
Q¹ is

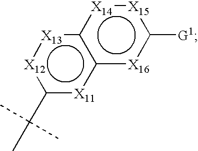

X₁₁, X₁₂, X₁₃, X₁₄, X₁₅, and X₁₆ are each independently N, C-(E¹¹)_{bb}, or N⁺—O⁻;
wherein at least one of X₁₁, X₁₂, X₁₃, X₁₄, X₁₅, and X₁₆ is N or N⁺—O⁻;
R¹ is absent, C₀₋₁₀alkyl, cycloC₃₋₁₀alkyl, bicycloC₅₋₁₀alkyl, aryl, heteroaryl, aralkyl, heteroaralkyl, heterocyclyl, heterobicycloC₅₋₁₀alkyl, spiroalkyl, or heterospiroalkyl, any of which is optionally substituted by one or more independent G¹¹ substituents;
E¹, E¹¹, G¹, and G⁴¹ are each independently halo, —CF₃, —OCF₃, —OR², —NR²R³(R^{2a})ⱼ₁, —C(═O)R², —CO₂R², —CONR²R³, —NO₂, —CN, —S(O)ⱼ₁R², —SO₂NR²R³, —NR²C(═O)R³, —NR²C(═O)OR³, —NR²C(═O)NR³R^{2a}, —NR²S(O)ⱼ₁R³, —C(═S)OR², —CO)SR², —NR²C(═NR³)NR^{2a}R^{3a}, —NR²C(═NR³)OR^{2a}, —NR²C(═NR³)SR^{2a}, —OC(═O)OR², —OC(═O)NR²R³, —OC(═O)SR², —SC(═O)OR², —SC(═O)NR²R³, C₀₋₁₀alkyl, C₂₋₁₀alkenyl, C₂₋₁₀alkynyl, C₁₋₁₀alkoxyC₁₋₁₀alkyl, C₁₋₁₀alkoxyC₂₋₁₀alkenyl, C₁₋₁₀alkoxyC₂₋₁₀alkynyl, C₁₋₁₀alkylthioC₁₋₁₀alkyl, C₁₋₁₀alkylthioC₂₋₁₀alkenyl, C₁₋₁₀alkylthioC₂₋₁₀alkynyl, cycloC₃₋₈alkyl, cycloC₃₋₈alkenyl, cycloC₃₋₈alkylC₁₋₁₀alkyl, cycloC₃₋₈alkenylC₁₋₁₀alkyl, cycloC₃₋₈alkylC₂₋₁₀alkenyl, cycloC₃₋₈alkenylC₂₋₁₀ alkenyl, cycloC₃₋₈alkylC₂₋₁₀alkynyl, cycloC₃₋₈alkenylC₂₋₁₀alkynyl, heterocyclyl-C₀₋₁₀alkyl, heterocyclyl-C₂₋₁₀alkenyl, or heterocyclyl-C₂₋₁₀alkynyl, any of which is optionally substituted with one or more independent halo, oxo, —CF₃, —OCF₃, —OR²²², —NR²²²R³³³(R^{222a})ⱼ₁ₐ, —C(═O)R²²², —CO₂R²²²—C(═O)NR²²²R³³³, —NO₂, —CN, —S(═O)ⱼ₁ₐR²²², —SO₂NR²²²R³³³, —NR²²²C(═O)R³³³, —NR²²²C(═O)OR³³³, —NR²²²C(═O)NR³³³R^{222a}, —NR²²²S(O)ⱼ₁ₐR³³³, —C(═S)OR²²², —C(═O)SR²²², —NR²²²C(═NR³³³)NR^{222a}R^{333a}, —NR²²²C(═NR³³³)OR^{222a}, —NR²²²C(═NR³³³)SR^{222a}, —OC(═O)OR²²², —OC(═O)NR²²²R³³³, —OC(═O)SR²²², —SC(═O)OR²²², or —SC(═O)NR²²²R³³³ substituents;
or E¹, E¹¹, or G¹ optionally is —(W¹)ₙ—(Y¹)ₘ—R⁴;
or E¹, E¹¹, G¹, or G⁴¹ optionally independently is aryl-C₀₋₁₀alkyl, aryl-C₂₋₁₀alkenyl, aryl-C₂₋₁₀alkynyl, hetaryl-C₀₋₁₀alkyl, hetaryl-C₂₋₁₀alkenyl, or hetaryl-C₂₋₁₀alkynyl, any of which is optionally substituted with one or more independent halo, —CF₃, —OCF₃ OR²²², —NR²²²R³³³(R^{222a})ⱼ₂ₐ, —C(O)R²²², —CO₂R²²², —C(═O)NR²²²R³³³, —NO₂, —CN, —S(O)ⱼ₂ₐR²²², —SO₂NR²²²R³³³, —NR²²²C(═O)R³³³, —NR²²²C(═O)OR³³³, —NR²²²C(═O)NR³³³R^{222a}, —NR²²²S(O)ⱼ₂ₐR³³³, —C(═S)OR²²², —C(═O)SR²²², —NR²²²C(═NR³³³)NR^{222a}R^{333a}, —NR²²²C(═NR³³³)OR^{222a}, —NR²²²C(═NR³³³)SR^{222a}, —OC(═O)OR²²², —OC(═O)NR²²²R³³³, —OC(═O)SR²²², —SC(═O)OR²²², or —SC(═O)NR²²²R³³³ substituents;
G¹¹ is halo, oxo, —CF₃, —OCF₃, —OR²¹, —NR²¹R³¹ (R^{2a1})ⱼ₄, —C(O)R²¹, —CO₂R²¹, —C(═O)NR²¹R³¹, —NO₂, —CN, —S(O)ⱼ₄R²¹, —SO₂NR²¹R³¹, NR²¹(C═O)R³¹, NR²¹C(═O)OR³¹, NR²¹C(═O)NR³¹R^{2a1}, NR²¹S(O)ⱼ₄R³¹, —C(═S)OR²¹, —C(═O)SR²¹, —NR²¹C(═NR³¹)NR^{2a1}R^{3a1}, —NR²¹C(═NR³¹)OR^{2a1}, —NR²¹C(═NR³¹)SR^{2a1}, —OC(═O)OR²¹, —OC(═O)NR²¹R³¹, —OC(═O)SR²¹, —SC(═O)OR²¹, —SC(═O)NR²¹R³¹, —P(O)OR²¹OR³¹, C₁₋₁₀alkylidene, C₀₋₁₀alkyl, C₂₋₁₀alkenyl, C₂₋₁₀alkynyl, C₁₋₁₀alkoxyC₁₋₁₀alkyl, C₁₋₁₀alkoxyC₂₋₁₀alkenyl, C₁₋₁₀alkoxyC₂₋₁₀alkynyl, C₁₋₁₀alkylthioC₁₋₁₀alkyl, C₁₋₁₀alkylthioC₂₋₁₀alkenyl, C₁₋₁₀alkylthioC₂₋₁₀alkynyl, cycloC₃₋₈alkyl, cycloC₃₋₈alkenyl, cycloC₃₋₈alkylC₁₋₁₀alkyl, cycloC₃₋₈alkenylC₁₋₁₀alkyl, cycloC₃₋₈alkylC₂₋₁₀alkenyl, cycloC₃₋₈alkenylC₂₋₁₀alkenyl, cycloC₃₋₈alkylC₂₋₁₀alkynyl, cycloC₃₋₈alkenylC₂₋₁₀alkynyl, heterocyclyl-C₀₋₁₀alkyl, heterocyclyl-C₂₋₁₀alkenyl, or heterocyclyl-C₂₋₁₀alkynyl, any of which is optionally substituted with one or more independent halo, oxo, —CF₃, —OCF₃, —OR²²²¹, —NR²²²¹R³³³¹(R^{222a1})ⱼ₄ₐ, —C(O)R²²²¹, —CO₂R²²²¹, —C(═O)NR²²²¹R³³³¹, —NO₂, —CN, —S(O)ⱼ₄ₐR²²²¹, —SO₂NR²²²¹R³³³¹, —NR²²²¹C(═O)R³³³¹, —NR²²²¹C(═O)OR³³³¹, —NR²²²¹ C(═O)NR³³³¹R^{222a1}, —NR²²²¹S(O)ⱼ₄ₐR³³³¹, —C(═S)OR²²²¹, —C(═O)SR²²²¹, —NR²²²¹C(═NR³³³¹)NR^{222a1}R^{333a1}, —NR²²²¹C(═NR³³³¹)OR^{222a1}, —NR²²²¹C(═NR³³³¹)SR^{222a1}, —OC(═O)OR²²²¹, —OC(═O)NR²²²¹R³³³¹, —OC(═O)SR²²²¹, —SC(═O)OR²²²¹, —P(O)OR²²²¹OR³³³¹, or —SC(═O)NR²²²¹R³³³¹ substituents;
or G¹¹ is aryl-C₀₋₁₀alkyl, aryl-C₂₋₁₀alkenyl, aryl-C₂₋₁₀alkynyl, hetaryl-C₀₋₁₀alkyl, hetaryl-C₂₋₁₀alkenyl, or hetaryl-C₂₋₁₀alkynyl, any of which is optionally substituted with one or more independent halo, —CF₃, —OCF₃, —OR²²²¹, NR²²²¹R³³³¹(R^{222a1})ⱼ₅ₐ, —C(O)R²²²¹, —CO₂R²²²¹, —C(═O)NR²²²¹R³³³¹, —NO₂, —CN, —S(O)ⱼ₅ₐR²²²¹, —SO₂NR²²²¹R³³³¹, —NR²²²¹C(═O)R³³³¹, —NR²²²¹C(═O)OR³³³¹, —NR²²²¹C(═O)NR³³³¹R^{222a1}, —NR²²²¹S(O)ⱼ₅ₐR³³³¹, —C(═S)OR²²²¹, —C(═O)SR²²²¹, —NR²²²¹C(═NR³³³¹)NR^{22a1}R^{333a1}, —NR²²²¹C(═NR³³³¹)OR^{222a1}, —NR²²²¹C(═NR³³³¹)SR^{222a1}, —OC(═O)OR²²²¹, —OC(═O)NR²²²¹R³³³¹, —OC(═O)SR²²²¹, —SC(═O)OR²²²¹, —P(O)OR²²²¹OR³³³¹, or —SC(═O)NR²²²¹R³³³¹ substituents;
or G¹¹ is C, taken together with the carbon to which it is attached forms a C═C double bond which is substituted with R⁵ and G¹¹¹;
R², R^{2a}, R³, R^{3a}, R²²², R^{222a}, R³³³, R^{333a}, R²¹, R^{2a1}, R³¹, R^{3a1}, R²²²¹, R^{222a1}, R³³³¹, and R^{333a1} are each independently C₀₋₁₀alkyl, C₂₋₁₀alkenyl, C₂₋₁₀alkynyl, C₁₋₁₀alkoxyC₁₋₁₀alkyl, C₁₋₁₀alkoxyC₂₋₁₀alkenyl, C₁₋₁₀alkoxyC₂₋₁₀alkynyl, C₁₋₁₀alkylthioC₁₋₁₀alkyl, C₁₋₁₀alkylthioC₂₋₁₀alkenyl, C₁₋₁₀alkylthioC₂₋₁₀alkynyl, cycloC₃₋₈alkyl, cycloC₃₋₈alkenyl, cycloC₃₋₈alkylC₁₋₁₀alkyl, cycloC₃₋₈alkenylC₁₋₁₀alkyl, cycloC₃₋₈alkylC₂₋₁₀alkenyl, cycloC₃₋₈alkenylC₂₋₁₀ alkenyl, cycloC₃₋₈alkylC₂₋₁₀alkynyl, cycloC₃₋₈alkenylC₂₋₁₀alkynyl, heterocyclyl-C₀₋₁₀alkyl, heterocyclyl-C₂₋₁₀alkenyl, heterocyclyl-C₂₋₁₀alkynyl, aryl-C₀₋₁₀alkyl, aryl-C₂₋₁₀alkenyl, or aryl-C₂₋₁₀alkynyl, hetaryl-C₀₋₁₀alkyl, hetaryl-C₂₋₁₀alkenyl, or hetaryl-C₂₋₁₀ alkynyl, any of which is optionally substituted by one or more independent G¹¹¹ substituents;
or in the case of —NR²R³(R^{2a})ⱼ₁ or —NR²²²R³³³(R^{222a})ⱼ₁ₐ or —NR²²²¹R³³³(R^{222a})ⱼ₂ₐ or —NR²¹R³¹(R^{2a1})ⱼ₄ or —NR²²²¹R³³³¹(R^{222a1})ⱼ₄ₐ or —NR²²²¹R³³³¹(R^{222a1})ⱼ₅ₐ, then R² and R³, or R²²² and R³³³, or R²²²¹ and R³³³¹, respectfully, are optionally taken together with the nitrogen atom to which they are attached to form a 3-10 membered saturated or unsaturated ring, wherein said ring is optionally substituted by one or more independent G¹¹¹¹ substituents and wherein said ring optionally includes one or more heteroatoms other than the nitrogen to which R² and R³, or R²²² and R³³³, or R²²¹ and R³³³¹ are attached;
W¹ and Y¹ are each independently —O—, —NR⁷—, —S(O)ⱼ₇—, —CR⁵R⁶—, —N(C(O)OR⁷)—, —N(C(O)R⁷)—, —N(SO₂R⁷)—, —CH₂O—, —CH₂S—, —CH₂N(R⁷)—, —CH(NR⁷)—, —CH₂N(C(O)R⁷)—, —CH₂N(C(O)OR⁷)—, —CH₂N(SO₂R⁷)—, —CH(NHR⁷)—, —CH(NHC(O)R⁷)—, —CH(NHSO₂R⁷)—, —CH(NHC(O)OR⁷)—, —CH(OC(O)R⁷)—, —CH(OC(O)NHR⁷)—, —CH═CH—, —C≡C—, —C(═NOR⁷)—, —C(O)—, —CH(OR⁷)—, —C(O)N(R⁷)—, —N(R⁷)C(O)—, —N(R⁷)S(O)—, —N(R⁷)S(O)₂—, —OC(O)N(R⁷)—, —N(R⁷)C(O)N(R⁸)—, —NR⁷C(O)O—, —S(O)N(R⁷)—, —S(O)₂N(R⁷)—, —N(C(O)R⁷)S(O)—, —N(C(O)R⁷)S(O)₂—, —N(R⁷)S(O)N(R⁸)—, —N(R⁷)S(O)₂N(R⁸)—, —C(O)N(R⁷)C(O)—, —S(O)N(R⁷)C(O)—, —S(O)₂N(R⁷)C(O)—, —OS(O)N(R⁷)—, O—S(O)₂N(R⁷)—, —N(R⁷)S(O)O—, —N(R⁷) S(O)₂O—, —N(R⁷)S(O)C(O)—, —N(R⁷)S(O)₂C(O)—, —SON(C(O)R⁷)—, —SO₂N(C(O)R⁷)—, —N(R⁷)SON(R⁸)—, —N(R⁷)SO₂N(R⁸)—, —C(O)O—, —N(R⁷)P(OR⁸)O—, —N(R⁷)P(OR⁸)—, —N(R⁷)P(O)(OR⁸)O—, —N(R⁷)P(O)(OR⁸)—, —N(C(O)R⁷)P(OR⁸)O—, —N(C(O)R⁷)P(OR⁸)—, —N(C(O)R⁷)P(O)(OR⁸)O—, —N(C(O)R⁷)P(OR⁸)—, —CH(R⁷)S(O)—, —CH(R⁷)S(O)₂—, —CH(R⁷)N(C(O)OR⁸)—, —CH(R⁷)N(C(O)R⁸)—, —CH(R⁷)N(SO₂R⁸)—, —CH(R⁷)O—, —CH(R⁷)S—, —CH(R⁷)N(R⁸)—, —CH(R⁷)N(C(O)R⁸)—, —CH(R⁷)N(C(O)OR⁸)—, —CH(R⁷)N(SO₂R⁸)—, —CH(R⁷)C(═NOR⁸)—, —CH(R⁷)C(O)—, —CH(R⁷)CH(OR⁸)—, —CH(R⁷)C(O)N(R⁸)—, —CH(R⁷)N(R⁸)C(O)—, —CH(R⁷)N(R⁸)S(O)—, —CH(R⁷)N(R⁸)S(O)₂—, —CH(R⁷)OC(O)N(R⁸)—, —CH(R⁷)N(R⁸)C(O)N(R^{7a})—, —CH(R⁷)NR⁸C(O)O—, —CH(R⁷)S(O)N(R⁸)—, —CH(R⁷)S(O)₂N(R⁸)—, —CH(R⁷)N(C(O)R⁸)S(O)—, —CH(R⁷)N(C(O)R⁸)S(O)—, —CH(R⁷)N(R⁸)S(O)N(R^{7a})—, —CH(R⁷)N(R⁸)S(O)₂N(R^{7a})—, —CH(R⁷)C(O)N(R⁸)C(O)—, —CH(R⁷)S(O)N(R⁸)C(O)—, —CH(R⁷)S(O)₂N(R⁸)C(O)—, —CH(R⁷)OS(O)N(R⁸)—, —CH(R⁷)OS(O)₂N(R⁸)—, —CH(R⁷)N(R⁸)S(O)O—, —CH(R⁷)N(R⁸)S(O)₂O—, —CH⁷)N(R⁸)S(O)C(O)—, —CH(R⁷)N(R⁸)S(O)₂C(O)—, —CH(R⁷)SON(C(O)R⁸)—, —CH(R⁷)SO₂N(C(O)R⁸)—, —CH(R⁷)N(R⁸)SON(R^{7a})—, —CH(R⁷)N(R⁸)SO₂N(R^{7a})—, —CH(R⁷)C(O)O—, —CH(R⁷)N(R⁸)P(OR^{7a})—, —CH(R⁷)N(R⁸)P(OR^{7a})—, —CH(R⁷)N(R⁸)P(O)(OR^{7a})O—, —CH(R⁷)N(R⁸)P(O)(OR^{7a})—, —CH(R⁷)N(C(O)R⁸)P(OR^{7a})O—, —CH(R⁷)N(C(O)R⁸)P(OR^{7a})—, —CH(R⁷)N(C(O)R⁸)P(O)(OR^{7a})O—, or —CH(R⁷)N(C(O)R⁸)P(OR^{7a})—;
R⁵, R⁶, G¹¹¹, and G¹¹¹¹ are each independently C₀₋₁₀alkyl, C₂₋₁₀alkenyl, C₂₋₁₀alkynyl, C₁₋₁₀alkoxyC₁₋₁₀alkyl, C₁₋₁₀alkoxyC₂₋₁₀alkenyl, C₁₋₁₀alkoxyC₂₋₁₀alkynyl, C₁₋₁₀alkylthioC₁₋₁₀alkyl, C₁₋₁₀alkylthioC₂₋₁₀alkenyl, C₁₋₁₀alkylthioC₂₋₁₀alkynyl, cycloC₃₋₈alkyl, cycloC₃₋₈alkenyl, cycloC₃₋₈alkylC₁₋₁₀alkyl, cycloC₃₋₈alkenylC₁₋₁₀alkyl, cycloC₃₋₈alkylC₂₋₁₀alkenyl, cycloC₃₋₈alkenylC₂₋₁₀alkenyl, cycloC₃₋₈alkylC₂₋₁₀alkynyl, cycloC₃₋₈alkenylC₂₋₁₀alkynyl, heterocyclyl-C₀₋₁₀alkyl, heterocyclyl-C₂₋₁₀alkenyl, heterocyclyl-C₂₋₁₀alkynyl, aryl-C₀₋₁₀alkyl, aryl-C₂₋₁₀alkenyl, aryl-C₂₋₁₀alkynyl, hetaryl-C₀₋₁₀alkyl, hetaryl-C₂₋₁₀alkenyl, or hetaryl-C₂₋₁₀ alkynyl, any of which is optionally substituted with one or more independent halo, —CF₃, —OCF₃, —OR⁷⁷, —NR⁷⁷R⁸⁷, —C(O)R⁷⁷, —CO₂R⁷⁷, —CONR⁷⁷R⁸⁷, —NO₂, —CN, —S(O)ⱼ₅ₐR⁷⁷, —SO₂NR⁷⁷R⁸⁷, —NR⁷⁷C(═O)R⁸⁷, —NR⁷⁷C(═O)OR⁸⁷, —NR⁷⁷C(═O)NR⁷⁸R⁸⁷, —NR⁷⁷S(O)ⱼ₅ₐR⁷—C(═S)OR⁷⁷, —C(═O)SR⁷⁷, —NR⁷⁷C(═NR⁸⁷)NR⁷⁸R⁸⁸, —NR⁷⁷C(═NR⁸⁷)OR⁷⁸, —NR⁷⁷C(═NR⁸⁷)SR⁷⁸, —OC(═O)OR⁷⁷, —OC(═O)NR⁷⁷R⁸⁷, —OC(═O)SR⁷⁷, —SC(═O)OR⁷⁷, —P(O)OR⁷⁷OR⁸⁷, or —SC(═O)NR⁷⁷R⁸⁷ substituents;
or R⁵ with R⁶ are optionally taken together with the carbon atom to which they are attached to form a 3-10 membered saturated or unsaturated ring, wherein said ring is optionally substituted with one or more independent R⁶⁹ substituents and wherein said ring optionally includes one or more heteroatoms;
R⁷, R^{7a}, and R⁸ are each independently acyl, C₀₋₁₀alkyl, C₂₋₁₀alkenyl, aryl, heteroaryl, heterocyclyl or cycloC₃₋₁₀alkyl, any of which is optionally substituted by one or more independent G¹¹¹ substituents;
R⁴ is C₀₋₁₀alkyl, C₂₋₁₀alkenyl, C₂₋₁₀alkynyl, aryl, heteroaryl, cycloC₃₋₁₀alkyl, heterocyclyl, cycloC₃₋₈alkenyl, or heterocycloalkenyl, any of which is optionally substituted by one or more independent G⁴¹ substituents;
R⁶⁹ is halo, —OR⁷⁸, —SH, —NR⁷⁸R⁸⁸, —CO₂R⁷⁸, —C(═O)NR⁷⁸R⁸⁸—NO₂, —CN, —S(O)ⱼ₈R⁷⁸, —SO₂NR⁷⁸R⁸⁸, C₀₋₁₀alkyl, C₂₋₁₀alkenyl, C₂₋₁₀alkynyl, C₁₋₁₀alkoxyC₁₋₁₀alkyl, C₁₋₁₀alkoxyC₂₋₁₀alkenyl, C₁₋₁₀alkoxyC₂₋₁₀alkynyl, C₁₋₁₀alkylthioC₁₋₁₀alkyl, C₁₋₁₀alkylthioC₂₋₁₀ alkenyl, C₁₋₁₀alkylthioC₂₋₁₀alkynyl, cycloC₃₋₈alkyl, cycloC₃₋₈alkenyl, cycloC₃₋₈alkylC₁₋₁₀alkyl, cycloC₃₋₈alkenylC₁₋₁₀alkyl, cycloC₃₋₈alkylC₂₋₁₀alkenyl, cycloC₃₋₈alkenylC₂₋₁₀ alkenyl, cycloC₃₋₈alkylC₂₋₁₀alkynyl, cycloC₃₋₈alkenylC₂₋₁₀alkynyl, heterocyclyl-C₀₋₁₀alkyl, heterocyclyl-C₂₋₁₀alkenyl, or heterocyclyl-C₂₋₁₀alkynyl, any of which is optionally substituted with one or more independent halo, cyano, nitro, —OR⁷⁷⁸, —SO₂NR⁷⁷⁸R⁸⁸⁸, or —NR⁷⁷⁸R⁸⁸⁸ substituents;
or R⁶⁹ is aryl-C₀₋₁₀alkyl, aryl-C₂₋₁₀alkenyl, aryl-C₂₋₁₀alkynyl, hetaryl-C₀₋₁₀alkyl, hetaryl-C₂₋₁₀alkenyl, hetaryl-C₂₋₁₀alkynyl, mono(C₁₋₆alkyl)aminoC₁₋₆alkyl, di(C₁₋₆alkyl)aminoC₁₋₆alkyl, mono(aryl)aminoC₁₋₆alkyl, di(aryl)aminoC₁₋₆alkyl, or —N(C₁₋₆alkyl)-C₁₋₆alkyl-aryl, any of which is optionally substituted with one or more independent halo, cyano, nitro, —OR⁷⁷⁸, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —C(═O)NR⁷⁷⁸R⁸⁸⁸, —SO₂NR⁷⁷⁸R⁸⁸⁸, or —NR⁷⁷⁸R⁸⁸⁸ substituents;
or in the case of —NR⁷⁸R⁸⁸, R⁷⁸ and R⁸⁸ are optionally taken together with the nitrogen atom to which they are attached to form a 3-10 membered saturated or unsaturated ring, wherein said ring is optionally substituted with one or more independent halo, cyano, hydroxy, nitro, C₁₋₁₀alkoxy, —SO₂NR⁷⁷⁸R⁸⁸⁸ or —NR⁷⁷⁸R⁸⁸⁸ substituents, and wherein said ring optionally includes one or more heteroatoms other than the nitrogen to which R⁷⁸ and R⁸⁸ are attached;
R⁷⁷R⁷⁸R⁸⁷R⁸⁸R⁷⁷⁸ and R⁸⁸⁸ are each independently C₀₋₁₀alkyl, C₂₋₁₀alkenyl, C₂₋₁₀alkynyl, C₁₋₁₀alkoxyC₁₋₁₀alkyl, C₁₋₁₀alkoxyC₂₋₁₀alkenyl, C₁₋₁₀alkoxyC₂₋₁₀alkynyl, C₁₋₁₀alkylthioC₁₋₁₀alkyl, C₁₋₁₀alkylthioC₂₋₁₀alkenyl, C₁₋₁₀alkylthioC₂₋₁₀alkynyl, cycloC₃₋₈alkyl, cycloC₃₋₈alkenyl, cycloC₃₋₈alkylC₁₋₁₀alkyl, cycloC₃₋₈alkenylC₁₋₁₀alkyl, cycloC₃₋₈alkylC₂₋₁₀alkenyl, cycloC₃₋₈alkenylC₂₋₁₀alkenyl, cycloC₃₋₈alkylC₂₋₁₀alkynyl, cycloC₃₋₈alkenylC₂₋₁₀alkynyl, heterocyclyl-C₀₋₁₀alkyl, heterocyclyl-C₂₋₁₀alkenyl, heterocyclyl-C₂₋₁₀alkynyl, C₁₋₁₀alkylcarbonyl, C₂₋₁₀alkenylcarbonyl, C₂₋₁₀alkynylcarbonyl, C₁₋₁₀alkoxycarbonyl, C₁₋₁₀alkoxycarbonylC₁₋₁₀alkyl, monoC₁₋₆alkylaminocarbonyl, diC₁₋₆alkylaminocarbonyl, mono(aryl)aminocarbonyl, di(aryl)aminocarbonyl, or C₁₋₁₀alkyl(aryl)aminocarbonyl, any of which is optionally substituted with one or more independent halo, cyano, hydroxy, nitro, C₁₋₁₀alkoxy, —SO₂N(C₀₋₄alkyl)(C₀₋₄alkyl), or —N(C₀₋₄alkyl)(C₀₋₄alkyl) substituents;
or R⁷⁷, R⁷⁸, R⁸⁷, R⁸⁸, R⁷⁷⁸, and R⁸⁸⁸ are each independently aryl-C₀₋₁₀alkyl, aryl-C₂₋₁₀alkenyl, aryl-C₂₋₁₀alkynyl, hetaryl-C₀₋₁₀alkyl, hetaryl-C₂₋₁₀alkenyl, hetaryl-C₂₋₁₀alkynyl, mono(C₁₋₆alkyl)aminoC₁₋₆alkyl, di(C₁₋₆alkyl)aminoC₁₋₆alkyl, mono(aryl)aminoC₁₋₆alkyl, di(aryl)aminoC₁₋₆alkyl, or —N(C₁₋₆alkyl)-C₁₋₆alkyl-aryl, any of which is optionally substituted with one or more independent halo, cyano, nitro, —O(C₀₋₄alkyl), C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, C₁₋₄alkoxycarbonyl, —CON(C₀₋₄alkyl)(C₀₋₁₀alkyl), —SO₂N(C₀₋₄alkyl)(C₀₋₄alkyl), or —N(C₀₋₄alkyl)(C₀₋₄alkyl) substituents;
n, m, j1, j1a, j2a, j4, j4a, j5a, j7, and j8 are each independently 0, 1, or 2; and aa and bb are each independently 0 or 1.

IGF-1R kinase inhibitor compounds of Formula (I), such as Compound D (OSI-906), have a number of important advantages over other compounds that inhibit the IGF-1R signaling pathway. These include: (a) They are low molecular weight inhibitors and therefore, should be easier to dose in combination with other inhibitors (e.g. antibody inhibitors) because of the ease of scheduling. Antibody IGF 1-R inhibitors, for example, have effects that persist for extended periods of time, which severely limits scheduling regimens with other anti-cancer agents. (b) Many, such as Compound D (OSI-906), are more selective by at least 10-fold toward IGF-1R than IR (insulin receptor kinase) than other small molecule IGF-R kinase inhibitors, lessening the potential for toxic side effects that occur via IR, that could for example adversely affect glucose metabolism and transport. (c) Although more selective toward IGF-1R than IR, these compounds (e.g. Compound D) do produce a transient inhibition of IR in both in vitro and in vivo models. Such transient inhibition of IR is thought to contribute to the anti-cancer efficacy of these molecules. Antibodies, which are typically more highly selective for IGF-1R, do not possess such an advantage. (d) Other small molecule IGF-1R kinase inhibitors (e.g. BMS-536924 (Bristol-Myers Squibb) inhibit both IGF-1R and IR in addition to a number of other kinases and are therefore less selective that IGF-1R kinase inhibitor compounds of Formula (I). This may contribute to the enhanced toxicity of these agents compared with IGF-1R kinase inhibitor compounds of Formula (I) (e.g. Compound D). Such toxicity may be even more pronounced when combined with other chemotherapies.

The present invention also provides a method for the treatment of cancer, comprising administering to a subject in need of such treatment an amount of an anti-cancer agent or treatment that elevates pAkt levels in tumor cells; and an amount of an IGF-1R kinase inhibitor of Formula (I); wherein at least one of the amounts is administered as a sub-therapeutic amount. In one embodiment, one or more other anti-cancer agents can additionally be administered to said patient.

The present invention also provides a method for treating tumors or tumor metastases in a patient, comprising administering to said patient simultaneously or sequentially a synergistically effective therapeutic amount of a combination of an anti-cancer agent or treatment that elevates pAkt levels in tumor cells and an IGF-1R kinase inhibitor of Formula (I). In one embodiment, one or more other anti-cancer agents can additionally be administered to said patient.

In embodiments of any of the methods of treatment of the invention described herein, the cells of the tumors or tumor metastases may be relatively insensitive or refractory to treatment with the anti-cancer agent or treatment as a single agent/treatment.

The present invention also provides a method for treating tumors or tumor metastases in a patient refractory to treatment with an anti-cancer agent or treatment that elevates pAkt levels in tumor cells as a single agent, comprising administering to said patient simultaneously or sequentially a therapeutically effective amount of a combination of said anti-cancer agent or treatment and an IGF-1R kinase inhibitor of Formula (I).

The present invention also provides a pharmaceutical composition comprising an anti-cancer agent or treatment that elevates pAkt levels in tumor cells and an IGF-1R kinase inhibitor of Formula (I), in a pharmaceutically acceptable carrier. In another embodiment, the pharmaceutical composition can additionally comprise one or more other anti-cancer agents.

The present invention also provides a kit comprising a container, comprising an IGF-1R kinase inhibitor of Formula (I), and an anti-cancer agent or treatment that elevates pAkt levels in tumor cells. In a preferred embodiment, the kit containers may further include a pharmaceutically acceptable carrier. The kit may further include a sterile diluent, which is preferably stored in a separate additional container. In another embodiment, the kit further comprising a package insert comprising printed instructions directing the use of a combined treatment of an IGF-1R kinase inhibitor of Formula (I) and the anti-cancer agent or treatment that elevates pAkt levels in tumor cells to a patient as a method for treating tumors, tumor metastases, or other cancers in a patient. The kit may also comprise additional containers comprising additional anti-cancer agents, agents that enhances the effect of such agents, or other compounds that improve the efficacy or tolerability of the treatment.

In any of the methods of treatment of the invention described herein the patient may be a patient in need of treatment for cancer, including, for example, NSCL, pancreatic, head and neck, colon, ovarian or breast cancers.

This invention also provides a method for treating abnormal cell growth of cells in a patient, comprising administering to said patient simultaneously or sequentially a therapeutically effective amount of a combination of an anti-cancer agent or treatment that elevates pAkt levels in tumor cells and an IGF-1R kinase inhibitor of Formula (I).

In one embodiment of the methods of this invention, the anti-cancer agent or treatment is administered at the same time as the IGF-1R kinase inhibitor of Formula (I). In another embodiment of the methods of this invention, the anti-cancer agent or treatment is administered prior to the IGF-1R kinase inhibitor of Formula (I). In another embodiment of the methods of this invention, the anti-cancer agent or treatment is administered after the IGF-1R kinase inhibitor of Formula (I). In another embodiment of the methods of this invention, the IGF-1R kinase inhibitor of Formula (I) is pre-administered prior to administration of a combination of IGF-1R kinase inhibitor of Formula (I) and the anti-cancer agent or treatment.

The present invention further provides a method for treating tumors or tumor metastases in a patient, comprising administering to said patient simultaneously or sequentially a therapeutically effective amount of a combination of an anti-cancer agent or treatment that elevates pAkt levels in tumor cells and an IGF-1R kinase inhibitor of Formula (I), and in addition, one or more other cytotoxic, chemotherapeutic or anti-cancer agents, or compounds that enhance the effects of such agents.

In the context of this invention, other cytotoxic, chemotherapeutic or anti-cancer agents, or compounds that enhance the effects of such agents, include, for example: alkylating agents or agents with an alkylating action, such as cyclophosphamide (CTX; e.g. CYTOXAN®, chlorambucil (CHL; e.g. LEUKERAN®), cisplatin (C is P; e.g. PLATINOL®) busulfan (e.g. MYLERAN®), melphalan, carmustine (BCNU), streptozotocin, triethylenemelamine (TEM), mitomycin C, and the like; anti-metabolites, such as methotrexate (MTX), etoposide (VP16; e.g. VEPESID®), 6-mercaptopurine (6 MP), 6-thiocguanine (6TG), cytarabine (Ara-C), 5-fluorouracil (5-FU), capecitabine (e.g. XELODA®), dacarbazine (DTIC), and the like; antibiotics, such as actinomycin D, doxorubicin (DXR; e.g. ADRIAMYCIN®), daunorubicin (daunomycin), bleomycin, mithramycin and the like; alkaloids, such as vinca alkaloids such as vincristine (VCR), vinblastine, and the like; and other antitumor agents, such as paclitaxel (e.g. TAXOL®) and pactitaxel derivatives, the cytostatic agents, glucocorticoids such as dexamethasone (DEX; e.g. DECADRON®) and corticosteroids such as prednisone, nucleoside enzyme inhibitors such as hydroxyurea, amino acid depleting enzymes such as asparaginase, leucovorin and other folic acid derivatives, and similar, diverse antitumor agents. The following agents may also be used as additional agents: arnifostine (e.g. ETHYOL®), dactinomycin, mechlorethamine (nitrogen mustard), streptozocin, cyclophosphamide, lomustine (CCNU), doxorubicin lipo (e.g. DOXIL®), gemcitabine (e.g. GEMZAR®), daunorubicin lipo (e.g. DAUNOXOME®), procarbazine, mitomycin, docetaxel (e.g. TAXOTERE®, aldesleukin, carboplatin, oxaliplatin, cladribine, camptothecin, CPT 11 (irinotecan), 10-hydroxy 7-ethyl-camptothecin (SN38), floxuridine, fludarabine, ifosfamide, idarubicin, mesna, interferon beta, interferon alpha, mitoxantrone, topotecan, leuprolide, megestrol, melphalan, mercaptopurine, plicamycin, mitotane, pegaspargase, pentostatin, pipobroman, plicamycin, tamoxifen, teniposide, testolactone, thioguanine, thiotepa, uracil mustard, vinorelbine, chlorambucil.

The present invention further provides a method for treating tumors or tumor metastases in a patient, comprising administering to said patient simultaneously or sequentially a therapeutically effective amount of a combination of an anti-cancer agent or treatment that elevates pAkt levels in tumor cells and an IGF-1R kinase inhibitor of Formula (I), and in addition, one or more anti-hormonal agents. As used herein, the term “anti-hormonal agent” includes natural or synthetic organic or peptidic compounds that act to regulate or inhibit hormone action on tumors.

Antihormonal agents include, for example: steroid receptor antagonists, anti-estrogens such as tamoxifen, raloxifene, aromatase inhibiting 4(5)-imidazoles, other aromatase inhibitors, 42-hydroxytamoxifen, trioxifene, keoxifene, LY 117018, onapristone, and toremifene (e.g. FARESTON®); anti-androgens such as flutamide, nilutamide, bicalutamide, leuprolide, and goserelin; and pharmaceutically acceptable salts, acids or derivatives of any of the above; agonists and/or antagonists of glycoprotein hormones such as follicle stimulating hormone (FSH), thyroid stimulating hormone (TSH), and luteinizing hormone (LH) and LHRH (leuteinizing hormone-releasing hormone); the LHRH agonist goserelin acetate, commercially available as ZOLADEX® (AstraZeneca); the LHRH antagonist D-alaninamide N-acetyl-3-(2-naphthalenyl)-D-alanyl-4-chloro-D-phenylalanyl-3-(3-pyridinyl)-D-alanyl-L-seryl-N6-(3-pyridinylcarbonyl)-L-lysyl-N6-(3-pyridinylcarbonyl)-D-lysyl-L-leucyl-N6-(1-methylethyl)-L-lysyl-L-proline (e.g ANTIDE®, Ares-Serono); the LHRH antagonist ganirelix acetate; the steroidal anti-androgens cyproterone acetate (CPA) and megestrol acetate, commercially available as MEGACEB® (Bristol-Myers Oncology); the nonsteroidal anti-androgen flutamide (2-methyl-N-[4, 20-nitro-3-(trifluoromethyl) phenylpropanamide), commercially available as EULEXIN® (Schering Corp.); the non-steroidal anti-androgen nilutamide, (5,5-dimethyl-3-[4-nitro-3-(trifluoromethyl-4′-nitrophenyl)-4,4-dimethyl-imidazolidine-dione); and antagonists for other non-permissive receptors, such as antagonists for RAR, RXR, TR, VDR, and the like.

The use of the cytotoxic and other anticancer agents described above in chemotherapeutic regimens is generally well characterized in the cancer therapy arts, and their use herein falls under the same considerations for monitoring tolerance and effectiveness and for controlling administration routes and dosages, with some adjustments. For example, the actual dosages of the cytotoxic agents may vary depending upon the patient's cultured cell response determined by using histoculture methods. Generally, the dosage will be reduced compared to the amount used in the absence of additional other agents.

Typical dosages of an effective cytotoxic agent can be in the ranges recommended by the manufacturer, and where indicated by in vitro responses or responses in animal models, can be reduced by up to about one order of magnitude concentration or amount. Thus, the actual dosage will depend upon the judgment of the physician, the condition of the patient, and the effectiveness of the therapeutic method based on the in vitro responsiveness of the primary cultured malignant cells or histocultured tissue sample, or the responses observed in the appropriate animal models.

The present invention further provides a method for treating tumors or tumor metastases in a patient, comprising administering to said patient simultaneously or sequentially a therapeutically effective amount of a combination of an anti-cancer agent or treatment that elevates pAkt levels in tumor cells and an IGF-1R kinase inhibitor of Formula (I), and in addition, one or more angiogenesis inhibitors.

Anti-angiogenic agents include, for example: VEGFR inhibitors, such as SU-5416 and SU-6668 (Sugen Inc. of South San Francisco, Calif., USA), or as described in, for example International Application Nos. WO 99/24440, WO 99/62890, WO 95/21613, WO 99/61422, WO 98/50356, WO 99/10349, WO 97/32856, WO 97/22596, WO 98/54093, WO 98/02438, WO 99/16755, and WO 98/02437, and U.S. Pat. Nos. 5,883,113, 5,886,020, 5,792,783, 5,834,504 and 6,235,764; VEGF inhibitors such as IM862 (Cytran Inc. of Kirkland, Wash., USA); angiozyme, a synthetic ribozyme from Ribozyme (Boulder, Colo.) and Chiron (Emeryville, Calif.); OSI-930 (OSI Pharmaceuticals, Melville, USA); and antibodies to VEGF, such as bevacizumab (e.g. AVASTIN™, Genentech, South San Francisco, Calif.), a recombinant humanized antibody to VEGF; integrin receptor antagonists and integrin antagonists, such as to αᵥβ₃, αᵥβ₅ and αᵥβ₆ integrins, and subtypes thereof, e.g. cilengitide (EMD 121974), or the anti-integrin antibodies, such as for example αᵥβ₃ specific humanized antibodies (e.g. VITAXIN®); factors such as IFN-alpha (U.S. Pat. Nos. 41,530,901, 4,503,035, and 5,231,176); angiostatin and plasminogen fragments (e.g. kringle 1-4, kringle 5, kringle 1-3 (O'Reilly, M. S. et al. (1994) Cell 79:315-328; Cao et al. (1996) J. Biol. Chem. 271: 29461-29467; Cao et al. (1997) J. Biol. Chem. 272:22924-22928); endostatin (O'Reilly, M. S. et al. (1997) Cell 88:277; and International Patent Publication No. WO 97/15666); thrombospondin (TSP-1; Frazier, (1991) Curr. Opin. Cell Biol. 3:792); platelet factor 4 (PF4); plasminogen activator/urokinase inhibitors; urokinase receptor antagonists; heparinases; fumagillin analogs such as TNP-4701; suramin and suramin analogs; angiostatic steroids; bFGF antagonists; flk-1 and flt-1 antagonists; anti-angiogenesis agents such as MMP-2 (matrix-metalloproteinase 2) inhibitors and MMP-9 (matrix-metalloproteinase 9) inhibitors. Examples of useful matrix metalloproteinase inhibitors are described in International Patent Publication Nos. WO 96/33172, WO 96/27583, WO 98/07697, WO 98/03516, WO 98/34918, WO 98/34915, WO 98/33768, WO 98/30566, WO 90/05719, WO 99/52910, WO 99/52889, WO 99/29667, and WO 99/07675, European Patent Publication Nos. 818,442, 780,386, 1,004,578, 606,046, and 931,788; Great Britain Patent Publication No. 9912961, and U.S. Pat. Nos. 5,863,949 and 5,861,510. Preferred MMP-2 and MMP-9 inhibitors are those that have little or no activity inhibiting MMP-1. More preferred, are those that selectively inhibit MMP-2 and/or MMP-9 relative to the other matrix-metalloproteinases (i.e. MMP-1, MMP-3, MMP-4, MMP-5, MMP-6, MMP-7, MMP-8, MMP-10, MMP-11, MMP-12, and MMP-13).

The present invention further provides a method for treating tumors or tumor metastases in a patient, comprising administering to said patient simultaneously or sequentially a therapeutically effective amount of a combination of an anti-cancer agent or treatment that elevates pAkt levels in tumor cells and an IGF-1R kinase inhibitor of Formula (I), and in addition, one or more other tumor cell pro-apoptotic or apoptosis-stimulating agents.

The present invention further provides a method for treating tumors or tumor metastases in a patient, comprising administering to said patient simultaneously or sequentially a therapeutically effective amount of a combination of an anti-cancer agent or treatment that elevates pAkt levels in tumor cells and an IGF-1R kinase inhibitor of Formula (I), and in addition, one or more other signal transduction inhibitors.

Signal transduction inhibitors include, for example: erbB2 receptor inhibitors, such as organic molecules, or antibodies that bind to the erbB2 receptor, for example, trastuzumab (e.g. HERCEPTIN®); inhibitors of other protein tyrosine-kinases, e.g. imitinib (e.g. GLEEVEC®); EGFR kinase inhibitors (see herein below); ras inhibitors; raf inhibitors; MEK inhibitors; mTOR inhibitors, including mTOR inhibitors that bind to and directly inhibits both mTORC1 and mTORC2 kinases; mTOR inhibitors that are dual PI3K/mTOR kinase inhibitors, such as for example the compound PI-103 as described in Fan, Q-W et al (2006) Cancer Cell 9:341-349 and Knight, Z. A. et al. (2006) Cell 125:733-747; mTOR inhibitors that are dual inhibitors of mTOR kinase and one or more other PIKK (or PIK-related) kinase family members. Such members include MEC1, TEL1, RAD3, MEI-41, DNA-PK, ATM, ATR, TRRAP, PI3K, and PI4K kinases; cyclin dependent kinase inhibitors; protein kinase C inhibitors; PI-3 kinase inhibitors; and PDK-1 inhibitors (see Dancey, J. and Sausville, E. A. (2003) Nature Rev. Drug Discovery 2:92-313, for a description of several examples of such inhibitors, and their use in clinical trials for the treatment of cancer).

ErbB2 receptor inhibitors include, for example: ErbB2 receptor inhibitors, such as GW-282974 (Glaxo Wellcome plc), monoclonal antibodies such as AR-209 (Aronex Pharmaceuticals Inc. of The Woodlands, Tex., USA) and 2B-1 (Chiron), and erbB2 inhibitors such as those described in International Publication Nos. WO 98/02434, WO 99/35146, WO 99/35132, WO 98/02437, WO 97/13760, and WO 95/19970, and U.S. Pat. Nos. 5,587,458, 5,877,305, 6,465,449 and 6,541,481.

As used herein, the term “mTOR inhibitor that binds to and directly inhibits both mTORC1 and mTORC2 kinases” refers to any mTOR inhibitor that binds to and directly inhibits both mTORC1 and mTORC2 kinases that is currently known in the art, or will be identified in the future, and includes any chemical entity that, upon administration to a patient, binds to and results in direct inhibition of both mTORC1 and mTORC2 kinases in the patient. Examples of mTOR inhibitors useful in the invention described herein include those disclosed and claimed in U.S. patent application Ser. No. 11/599,663, filed Nov. 15, 2006, a series of compounds that inhibit mTOR by binding to and directly inhibiting both mTORC1 and mTORC2 kinases.

As used herein, the term “EGFR kinase inhibitor” refers to any EGFR kinase inhibitor that is currently known in the art or that will be identified in the future, and includes any chemical entity that, upon administration to a patient, results in inhibition of a biological activity associated with activation of the EGF receptor in the patient, including any of the downstream biological effects otherwise resulting from the binding to EGFR of its natural ligand. Such EGFR kinase inhibitors include any agent that can block EGFR activation or any of the downstream biological effects of EGFR activation that are relevant to treating cancer in a patient. Such an inhibitor can act by binding directly to the intracellular domain of the receptor and inhibiting its kinase activity. Alternatively, such an inhibitor can act by occupying the ligand binding site or a portion thereof of the EGF receptor, thereby making the receptor inaccessible to its natural ligand so that its normal biological activity is prevented or reduced. Alternatively, such an inhibitor can act by modulating the dimerization of EGFR polypeptides, or interaction of EGFR polypeptide with other proteins, or enhance ubiquitination and endocytotic degradation of EGFR. EGFR kinase inhibitors include but are not limited to low molecular weight inhibitors, antibodies or antibody fragments, peptide or RNA aptamers, antisense constructs, small inhibitory RNAs (i.e. RNA interference by dsRNA; RNAi), and ribozymes. In a preferred embodiment, the EGFR kinase inhibitor is a small organic molecule or an antibody that binds specifically to the human EGFR.

EGFR kinase inhibitors include, for example quinazoline EGFR kinase inhibitors, pyrido-pyrimidine EGFR kinase inhibitors, pyrimido-pyrimidine EGFR kinase inhibitors, pyrrolo-pyrimidine EGFR kinase inhibitors, pyrazolo-pyrimidine EGFR kinase inhibitors, phenylamino-pyrimidine EGFR kinase inhibitors, oxindole EGFR kinase inhibitors, indolocarbazole EGFR kinase inhibitors, phthalazine EGFR kinase inhibitors, isoflavone EGFR kinase inhibitors, quinalone EGFR kinase inhibitors, and tyrphostin EGFR kinase inhibitors, such as those described in the following patent publications, and all pharmaceutically acceptable salts and solvates of said EGFR kinase inhibitors: International Patent Publication Nos. WO 96/33980, WO 96/30347, WO 97/30034, WO 97/30044, WO 97/38994, WO 97/49688, WO 98/02434, WO 97/38983, WO 95/19774, WO 95/19970, WO 97/13771, WO 98/02437, WO 98/02438, WO 97/32881, WO 98/33798, WO 97/32880, WO 97/3288, WO 97/02266, WO 97/27199, WO 98/07726, WO 97/34895, WO 96/31510, WO 98/14449, WO 98/14450, WO 98/14451, WO 95/09847, WO 97/19065, WO 98/17662, WO 99/35146, WO 99/35132, WO 99/07701, and WO 92/20642; European Patent Application Nos. EP 520722, EP 566226, EP 787772, EP 837063, and EP 682027; U.S. Pat. Nos. 5,747,498, 5,789,427, 5,650,415, and 5,656,643; and German Patent Application No. DE-19629652. Additional non-limiting examples of low molecular weight EGFR kinase inhibitors include any of the EGFR kinase inhibitors described in Traxler, P., 1998, Exp. Opin. Ther. Patents 8(12):1599-1625.

Specific preferred examples of low molecular weight EGFR kinase inhibitors that can be used according to the present invention include [6,7-bis(2-methoxyethoxy)-4-quinazolin-4-yl]-(3-ethynylphenyl) amine (also known as OSI-774, erlotinib, or TARCEVA® (erlotinib HCl); OSI Pharmaceuticals/Genentech/Roche) (U.S. Pat. No. 5,747,498; International Patent Publication No. WO 01/34574, and Moyer, J. D. et al. (1997) Cancer Res. 57:4838-4848); CI-1033 (formerly known as PD183805; Pfizer) (Sherwood et al., 1999, Proc. Am. Assoc. Cancer Res. 40:723); PD-158780 (Pfizer); AG-1478 (University of California); CGP-59326 (Novartis); PKI-166 (Novartis); EKB-569 (Wyeth); GW-2016 (also known as GW-572016 or lapatinib ditosylate; GSK); and gefitinib (also known as ZD1839 or IRESSA™; Astrazeneca) (Woodburn et al., 1997, Proc. Am. Assoc. Cancer Res. 38:633). A particularly preferred low molecular weight EGFR kinase inhibitor that can be used according to the present invention is [6,7-bis(2-methoxyethoxy)-4-quinazolin-4-yl]-(3-ethynylphenyl) amine (i.e. erlotinib), its hydrochloride salt (i.e. erlotinib HCl, TARCEVA®), or other salt forms (e.g. erlotinib mesylate).

EGFR kinase inhibitors also include, for example multi-kinase inhibitors that have activity on EGFR kinase, i.e. inhibitors that inhibit EGFR kinase and one or more additional kinases. Examples of such compounds include the EGFR and HER2 inhibitor CI-1033 (formerly known as PD183805; Pfizer); the EGFR and HER2 inhibitor GW-2016 (also known as GW-572016 or lapatinib ditosylate; GSK); the EGFR and JAK 2/3 inhibitor AG490 (a tyrphostin); the EGFR and HER2 inhibitor ARRY-334543 (Array BioPharma); BIBW-2992, an irreversible dual EGFR/HER2 kinase inhibitor (Boehringer Ingeiheim Corp.); the EGFR and HER2 inhibitor EKB-569 (Wyeth); the VEGF-R2 and EGFR inhibitor ZD6474 (also known as ZACTIMA™; AstraZeneca Pharmaceuticals), and the EGFR and HER2 inhibitor BMS-599626 (Bristol-Myers Squibb).

Antibody-based EGFR kinase inhibitors include any anti-EGFR antibody or antibody fragment that can partially or completely block EGFR activation by its natural ligand. Non-limiting examples of antibody-based EGFR kinase inhibitors include those described in Modjtahedi, H., et al., 1993, Br. J. Cancer 67:247-253; Teramoto, T., et al., 1996, Cancer 77:639-645; Goldstein et al., 1995, Clin. Cancer Res. 1:1311-1318; Huang, S. M., et al., 1999, Cancer Res. 15:59(8):1935-40; and Yang, X., et al., 1999, Cancer Res. 59:1236-1243. Thus, the EGFR kinase inhibitor can be the monoclonal antibody Mab E7.6.3 (Yang, X. D. et al. (1999) Cancer Res. 59:1236-43), or Mab C225 (ATCC Accession No. HB-8508), or an antibody or antibody fragment having the binding specificity thereof. Suitable monoclonal antibody EGFR kinase inhibitors include, but are not limited to, IMC-C225 (also known as cetuximab or ERBITUX™; Imclone Systems), ABX-EGF (Abgenix), EMD 72000 (Merck KgaA, Darmstadt), RH3 (York Medical Bioscience Inc.), and MDX-447 (Medarex/Merck KgaA).

EGFR kinase inhibitors for use in the present invention can alternatively be peptide or RNA aptamers. Such aptamers can for example interact with the extracellular or intracellular domains of EGFR to inhibit EGFR kinase activity in cells. An aptamer that interacts with the extracellular domain is preferred as it would not be necessary for such an aptamer to cross the plasma membrane of the target cell. An aptamer could also interact with the ligand for EGFR (e.g. EGF, TGF-α), such that its ability to activate EGFR is inhibited. Methods for selecting an appropriate aptamer are well known in the art. Such methods have been used to select both peptide and RNA aptamers that interact with and inhibit EGFR family members (e.g. see Buerger, C. et al. et al. (2003) J. Biol. Chem. 278:37610-37621; Chen, C-H. B. et al. (2003) Proc. Natl. Acad. Sci. 100:9226-9231; Buerger, C. and Groner, B. (2003) J. Cancer Res. Clin. Oncol. 129(12):669-675. Epub 2003 Sep. 11.).

EGFR kinase inhibitors for use in the present invention can alternatively be based on antisense oligonucleotide constructs. Anti-sense oligonucleotides, including anti-sense RNA molecules and anti-sense DNA molecules, would act to directly block the translation of EGFR mRNA by binding thereto and thus preventing protein translation or increasing mRNA degradation, thus decreasing the level of EGFR kinase protein, and thus activity, in a cell. For example, antisense oligonucleotides of at least about 15 bases and complementary to unique regions of the mRNA transcript sequence encoding EGFR can be synthesized, e.g., by conventional phosphodiester techniques and administered by e.g., intravenous injection or infusion. Methods for using antisense techniques for specifically inhibiting gene expression of genes whose sequence is known are well known in the art (e.g. see U.S. Pat. Nos. 6,566,135; 6,566,131; 6,365,354; 6,410,323; 6,107,091; 6,046,321; and 5,981,732).

Small inhibitory RNAs (siRNAs) can also function as EGFR kinase inhibitors for use in the present invention. EGFR gene expression can be reduced by contacting the tumor, subject or cell with a small double stranded RNA (dsRNA), or a vector or construct causing the production of a small double stranded RNA, such that expression of EGFR is specifically inhibited (i.e. RNA interference or RNAi). Methods for selecting an appropriate dsRNA or dsRNA-encoding vector are well known in the art for genes whose sequence is known (e.g. see Tuschi, T., et al. (1999) Genes Dev. 13(24):3191-3197; Elbashir, S. M. et al. (2001) Nature 411:494-498; Hannon, G. J. (2002) Nature 418:244-251; McManus, M. T. and Sharp, P. A. (2002) Nature Reviews Genetics 3:737-747; Bremmelkamp, T. R. et al. (2002) Science 296:550-553; U.S. Pat. Nos. 6,573,099 and 6,506,559; and International Patent Publication Nos. WO 01/36646, WO 99/32619, and WO 01/68836).

Ribozymes can also function as EGFR kinase inhibitors for use in the present invention. Ribozymes are enzymatic RNA molecules capable of catalyzing the specific cleavage of RNA. The mechanism of ribozyme action involves sequence specific hybridization of the ribozyme molecule to complementary target RNA, followed by endonucleolytic cleavage. Engineered hairpin or hammerhead motif ribozyme molecules that specifically and efficiently catalyze endonucleolytic cleavage of EGFR mRNA sequences are thereby useful within the scope of the present invention. Specific ribozyme cleavage sites within any potential RNA target are initially identified by scanning the target molecule for ribozyme cleavage sites, which typically include the following sequences, GUA, GUU, and GUC. Once identified, short RNA sequences of between about 15 and 20 ribonucleotides corresponding to the region of the target gene containing the cleavage site can be evaluated for predicted structural features, such as secondary structure, that can render the oligonucleotide sequence unsuitable. The suitability of candidate targets can also be evaluated by testing their accessibility to hybridization with complementary oligonucleotides, using, e.g., ribonuclease protection assays.

Both antisense oligonucleotides and ribozymes useful as EGFR kinase inhibitors can be prepared by known methods. These include techniques for chemical synthesis such as, e.g., by solid phase phosphoramadite chemical synthesis. Alternatively, anti-sense RNA molecules can be generated by in vitro or in vivo transcription of DNA sequences encoding the RNA molecule. Such DNA sequences can be incorporated into a wide variety of vectors that incorporate suitable RNA polymerase promoters such as the T7 or SP6 polymerase promoters. Various modifications to the oligonucleotides of the invention can be introduced as a means of increasing intracellular stability and half-life. Possible modifications include but are not limited to the addition of flanking sequences of ribonucleotides or deoxyribonucleotides to the 5′ and/or 3′ ends of the molecule, or the use of phosphorothioate- or 2′-O-methyl rather than phosphodiesterase linkages within the oligonucleotide backbone.

The present invention further provides a method for treating tumors or tumor metastases in a patient, comprising administering to said patient simultaneously or sequentially a therapeutically effective amount of a combination of an anti-cancer agent or treatment that elevates pAkt levels in tumor cells and an IGF-1R kinase inhibitor of Formula (I), and in addition, an anti-HER2 antibody or an immunotherapeutically active fragment thereof.

The present invention further provides a method for treating tumors or tumor metastases in a patient, comprising administering to said patient simultaneously or sequentially a therapeutically effective amount of a combination of an anti-cancer agent or treatment that elevates pAkt levels in tumor cells and an IGF-1R kinase inhibitor of Formula (I), and in addition, one or more additional anti-proliferative agents.

Additional antiproliferative agents include, for example: Inhibitors of the enzyme farnesyl protein transferase, PDGFR kinase inhibitors, including the compounds disclosed and claimed in U.S. Pat. Nos. 6,080,769, 6,194,438, 6,258,824, 6,586,447, 6,071,935, 6,495,564, 6,150,377, 6,596,735 and 6,479,513, and International Patent Publication WO 01/40217, IGF-1R kinase inhibitors other than IGF-1R kinase inhibitors of Formula (I), and FGFR kinase inhibitors.

As used herein, the term “PDGFR kinase inhibitor” refers to any PDGFR kinase inhibitor that is currently known in the art or that will be identified in the future, and includes any chemical entity that, upon administration to a patient, results in inhibition of a biological activity associated with activation of the PDGF receptor in the patient, including any of the downstream biological effects otherwise resulting from the binding to PDGFR of its natural ligand. Such PDGFR kinase inhibitors include any agent that can block PDGFR activation or any of the downstream biological effects of PDGFR activation that are relevant to treating cancer in a patient. Such an inhibitor can act by binding directly to the intracellular domain of the receptor and inhibiting its kinase activity. Alternatively, such an inhibitor can act by occupying the ligand binding site or a portion thereof of the PDGF receptor, thereby making the receptor inaccessible to its natural ligand so that its normal biological activity is prevented or reduced. Alternatively, such an inhibitor can act by modulating the dimerization of PDGFR polypeptides, or interaction of PDGFR polypeptide with other proteins, or enhance ubiquitination and endocytotic degradation of PDGFR. PDGFR kinase inhibitors include but are not limited to low molecular weight inhibitors, antibodies or antibody fragments, antisense constructs, small inhibitory RNAs (i.e. RNA interference by dsRNA; RNAi), and ribozymes. PDGFR kinase inhibitors include anti-PDGF or anti-PDGFR aptamers, anti-PDGF or anti-PDGFR antibodies, or soluble PDGF receptor decoys that prevent binding of a PDGF to its cognate receptor. In a preferred embodiment, the PDGFR kinase inhibitor is a small organic molecule or an antibody that binds specifically to the human PDGFR. The ability of a compound or agent to serve as a PDGFR kinase inhibitor may be determined according to the methods known in art and, further, as set forth in, e.g., Dai et al., (2001) Genes & Dev. 15: 1913-25; Zippel, et al., (1989) Eur. J. Cell Biol. 50(2):428-34; and Zwiller, et al., (1991) Oncogene 6: 219-21.

The invention includes PDGFR kinase inhibitors known in the art as well as those supported below and any and all equivalents that are within the scope of ordinary skill to create. For example, inhibitory antibodies directed against PDGF are known in the art, e.g., those described in U.S. Pat. Nos. 5,976,534, 5,833,986, 5,817,310, 5,882,644, 5,662,904, 5,620,687, 5,468,468, and PCT WO 2003/025019, the contents of which are incorporated by reference in their entirety. In addition, the invention includes N-phenyl-2-pyrimidine-amine derivatives that are PDGFR kinase inhibitors, such as those disclosed in U.S. Pat. No. 5,521,184, as well as WO2003/013541, WO2003/078404, WO2003/099771, WO2003/015282, and WO2004/05282 which are hereby incorporated in their entirety by reference.

Small molecules that block the action of PDGF are known in the art, e.g., those described in U.S. Patent or Published Application Nos. 6,528,526 (PDGFR tyrosine kinase inhibitors), 6,524,347 (PDGFR tyrosine kinase inhibitors), 6,482,834 (PDGFR tyrosine kinase inhibitors), 6,472,391 (PDGFR tyrosine kinase inhibitors), 6,949,563, 6,696,434, 6,331,555, 6,251,905, 6,245,760, 6,207,667, 5,990,141, 5,700,822, 5,618,837, 5,731,326, and 2005/0154014, and International Published Application Nos. WO 2005/021531, WO 2005/021544, and WO 2005/021537, the contents of which are incorporated by reference in their entirety.

Proteins and polypeptides that block the action of PDGF are known in the art, e.g., those described in U.S. Pat. Nos. 6,350,731 (PDGF peptide analogs), 5,952,304, the contents of which are incorporated by reference in their entirety.

Bis mono- and bicyclic aryl and heteroaryl compounds which inhibit EGF and/or PDGF receptor tyrosine kinase are known in the art, e.g., those described in, e.g. U.S. Pat. Nos. 5,476,851, 5,480,883, 5,656,643, 5,795,889, and 6,057,320, the contents of which are incorporated by reference in their entirety.

Antisense oligonucleotides for the inhibition of PDGF are known in the art, e.g., those described in U.S. Pat. Nos. 5,869,462, and 5,821,234, the contents of each of which are incorporated by reference in their entirety.

Aptamers (also known as nucleic acid ligands) for the inhibition of PDGF are known in the art, e.g., those described in, e.g., U.S. Pat. Nos. 6,582,918, 6,229,002, 6,207,816, 5,668,264, 5,674,685, and 5,723,594, the contents of each of which are incorporated by reference in their entirety.

Other compounds for inhibiting PDGF known in the art include those described in U.S. Pat. Nos. 5,238,950, 5,418,135, 5,674,892, 5,693,610, 5,700,822, 5,700,823, 5,728,726, 5,795,910, 5,817,310, 5,872,218, 5,932,580, 5,932,602, 5,958,959, 5,990,141, 6,358,954, 6,537,988 and 6,673,798, the contents of each of which are incorporated by reference in their entirety.

A number of types of tyrosine kinase inhibitors that are selective for tyrosine kinase receptor enzymes such as PDGFR are known (see, e.g., Spada and Myers ((1995) Exp. Opin. Ther. Patents, 5: 805) and Bridges ((1995) Exp. Opin. Ther. Patents, 5: 1245). Additionally Law and Lydon have summarized the anticancer potential of tyrosine kinase inhibitors ((1996) Emerging Drugs: The Prospect For Improved Medicines, 241-260). For example, U.S. Pat. No. 6,528,526 describes substituted quinoxaline compounds that selectively inhibit platelet-derived growth factor-receptor (PDGFR) tyrosine kinase activity. The known inhibitors of PDGFR tyrosine kinase activity includes quinoline-based inhibitors reported by Maguire et al., ((1994) J. Med. Chem., 37: 2129), and by Dolle, et al., ((1994) J. Med. Chem., 37: 2627). A class of phenylamino-pyrimidine-based inhibitors was recently reported by Traxler, et al., in EP 564409 and by Zimmerman et al., ((1996) Biorg. Med. Chem. Lett., 6: 1221-1226) and by Buchdunger, et al., ((1995) Proc. Nat. Acad. Sci. (USA), 92: 2558). Quinazoline derivatives that are useful in inhibiting PDGF receptor tyrosine kinase activity include bismono- and bicyclic aryl compounds and heteroaryl compounds (see, e.g., WO 92/20642), quinoxaline derivatives (see (1994) Cancer Res., 54: 6106-6114), pyrimidine derivatives (Japanese Published Patent Application No. 87834/94) and dimethoxyquinoline derivatives (see Abstracts of the 116th Annual Meeting of the Pharmaceutical Society of Japan (Kanazawa), (1996), 2, p. 275, 29(C2) 15-2).

Specific preferred examples of low molecular weight PDGFR kinase inhibitors that can be used according to the present invention include Imatinib (GLEEVEC®; Novartis); SU-12248 (sunitib malate, SUTENT®; Pfizer); Dasatinib (SPRYCEL®; BMS; also known as BMS-354825); Sorafenib (NEXAVAR®; Bayer; also known as Bay-43-9006); AG-13736 (Axitinib; Pfizer); RPR127963 (Sanofi-Aventis); CP-868596 (Pfizer/OSI Pharmaceuticals); MLN-518 (tandutinib; Millennium Pharmaceuticals); AMG-706 (Motesanib; Amgen); ARAVA® (leflunomide; Sanofi-Aventis; also known as SU101), and OSI-930 (OSI Pharmaceuticals); Additional preferred examples of low molecular weight PDGFR kinase inhibitors that are also FGFR kinase inhibitors that can be used according to the present invention include XL-999 (Exelixis); SU6668 (Pfizer); CHIR-258/TKI-258 (Chiron); RO4383596 (Hoffmann-La Roche) and BIBF-1120 (Boehringer Ingelheim).

As used herein, the term “IGF-1R kinase inhibitors other than IGF-1R kinase inhibitor of Formula (I)” refers to any IGF-1R kinase inhibitor, other than IGF-1R kinase inhibitor of Formula (I), that is currently known in the art or that will be identified in the future, and includes any chemical entity that, upon administration to a patient, results in inhibition of a biological activity associated with activation of the IGF-1 receptor in the patient, including any of the downstream biological effects otherwise resulting from the binding to IGF-1R of its natural ligand. Such IGF-1R kinase inhibitors include any agent that can block IGF-1R activation or any of the downstream biological effects of IGF-1R activation that are relevant to treating cancer in a patient. Such an inhibitor can act by binding directly to the intracellular domain of the receptor and inhibiting its kinase activity. Alternatively, such an inhibitor can act by occupying the ligand binding site or a portion thereof of the IGF-1 receptor, thereby making the receptor inaccessible to its natural ligand so that its normal biological activity is prevented or reduced. Alternatively, such an inhibitor can act by modulating the dimerization of IGF-1R polypeptides, or interaction of IGF-1R polypeptide with other proteins, or enhance ubiquitination and endocytotic degradation of IGF-1R. An IGF-1R kinase inhibitor can also act by reducing the amount of IGF-1 available to activate IGF-1R, by for example antagonizing the binding of IGF-1 to its receptor, by reducing the level of IGF-1, or by promoting the association of IGF-1 with proteins other than IGF-1R such as IGF binding proteins (e.g. IGFBP3). IGF-1R kinase inhibitors include but are not limited to low molecular weight inhibitors, antibodies or antibody fragments, antisense constructs, small inhibitory RNAs (i.e. RNA interference by dsRNA; RNAi), and ribozymes. In a preferred embodiment, the IGF-1R kinase inhibitor is a small organic molecule or an antibody that binds specifically to the human IGF-1R.

IGF-1R kinase inhibitors other than IGF-1R kinase inhibitor of Formula (I) include, for example imidazopyrazine IGF-1R kinase inhibitors, quinazoline IGF-1R kinase inhibitors, pyrido-pyrimidine IGF-1R kinase inhibitors, pyrimido-pyrimidine IGF-1R kinase inhibitors, pyrrolo-pyrimidine IGF-1R kinase inhibitors, pyrazolo-pyrimidine IGF-1R kinase inhibitors, phenylamino-pyrimidine IGF-1R kinase inhibitors, oxindole IGF-1R kinase inhibitors, indolocarbazole IGF-1R kinase inhibitors, phthalazine IGF-1R kinase inhibitors, isoflavone IGF-1R kinase inhibitors, quinalone IGF-1R kinase inhibitors, and tyrphostin IGF-1R kinase inhibitors, and all pharmaceutically acceptable salts and solvates of such IGF-1R kinase inhibitors.

Examples of IGF-1R kinase inhibitors other than IGF-1R kinase inhibitor of Formula (I) include those in International Patent Publication No. WO 05/037836, that describes imidazopyrazine IGF-1R kinase inhibitors, International Patent Publication Nos. WO 03/018021 and WO 03/018022, that describe pyrimidines for treating IGF-1R related disorders, International Patent Publication Nos. WO 02/102804 and WO 02/102805, that describe cyclolignans and cyclolignans as IGF-1R inhibitors, International Patent Publication No. WO 02/092599, that describes pyrrolopyrimidines for the treatment of a disease which responds to an inhibition of the IGF-1R tyrosine kinase, International Patent Publication No. WO 01/72751, that describes pyrrolopyrimidines as tyrosine kinase inhibitors, and in International Patent Publication No. WO 00/71129, that describes pyrrolotriazine inhibitors of kinases, and in International Patent Publication No. WO 97/28161, that describes pyrrolo[2,3-d]pyrimidines and their use as tyrosine kinase inhibitors, Parrizas, et al., which describes tyrphostins with in vitro and in vivo IGF-1R inhibitory activity (Endocrinology, 138:1427-1433 (1997)), International Patent Publication No. WO 00/35455, that describes heteroaryl-aryl ureas as IGF-1R inhibitors, International Patent Publication No. WO 03/048133, that describes pyrimidine derivatives as modulators of IGF-1R, International Patent Publication No. WO 03/024967, WO 03/035614, WO 03/035615, WO 03/035616, and WO 03/035619, that describe chemical compounds with inhibitory effects towards kinase proteins, International Patent Publication No. WO 03/068265, that describes methods and compositions for treating hyperproliferative conditions, International Patent Publication No. WO 00/17203, that describes pyrrolopyrimidines as protein kinase inhibitors, Japanese Patent Publication No. JP 07/133,280, that describes a cephem compound, its production and antimicrobial composition, Albert, A. et al., Journal of the Chemical Society, 11: 1540-1547 (1970), which describes pteridine studies and pteridines unsubstituted in the 4-position, and A. Albert et al., Chem. Biol. Pteridines Proc. Int. Symp., 4th, 4: 1-5 (1969) which describes a synthesis of pteridines (unsubstituted in the 4-position) from pyrazines, via 3-4-dihydropteridines.

Additional, specific examples of IGF-1R kinase inhibitors other than IGF-1R kinase inhibitor of Formula (I) that can be used according to the present invention include h7C10 (Centre de Recherche Pierre Fabre), an IGF-1 antagonist; EM-164 (ImmunoGen Inc.), an IGF-1R modulator; CP-751871 (Pfizer Inc.), an IGF-1 antagonist; lanreotide (Ipsen), an IGF-1 antagonist; IGF-1R oligonucleotides (Lynx Therapeutics Inc.); IGF-1 oligonucleotides (National Cancer Institute); IGF-1R protein-tyrosine kinase inhibitors in development by Novartis (e.g. NVP-AEW541, Garcia-Echeverria, C. et al. (2004) Cancer Cell 5:231-239; or NVP-ADW742, Mitsiades, C. S. et al. (2004) Cancer Cell 5:221-230); IGF-1R protein-tyrosine kinase inhibitors (Ontogen Corp); AG-1024 (Camirand, A. et al. (2005) Breast Cancer Research 7:R570-R579 (DOI 10.1186/bcr1028); Camirand, A. and Pollak, M. (2004) Brit. J. Cancer 90:1825-1829; Pfizer Inc.), an IGF-1 antagonist; the tyrphostins-AG-538 and I-OMe-AG 538; BMS-536924, a small molecule inhibitor of IGF-1R; PNU-145156E (Pharmacia & Upjohn SpA), an IGF-1 antagonist; BMS 536924, a dual IGF-1R and IR kinase inhibitor (Bristol-Myers Squibb); AEW541 (Novartis); GSK621659A (Glaxo Smith-Kline); INSM-18 (Insmed); and XL-228 (Exelixis).

Antibody-based IGF-1R kinase inhibitors include any anti-IGF-1R antibody or antibody fragment that can partially or completely block IGF-1R activation by its natural ligand. Antibody-based IGF-1R kinase inhibitors also include any anti-IGF-1 antibody or antibody fragment that can partially or completely block IGF-1R activation. Non-limiting examples of antibody-based IGF-1R kinase inhibitors include those described in Larsson, O. et al (2005) Brit. J. Cancer 92:2097-2101 and Ibrahim, Y. H. and Yee, D. (2005) Clin. Cancer Res. 11:944s-950s; or being developed by Imclone (e.g. IMC-A12), or AMG-479, an anti-IGF-1R antibody (Amgen); R1507, an anti-IGF-1R antibody (Genmab/Roche); AVE-1642, an anti-IGF-1R antibody (Immunogen/Sanofi-Aventis); MK 0646 or h7C10, an anti-IGF-1R antibody (Merck); or antibodies being develop by Schering-Plough Research Institute (e.g. SCH 717454 or 19D12; or as described in US Patent Application Publication Nos. US 2005/0136063 A1 and US 2004/0018191 A1). The IGF-1R kinase inhibitor can be a monoclonal antibody, or an antibody or antibody fragment having the binding specificity thereof.

As used herein, the term “FGFR kinase inhibitor” refers to any FGFR kinase inhibitor that is currently known in the art or that will be identified in the future, and includes any chemical entity that, upon administration to a patient, results in inhibition of a biological activity associated with activation of the FGF receptor in the patient, including any of the downstream biological effects otherwise resulting from the binding to FGFR of its natural ligand. Such FGFR kinase inhibitors include any agent that can block FGFR activation or any of the downstream biological effects of FGFR activation that are relevant to treating cancer in a patient. Such an inhibitor can act by binding directly to the intracellular domain of the receptor and inhibiting its kinase activity. Alternatively, such an inhibitor can act by occupying the ligand binding site or a portion thereof of the FGF receptor, thereby making the receptor inaccessible to its natural ligand so that its normal biological activity is prevented or reduced. Alternatively, such an inhibitor can act by modulating the dimerization of FGFR polypeptides, or interaction of FGFR polypeptide with other proteins, or enhance ubiquitination and endocytotic degradation of FGFR. FGFR kinase inhibitors include but are not limited to low molecular weight inhibitors, antibodies or antibody fragments, antisense constructs, small inhibitory RNAs (i.e. RNA interference by dsRNA; RNAi), and ribozymes. FGFR kinase inhibitors include anti-FGF or anti-FGFR aptamers, anti-FGF or anti-FGFR antibodies, or soluble FGFR receptor decoys that prevent binding of a FGFR to its cognate receptor. In a preferred embodiment, the FGFR kinase inhibitor is a small organic molecule or an antibody that binds specifically to the human FGFR. Anti-FGFR antibodies include FR1-H7 (FGFR-1) and FR3-D 11 (FGFR-3) (Imclone Systems, Inc.).

FGFR kinase inhibitors also include compounds that inhibit FGFR signal transduction by affecting the ability of heparan sulfate proteoglycans to modulate FGFR activity. Heparan sulfate proteoglycans in the extracellular matrix can mediate the actions of FGF, e.g., protection from proteolysis, localization, storage, and internalization of growth factors (Faham, S. et al. (1998) Curr. Opin. Struct. Biol., 8:578-586), and may serve as low affinity FGF receptors that act to present FGF to its cognate FGFR, and/or to facilitate receptor oligomerization (Galzie, Z. et al. (1997) Biochem. Cell. Biol., 75:669-685).

The invention includes FGFR kinase inhibitors known in the art (e.g. PD173074) as well as those supported below and any and all equivalents that are within the scope of ordinary skill to create.

Examples of chemicals that may antagonize FGF action, and can thus be used as FGFR kinase inhibitors in the methods described herein, include suramin, structural analogs of suramin, pentosan polysulfate, scopolamine, angiostatin, sprouty, estradiol, carboxymethylbenzylamine dextran (CMDB7), suradista, insulin-like growth factor binding protein-3, ethanol, heparin (e.g., 6-O-desulfated heparin), low molecular weight heparin, protamine sulfate, cyclosporin A, or RNA ligands for bFGF.

Other agents or compounds for inhibiting FGFR kinase known in the art include those described in U.S. Pat. Nos. 7,151,176 (Bristol-Myers Squibb Company; Pyrrolotriazine compounds); 7,102,002 (Bristol-Myers Squibb Company; pyrrolotriazine compounds); 5,132,408 (Salk Institute; peptide FGF antagonists); and 5,945,422 (Warner-Lambert Company; 2-amino-substituted pyrido[2,3-d]pyrimidines); U.S. published Patent application Nos. 2005/0256154 (4-amino-thieno[3,2-c]pyridine-7-carboxylic acid amide compounds); and 2004/0204427 (pyrimidino compounds); and published International Patent Applications WO-2007019884 (Merck Patent GmbH; N-(3-pyrazolyl)-N′-4-(4-pyridinyloxy)phenyl)urea compounds); WO-2007009773 (Novartis AG; pyrazolo[1,5-a]pyrimidin-7-yl amine derivatives); WO-2007014123 (Five Prime Therapeutics, Inc.; FGFR fusion proteins); WO-2006134989 (Kyowa Hakko Kogyo Co., Ltd.; nitrogenous heterocycle compounds); WO-2006112479 (Kyowa Hakko Kogyo Co., Ltd.; azaheterocycles); WO-2006108482 (Merck Patent GmbH; 9-(4-ureidophenyl)purine compounds); WO-2006105844 (Merck Patent GmbH; N-(3-pyrazolyl)-N′-4-(4-pyridinyloxy)phenyl)urea compounds); WO-2006094600 (Merck Patent GmbH; tetrahydropyrroloquinoline derivatives); WO-2006050800 (Merck Patent GmbH; N,N′-diarylurea derivatives); WO-2006050779 (Merck Patent GmbH; N,N′-diarylurea derivatives); WO-2006042599 (Merck Patent GmbH; phenylurea derivatives); WO-2005066211 (Five Prime Therapeutics, Inc.; anti-FGFR antibodies); WO-2005054246 (Merck Patent GmbH; heterocyclyl amines); WO-2005028448 (Merck Patent GmbH; 2-amino-1-benzyl-substituted benzimidazole derivatives); WO-2005011597 (Irm Llc; substituted heterocyclic derivatives); WO-2004093812 (Irm Llc/Scripps; 6-phenyl-7H-pyrrolo[2,3-d]pyrimidine derivatives); WO-2004046152 (F. Hoffmann La Roche AG; pyrimido[4,5-e]oxadiazine derivatives); WO-2004041822 (F. Hoffmann La Roche AG; pyrimido[4,5-d]pyrimidine derivatives); WO-2004018472 (F. Hoffmann La Roche AG; pyrimido[4,5-d]pyrimidine derivatives); WO-2004013145 (Bristol-Myers Squibb Company; pyrrolotriazine derivatives); WO-2004009784 (Bristol-Myers Squibb Company; pyrrolo[2,1-f][1,2,4]triazin-6-yl compounds); WO-2004009601 (Bristol-Myers Squibb Company; azaindole compounds); WO-2004001059 (Bristol-Myers Squibb Company; heterocyclic derivatives); WO-02102972 (Prochon Biotech Ltd./Morphosys AG; anti-FGFR antibodies); WO-02102973 (Prochon Biotech Ltd.; anti-FGFR antibodies); WO-00212238 (Warner-Lambert Company; 2-(pyridin-4-ylamino)-6-dialkoxyphenyl-pyrido[2,3-d]pyrimidin-7-one derivatives); WO-00170977 (Amgen, Inc.; FGFR-L and derivatives); WO-00132653 (Cephalon, Inc.; pyrazolone derivatives); WO-00046380 (Chiron Corporation; FGFR-Ig fusion proteins); and WO-00015781 (Eli Lilly; polypeptides related to the human SPROUTY-1 protein).

Specific preferred examples of low molecular weight FGFR kinase inhibitors that can be used according to the present invention include RO-4396686 (Hoffmann-La Roche); CHIR-258 (Chiron; also known as TKI-258); PD 173074 (Pfizer); PD 166866 (Pfizer); ENK-834 and ENK-835 (both Enkam Pharmaceuticals A/S); and SU5402 (Pfizer). Additional preferred examples of low molecular weight FGFR kinase inhibitors that are also PDGFR kinase inhibitors that can be used according to the present invention include XL-999 (Exelixis); SU6668 (Pfizer); CHIR-258/TKI-258 (Chiron); R04383596 (Hoffmann-La Roche), and BIBF-1120 (Boehringer Ingelheim).

The present invention further provides a method for treating tumors or tumor metastases in a patient, comprising administering to said patient simultaneously or sequentially a therapeutically effective amount of a combination of an anti-cancer agent or treatment that elevates pAkt levels in tumor cells and an IGF-1R kinase inhibitor of Formula (I), and in addition, a COX II (cyclooxygenase II) inhibitor. Examples of useful COX-II inhibitors include alecoxib (e.g. CELEBREX™) and valdecoxib (e.g. BEXTRA™).

The present invention further provides a method for treating tumors or tumor metastases in a patient, comprising administering to said patient simultaneously or sequentially a therapeutically effective amount of a combination of an anti-cancer agent or treatment that elevates pAkt levels in tumor cells and an IGF-1R kinase inhibitor of Formula (I), and in addition treatment with radiation or a radiopharmaceutical.

The source of radiation can be either external or internal to the patient being treated. When the source is external to the patient, the therapy is known as external beam radiation therapy (EBRT). When the source of radiation is internal to the patient, the treatment is called brachytherapy (BT). Radioactive atoms for use in the context of this invention can be selected from the group including, but not limited to, radium, cesium-137, iridium-192, americium-241, gold-198, cobalt-57, copper-67, technetium-99, iodine-123, iodine-131, and indium-111.

Radiation therapy is a standard treatment for controlling unresectable or inoperable tumors and/or tumor metastases. Improved results have been seen when radiation therapy has been combined with chemotherapy. Radiation therapy is based on the principle that high-dose radiation delivered to a target area will result in the death of reproductive cells in both tumor and normal tissues. The radiation dosage regimen is generally defined in terms of radiation absorbed dose (Gy), time and fractionation, and must be carefully defined by the oncologist. The amount of radiation a patient receives will depend on various considerations, but the two most important are the location of the tumor in relation to other critical structures or organs of the body, and the extent to which the tumor has spread. A typical course of treatment for a patient undergoing radiation therapy will be a treatment schedule over a 1 to 6 week period, with a total dose of between 10 and 80 Gy administered to the patient in a single daily fraction of about 1.8 to 2.0 Gy, 5 days a week. Parameters of adjuvant radiation therapies are, for example, contained in International Patent Publication WO 99/60023.

The present invention further provides a method for treating tumors or tumor metastases in a patient, comprising administering to said patient simultaneously or sequentially a therapeutically effective amount of a combination of an anti-cancer agent or treatment that elevates pAkt levels in tumor cells and an IGF-1R kinase inhibitor of Formula (I), and in addition treatment with one or more agents capable of enhancing antitumor immune responses.

Agents capable of enhancing antitumor immune responses include, for example: CTLA4 (cytotoxic lymphocyte antigen 4) antibodies (e.g. MDX-CTLA4), and other agents capable of blocking CTLA4. Specific CTLA4 antibodies that can be used in the present invention include those described in U.S. Pat. No. 6,682,736.

The present invention further provides a method for reducing the side effects caused by the treatment of tumors or tumor metastases in a patient with an anti-cancer agent or treatment that elevates pAkt levels in tumor cells, comprising administering to said patient simultaneously or sequentially a therapeutically effective amount of a combination of an anti-cancer agent or treatment that elevates pAkt levels in tumor cells and an IGF-1R kinase inhibitor of Formula (I), in amounts that are effective to produce a superadditive or synergistic antitumor effect, and that are effective at inhibiting the growth of the tumor.

The present invention further provides a method for the treatment of cancer, comprising administering to a subject in need of such treatment (i) an effective first amount of an anti-cancer agent or treatment that elevates pAkt levels in tumor cells; and (ii) an effective second amount of an agent that sensitizes tumor cells to the effects of the anti-cancer agent or treatment, wherein that agent is an IGF-1R kinase inhibitor of Formula (I).

The present invention further provides a method for the treatment of cancer, comprising administering to a subject in need of such treatment (i) a sub-therapeutic first amount of an anti-cancer agent or treatment that elevates pAkt levels in tumor cells; and (ii) a sub-therapeutic second amount of an agent that sensitizes tumor cells to the effects of the anti-cancer agent or treatment, wherein that agent is an IGF-1R kinase inhibitor of Formula (I).

The present invention further provides a method for the treatment of cancer, comprising administering to a subject in need of such treatment (i) an effective first amount of an anti-cancer agent or treatment that elevates pAkt levels in tumor cells; and (ii) a sub-therapeutic second amount of an agent that sensitizes tumor cells to the effects of the anti-cancer agent or treatment, wherein that agent is an IGF-1R kinase inhibitor of Formula (I).

The present invention further provides a method for the treatment of cancer, comprising administering to a subject in need of such treatment (i) a sub-therapeutic first amount of an anti-cancer agent or treatment that elevates pAkt levels in tumor cells; and (ii) an effective second amount of an agent that sensitizes tumor cells to the effects of the anti-cancer agent or treatment, wherein that agent is an IGF-1R kinase inhibitor of Formula (I).

In the preceding methods the order of administration of the first and second amounts can be simultaneous or sequential, i.e. the agent that sensitizes tumor cells to the effects of the anti-cancer agent or treatment can be administered before the anti-cancer agent or treatment, after the anti-cancer agent or treatment, or at the same time as the anti-cancer agent or treatment.

In the context of this invention, an “effective amount” of an agent or therapy is as defined above. A “sub-therapeutic amount” of an agent or therapy is an amount less than the effective amount for that agent or therapy, but when combined with an effective or sub-therapeutic amount of another agent or therapy can produce a result desired by the physician, due to, for example, synergy in the resulting efficacious effects, or reduced side effects.

As used herein, the term “patient” preferably refers to a human in need of treatment with an anti-cancer agent or treatment for any purpose, and more preferably a human in need of such a treatment to treat cancer, or a precancerous condition or lesion. However, the term “patient” can also refer to non-human animals, preferably mammals such as dogs, cats, horses, cows, pigs, sheep and non-human primates, among others, that are in need of treatment with an anti-cancer agent or treatment.

In a preferred embodiment, the patient is a human in need of treatment for cancer, or a precancerous condition or lesion, wherein the cancer is preferably NSCL, pancreatic, head and neck, colon, ovarian or breast cancers, or Ewing's sarcoma. However, cancers that may be treated by the methods described herein include lung cancer, bronchioloalveolar cell lung cancer, bone cancer, skin cancer, cancer of the head or neck, cutaneous or intraocular melanoma, uterine cancer, ovarian cancer, rectal cancer, cancer of the anal region, stomach cancer, gastric cancer, uterine cancer, carcinoma of the fallopian tubes, carcinoma of the endometrium, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, Ewing's saccoma, cancer of the urethra, cancer of the penis, prostate cancer, cancer of the bladder, cancer of the ureter, carcinoma of the renal pelvis, mesothelioma, hepatocellular cancer, biliary cancer, cancer of the kidney, renal cell carcinoma, chronic or acute leukemia, lymphocytic lymphomas, neoplasms of the central nervous system (CNS), spinal axis tumors, brain stem glioma, glioblastoma multiforme, astrocytomas, schwannomas, ependymomas, medulloblastomas, meningiomas, squamous cell carcinomas, pituitary adenomas, including refractory versions of any of the above cancers, or a combination of one or more of the above cancers. The precancerous condition or lesion includes, for example, the group consisting of oral leukoplakia, actinic keratosis (solar keratosis), precancerous polyps of the colon or rectum, gastric epithelial dysplasia, adenomatous dysplasia, hereditary nonpolyposis colon cancer syndrome (HNPCC), Barrett's esophagus, bladder dysplasia, and precancerous cervical conditions.

The term “refractory” as used herein is used to define a cancer for which treatment (e.g. chemotherapy drugs, biological agents, and/or radiation therapy) has proven to be ineffective. A refractory cancer tumor may shrink, but not to the point where the treatment is determined to be effective. Typically however, the tumor stays the same size as it was before treatment (stable disease), or it grows (progressive disease).

For purposes of the present invention, “co-administration of” and “co-administering” an anti-cancer agent or treatment that elevates pAkt levels in tumor cells and an IGF-1R kinase inhibitor of Formula (I) (both components referred to hereinafter as the “two active agents”) refer to any administration of the two active agents, either separately or together, where the two active agents are administered as part of an appropriate dose regimen designed to obtain the benefit of the combination therapy. Thus, the two active agents can be administered either as part of the same pharmaceutical composition or in separate pharmaceutical compositions. The IGF-1R kinase inhibitor of Formula (I) that sensitizes tumor cells to the pro-apoptotic effects of the anti-cancer agent or treatment that elevates pAkt levels in tumor cells can be administered prior to, at the same time as, or subsequent to administration of the anti-cancer agent or treatment, or in some combination thereof. Where the anti-cancer agent or treatment is administered to the patient at repeated intervals, e.g., during a standard course of treatment, the IGF-1R kinase inhibitor of Formula (I) that sensitizes tumor cells to the effects of the anti-cancer agent or treatment can be administered prior to, at the same time as, or subsequent to, each administration of the anti-cancer agent or treatment, or some combination thereof, or at different intervals in relation to therapy with the anti-cancer agent or treatment, or in a single dose prior to, at any time during, or subsequent to the course of treatment with the anti-cancer agent or treatment.

The anti-cancer agent or treatment will typically be administered to the patient in a dose regimen that provides for the most effective treatment of the cancer (from both efficacy and safety perspectives) for which the patient is being treated, as known in the art. In conducting the treatment method of the present invention, the anti-cancer agent or treatment can be administered in any effective manner known in the art, such as by oral, topical, intravenous, intra-peritoneal, intramuscular, intra-articular, subcutaneous, intranasal, intra-ocular, vaginal, rectal, or intradermal routes, depending upon the type of cancer being treated, the type of anti-cancer agent or treatment being used, and the medical judgement of the prescribing physician as based, e.g., on the results of published clinical studies. When the anti-cancer agent or treatment is radiation or a radiochemical, the agent or treatment can be administered in any effective manner known in the art, as described briefly herein, above.

The amount of anti-cancer agent or treatment administered and the timing of anti-cancer agent or treatment administration will depend on the type (species, gender, age, weight, etc.) and condition of the patient being treated, the severity of the disease or condition being treated, and on the route of administration. In some instances, dosage levels below the lower limit of the aforesaid range may be more than adequate, while in other cases still larger doses may be employed without causing any harmful side effect, provided that such larger doses are first divided into several small doses for administration throughout the day.

The anti-cancer agent or treatment and the IGF-1R kinase inhibitor of Formula (I) that sensitizes tumor cells to the pro-apoptotic effects of the anti-cancer agent or treatment can be administered with various pharmaceutically acceptable inert carriers in the form of tablets, capsules, lozenges, troches, hard candies, powders, sprays, creams, salves, suppositories, jellies, gels, pastes, lotions, ointments, elixirs, syrups, and the like. Administration of such dosage forms can be carried out in single or multiple doses. Carriers include solid diluents or fillers, sterile aqueous media and various non-toxic organic solvents, etc. Oral pharmaceutical compositions can be suitably sweetened and/or flavored.

The anti-cancer agent or treatment and the IGF-1R kinase inhibitor of Formula (I) that sensitizes tumor cells to the pro-apoptotic effects of the anti-cancer agent or treatment can be combined together with various pharmaceutically acceptable inert carriers in the form of sprays, creams, salves, suppositories, jellies, gels, pastes, lotions, ointments, and the like. Administration of such dosage forms can be carried out in single or multiple doses. Carriers include solid diluents or fillers, sterile aqueous media, and various non-toxic organic solvents, etc.

Methods of preparing pharmaceutical compositions comprising anti-cancer agents or treatments are known in the art. Methods of preparing pharmaceutical compositions comprising IGF-1R kinase inhibitor of Formula (I) are also known in the art (e.g. US Published Patent Application 2006/0235031). In view of the teaching of the present invention, methods of preparing pharmaceutical compositions comprising both an anti-cancer agent or treatment and an IGF-1R kinase inhibitor of Formula (I) that sensitizes tumor cells to the pro-apoptotic effects of the anti-cancer agent or treatment will be apparent from the art, from other known standard references, such as Remington's Pharmaceutical Sciences, Mack Publishing Company, Easton, Pa., 18th edition (1990).

For oral administration of the anti-cancer agent or treatment or the IGF-1R kinase inhibitor of Formula (I) that sensitizes tumor cells to the pro-apoptotic effects of the anti-cancer agent or treatment, tablets containing one or both of the active agents are combined with any of various excipients such as, for example, micro-crystalline cellulose, sodium citrate, calcium carbonate, dicalcium phosphate and glycine, along with various disintegrants such as starch (and preferably corn, potato or tapioca starch), alginic acid and certain complex silicates, together with granulation binders like polyvinyl pyrrolidone, sucrose, gelatin and acacia. Additionally, lubricating agents such as magnesium stearate, sodium lauryl sulfate and talc are often very useful for tableting purposes. Solid compositions of a similar type may also be employed as fillers in gelatin capsules; preferred materials in this connection also include lactose or milk sugar as well as high molecular weight polyethylene glycols. When aqueous suspensions and/or elixirs are desired for oral administration, active agents may be combined with various sweetening or flavoring agents, coloring matter or dyes, and, if so desired, emulsifying and/or suspending agents as well, together with such diluents as water, ethanol, propylene glycol, glycerin and various like combinations thereof.

For parenteral administration of either or both of the active agents, solutions in either sesame or peanut oil or in aqueous propylene glycol may be employed, as well as sterile aqueous solutions comprising the active agent or a corresponding water-soluble salt thereof. Such sterile aqueous solutions are preferably suitably buffered, and are also preferably rendered isotonic, e.g., with sufficient saline or glucose. These particular aqueous solutions are especially suitable for intravenous, intramuscular, subcutaneous and intraperitoneal injection purposes. The oily solutions are suitable for intra-articular, intramuscular and subcutaneous injection purposes. The preparation of all these solutions under sterile conditions is readily accomplished by standard pharmaceutical techniques well known to those skilled in the art.

Additionally, it is possible to topically administer either or both of the active agents, by way of, for example, creams, lotions, jellies, gels, pastes, ointments, salves and the like, in accordance with standard pharmaceutical practice. For example, a topical formulation comprising either the anti-cancer agent or treatment and/or an IGF-1R kinase inhibitor of Formula (I) that sensitizes tumor cells to the pro-apoptotic effects of the anti-cancer agent or treatment in about 0.1% (w/v) to about 5% (w/v) concentration can be prepared.

For veterinary purposes, the active agents can be administered separately or together to animals using any of the forms and by any of the routes described above. In a preferred embodiment, the anti-cancer agent or treatment and/or an IGF-1R kinase inhibitor of Formula (I) that sensitizes tumor cells to the pro-apoptotic effects of the anti-cancer agent or treatment are administered in the form of a capsule, bolus, tablet, liquid drench, by injection or as an implant. As an alternative, the active agents can be administered with the animal feedstuff, and for this purpose a concentrated feed additive or premix may be prepared for a normal animal feed. Such formulations are prepared in a conventional manner in accordance with standard veterinary practice.

The present invention also encompasses the use of a combination of a therapeutically effective amount of a combination of a anti-cancer agent or treatment that elevates pAkt levels in tumor cells and an IGF-1R kinase inhibitor of Formula (I), for the manufacture of a medicament for the treatment of tumors or tumor metastases in a patient in need thereof, wherein each inhibitor in the combination can be administered to the patient either simultaneously or sequentially. The present invention also encompasses the use of a synergistically effective combination of an anti-cancer agent or treatment that elevates pAkt levels in tumor cells and an IGF-1R kinase inhibitor of Formula (I), for the manufacture of a medicament for the treatment of tumors or tumor metastases in a patient in need thereof, wherein each inhibitor in the combination can be administered to the patient either simultaneously or sequentially. The present invention also encompasses the use of a combination of an anti-cancer agent or treatment that elevates pAkt levels in tumor cells and an IGF-1R kinase inhibitor of Formula (I), for the manufacture of a medicament for the treatment of abnormal cell growth in a patient in need thereof, wherein each inhibitor in the combination can be administered to the patient either simultaneously or sequentially. In an alternative embodiment of any of the above uses the present invention also encompasses the use of a combination of an anti-cancer agent or treatment that elevates pAkt levels in tumor cells and an IGF-1R kinase inhibitor of Formula (I) in combination with another anti-cancer agent or agent that enhances the effect of such an agent for the manufacture of a medicament for the treatment of tumors or tumor metastases in a patient in need thereof, wherein each inhibitor or agent in the combination can be administered to the patient either simultaneously or sequentially. In this context, the other anti-cancer agent or agent that enhances the effect of such an agent can be any of the agents listed herein above that can be added to the anti-cancer agent/treatment and IGF-1R kinase inhibitor of Formula (I) combination when treating patients.

The present invention further provides for any of the “methods of treatment” (or methods for reducing the side effects caused by treatment) described herein, a corresponding “method for manufacturing a medicament”, for administration with an anti-cancer agent or treatment that elevates pAkt levels in tumor cells and use with the same indications and under identical conditions or modalities described for the method of treatment, characterized in that an IGF-1R kinase inhibitor of Formula (I) is used, and such that where any additional agents, inhibitors or conditions are specified in alternative embodiments of the method of treatment they are also included in the corresponding alternative embodiment for the method for manufacturing a medicament. In an alternative embodiment, the present invention further provides for any of the “methods of treatment” (or methods for reducing the side effects caused by treatment) described herein, a corresponding “method for manufacturing a medicament” for use with the same indications and under identical conditions or modalities described for the method of treatment, characterized in that a combination of an anti-cancer agent or treatment that elevates pAkt levels in tumor cells and an IGF-1R kinase inhibitor of Formula (I) is used, such that where any additional agents, inhibitors or conditions are specified in alternative embodiments of the method of treatment they are also included in the corresponding alternative embodiment for the method for manufacturing a medicament.

The present invention further provides, for any of the methods, compositions or kits of the invention described herein in which a step or ingredient includes the phrase “comprising . . . a combination of an anti-cancer agents or treatments that elevates pAkt levels in tumor cells and an IGF-1R kinase inhibitor of Formula (I)”, a corresponding method, composition or kit in which that phrase is substituted with the phrase “consisting essentially of . . . a combination of an anti-cancer agents or treatments that elevates pAkt levels in tumor cells and an IGF-1R kinase inhibitor of Formula (I)”.

The present invention further provides, for any of the methods, compositions or kits of the invention described herein in which a step or ingredient includes the phrase “comprising . . . a combination of an anti-cancer agents or treatments that elevates pAkt levels in tumor cells and an IGF-1R kinase inhibitor of Formula (I)”, a corresponding method, composition or kit in which that phrase is substituted with the phrase “consisting of a combination of an anti-cancer agents or treatments that elevates pAkt levels in tumor cells and an IGF-1R kinase inhibitor of Formula (I)”.

The invention also encompasses a pharmaceutical composition that is comprised of a combination of an anti-cancer agent or treatment that elevates pAkt levels in tumor cells and an IGF-1R kinase inhibitor of Formula (I) in combination with a pharmaceutically acceptable carrier.

Preferably the composition is comprised of a pharmaceutically acceptable carrier and a non-toxic therapeutically effective amount of a combination of an anti-cancer agent or treatment that elevates pAkt levels in tumor cells and an IGF-1R kinase inhibitor of Formula (I) (including pharmaceutically acceptable salts of each component thereof).

Moreover, within this preferred embodiment, the invention encompasses a pharmaceutical composition for the treatment of disease, the use of which results in the inhibition of growth of neoplastic cells, benign or malignant tumors, or metastases, comprising a pharmaceutically acceptable carrier and a non-toxic therapeutically effective amount of a combination of an anti-cancer agent or treatment that elevates pAkt levels in tumor cells and an IGF-1R kinase inhibitor of Formula (I) (including pharmaceutically acceptable salts of each component thereof).

The term “pharmaceutically acceptable salts” refers to salts prepared from pharmaceutically acceptable non-toxic bases or acids. When a compound of the present invention is acidic, its corresponding salt can be conveniently prepared from pharmaceutically acceptable non-toxic bases, including inorganic bases and organic bases. Salts derived from such inorganic bases include aluminum, ammonium, calcium, copper (cupric and cuprous), ferric, ferrous, lithium, magnesium, manganese (manganic and manganous), potassium, sodium, zinc and the like salts. Particularly preferred are the ammonium, calcium, magnesium, potassium and sodium salts. Salts derived from pharmaceutically acceptable organic non-toxic bases include salts of primary, secondary, and tertiary amines, as well as cyclic amines and substituted amines such as naturally occurring and synthesized substituted amines. Other pharmaceutically acceptable organic non-toxic bases from which salts can be formed include ion exchange resins such as, for example, arginine, betaine, caffeine, choline, N′,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylameine, trimethylamine, tripropylamine, tromethamine and the like.

When a compound of the present invention is basic, its corresponding salt can be conveniently prepared from pharmaceutically acceptable non-toxic acids, including inorganic and organic acids. Such acids include, for example, acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like. Particularly preferred are citric, hydrobromic, hydrochloric, maleic, phosphoric, sulfuric and tartaric acids.

The pharmaceutical compositions of the present invention comprise a combination of an anti-cancer agent or treatment that elevates pAkt levels in tumor cells and an IGF-1R kinase inhibitor of Formula (I) (including pharmaceutically acceptable salts of each component thereof) as active ingredients, a pharmaceutically acceptable carrier and optionally other therapeutic ingredients or adjuvants. Other therapeutic agents may include those cytotoxic, chemotherapeutic or anti-cancer agents, or agents which enhance the effects of such agents, as listed above. The compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions may be conveniently presented in unit dosage form and prepared by any of the methods well known in the art of pharmacy.

In practice, the compounds represented by the combination of an anti-cancer agent or treatment that elevates pAkt levels in tumor cells and an IGF-1R kinase inhibitor of Formula (I) (including pharmaceutically acceptable salts of each component thereof) of this invention can be combined as the active ingredient in intimate admixture with a pharmaceutical carrier according to conventional pharmaceutical compounding techniques. The carrier may take a wide variety of forms depending on the form of preparation desired for administration, e.g. oral or parenteral (including intravenous). Thus, the pharmaceutical compositions of the present invention can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion, or as a water-in-oil liquid emulsion. In addition to the common dosage forms set out above, a combination of an anti-cancer agent or treatment that elevates pAkt levels in tumor cells and an IGF-1R kinase inhibitor of Formula (I) (including pharmaceutically acceptable salts of each component thereof) may also be administered by controlled release means and/or delivery devices. The combination compositions may be prepared by any of the methods of pharmacy. In general, such methods include a step of bringing into association the active ingredients with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

Thus, the pharmaceutical compositions of this invention may include a pharmaceutically acceptable carrier and a combination of an anti-cancer agent or treatment that elevates pAkt levels in tumor cells and an IGF-1R kinase inhibitor of Formula (I) (including pharmaceutically acceptable salts of each component thereof). A combination of an anti-cancer agent or treatment that elevates pAkt levels in tumor cells and an IGF-1R kinase inhibitor of Formula (I) (including pharmaceutically acceptable salts of each component thereof), can also be included in pharmaceutical compositions in combination with one or more other therapeutically active compounds. Other therapeutically active compounds may include those cytotoxic, chemotherapeutic or anti-cancer agents, or agents which enhance the effects of such agents, as listed above.

Thus in one embodiment of this invention, a pharmaceutical composition can comprise a combination of an anti-cancer agent or treatment that elevates pAkt levels in tumor cells and an IGF-1R kinase inhibitor of Formula (I) in combination with another anticancer agent, wherein said anti-cancer agent is a member selected from the group consisting of alkylating drugs, antimetabolites, microtubule inhibitors, podophyllotoxins, antibiotics, nitrosoureas, hormone therapies, kinase inhibitors, activators of tumor cell apoptosis, and antiangiogenic agents.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In preparing the compositions for oral dosage form, any convenient pharmaceutical media may be employed. For example, water, glycols, oils, alcohols, flavoring agents, preservatives, coloring agents, and the like may be used to form oral liquid preparations such as suspensions, elixirs and solutions; while carriers such as starches, sugars, microcrystalline cellulose, diluents, granulating agents, lubricants, binders, disintegrating agents, and the like may be used to form oral solid preparations such as powders, capsules and tablets. Because of their ease of administration, tablets and capsules are the preferred oral dosage units whereby solid pharmaceutical carriers are employed. Optionally, tablets may be coated by standard aqueous or nonaqueous techniques.

A tablet containing the composition of this invention may be prepared by compression or molding, optionally with one or more accessory ingredients or adjuvants. Compressed tablets may be prepared by compressing, in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent. Each tablet preferably contains from about 0.05 mg to about 5 g of the active ingredient and each cachet or capsule preferably contains from about 0.05 mg to about 5 g of the active ingredient.

For example, a formulation intended for the oral administration to humans may contain from about 0.5 mg to about 5 g of active agent, compounded with an appropriate and convenient amount of carrier material that may vary from about 5 to about 95 percent of the total composition. Unit dosage forms will generally contain between from about 1 mg to about 2 g of the active ingredient, typically 25 mg, 50 mg, 100 mg, 200 mg, 300 mg, 400 mg, 500 mg, 600 mg, 800 mg, or 1000 mg.

Pharmaceutical compositions of the present invention suitable for parenteral administration may be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Pharmaceutical compositions of the present invention can be in a form suitable for topical sue such as, for example, an aerosol, cream, ointment, lotion, dusting powder, or the like. Further, the compositions can be in a form suitable for use in transdermal devices. These formulations may be prepared, utilizing a combination of a combination of an anti-cancer agent or treatment that elevates pAkt levels in tumor cells and an IGF-1R kinase inhibitor of Formula (I) (including pharmaceutically acceptable salts of each component thereof) of this invention, via conventional processing methods. As an example, a cream or ointment is prepared by admixing hydrophilic material and water, together with about 5 wt % to about 10 wt % of the compound, to produce a cream or ointment having a desired consistency.

Pharmaceutical compositions of this invention can be in a form suitable for rectal administration wherein the carrier is a solid. It is preferable that the mixture forms unit dose suppositories. Suitable carriers include cocoa butter and other materials commonly used in the art. The suppositories may be conveniently formed by first admixing the composition with the softened or melted carrier(s) followed by chilling and shaping in molds.

In addition to the aforementioned carrier ingredients, the pharmaceutical formulations described above may include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions containing a combination of an anti-cancer agent or treatment that elevates pAkt levels in tumor cells and an IGF-1R kinase inhibitor of Formula (I) (including pharmaceutically acceptable salts of each component thereof) may also be prepared in powder or liquid concentrate form.

Dosage levels for the compounds of the combination of this invention will be approximately as described herein, or as described in the art for these compounds. It is understood, however, that the specific dose level for any particular patient will depend upon a variety of factors including the age, body weight, general health, sex, diet, time of administration, route of administration, rate of excretion, drug combination and the severity of the particular disease undergoing therapy.

In further embodiments of any of the above methods, compositions or kits of this invention where an IGF-1R kinase inhibitor of Formula (I) is used, the IGF-1R kinase inhibitor comprises any compound of Formula (I) as described in US Published Patent Application US 2006/0235031 (e.g. OSI-906).

Data from the experiments described herein also indicate that IGF-1R or IR activity (or pIGF-1R or p-IR levels) may be useful biomarkers to identify those tumors that will likely receive maximal benefit from the combination of an anti-cancer agent or treatment that elevates pAkt levels in tumor cells (e,g. paclitaxel, doxorubicin) and an IGF-1R kinase inhibitor, such as for example a compound of Formula (I) (e.g. OSI-906). For tumor cells where the combination of an IGF-1R kinase inhibitor of Formula (I) (e.g. OSI-906) with said anti-cancer agent or treatment achieves a synergistic promotion of apoptosis and inhibition of cell survival, the effects may be dose dependent.

Accordingly, the present invention also provides a method of identifying tumor cells that will respond most favorably to treatment with a combination of an anti-cancer agent or treatment that elevates pAkt levels in tumor cells and an IGF-1R kinase inhibitor, comprising: contacting a sample of tumor cells with said anti-cancer agent or treatment that elevates pAkt levels in tumor cells, determining whether said anti-cancer agent or treatment stimulates phosphorylation of IGF-1R or IR in the tumor cells, by comparing the level of p-IGF-1R or p-IR in tumor cells contacted with said anti-cancer agent or treatment to the level of p-IGF-1R or p-IR in an identical sample of tumor cells either not contacted with said anti-cancer agent or treatment, or contacted with a lower concentration of said anti-cancer agent or treatment, and predicting whether the sample tumor cells will respond favorably to treatment with a combination of an anti-cancer agent or treatment that elevates pAkt levels in tumor cells and an IGF-1R kinase inhibitor, wherein the higher the level of p-IGF-1R or p-IR induced by said anti-cancer agent or treatment in tumor cells, the greater likelihood that the tumor cells will respond favorably to treatment with a combination of an anti-cancer agent or treatment that elevates pAkt levels in tumor cells and an IGF-1R kinase inhibitor.

The present invention also provides a method of identifying tumor cells that will respond most favorably to treatment with a combination of an anti-cancer agent or treatment that elevates pAkt levels in tumor cells and an IGF-1R kinase inhibitor, comprising contacting a sample of tumor-cells with said anti-cancer agent or treatment that elevates pAkt levels in tumor cells, determining whether said anti-cancer agent or treatment stimulates phosphorylation of IGF-1R or IR in the tumor cells, by comparing the level of p-IGF-1R or p-IR in tumor cells contacted with said anti-cancer agent or treatment to the level of p-IGF-1R or p-IR in an identical sample of tumor cells either not contacted with said anti-cancer agent or treatment, or contacted with a lower concentration (e.g. a non-efficacious dose or level) of said anti-cancer agent or treatment, comparing the level of p-IGF-1R or p-IR in the sample of tumor cells contacted with said anti-cancer agent or treatment with the level of p-IGF-1R or p-IR in a control sample of tumor cells contacted with said anti-cancer agent or treatment, wherein said control sample of tumor cells is known to respond favorably to treatment with said combination of an anti-cancer agent or treatment that elevates pAkt levels in tumor cells and an IGF-1R kinase inhibitor (e.g. H292 tumor cells treated with paclitaxel; A673 tumor cells treated with doxorubicin), and predicting whether the sample tumor cells will respond favorably to treatment with a combination of an anti-cancer agent or treatment that elevates pAkt levels in tumor cells and an IGF-1R kinase inhibitor, wherein the higher the level of p-IGF-1R or p-IR induced by said anti-cancer agent or treatment in tumor cells, the greater likelihood that the tumor cells will respond favorably to treatment with a combination of an anti-cancer agent or treatment that elevates pAkt levels in tumor cells and an IGF-1R kinase inhibitor. In this method, the step of comparing the level of p-IGF-1R or p-IR in the sample of tumor cells contacted with said anti-cancer agent or treatment with the level of p-IGF-1R or p-IR in a control sample of tumor cells contacted with said anti-cancer agent or treatment allows comparison to a tumor model that has a well characterized response, and where the levels of p-IGF-1R or p-IR predictive of that response are defined, and thus assists in predicting the type of response to be expected from the tumor cells that have not been previously treated.

In one embodiment of the above methods of identifying tumor cells that will respond most favorably to treatment with a combination of an anti-cancer agent or treatment that elevates pAkt levels in tumor cells and an IGF-1R kinase inhibitor, the IGF-1R kinase inhibitor comprises any “IGF-1R kinase inhibitors other than IGF-1R kinase inhibitors of Formula (I)”, as defined herein above, e.g. low molecular weight inhibitors, antibodies or antibody fragments, antisense constructs, small inhibitory RNAs, and ribozymes. In another embodiment of the above methods of identifying tumor cells that will respond most favorably to treatment with a combination of an anti-cancer agent or treatment that elevates pAkt levels in tumor cells and an IGF-1R kinase inhibitor, the IGF-1R kinase inhibitor comprises a compound of Formula (I), e.g. OSI-906. In another embodiment, the anti-cancer agent or treatment that elevates pAkt levels in tumor cells is a chemotherapeutic agent or a gene-targetted anti-cancer agent. In another embodiment, the anti-cancer agent or treatment that elevates pAkt levels in tumor cells is selected from anthracyclins, doxorubicin, daunorubicin, DNA-damaging agents, cisplatin, carboplatin, topoisomerase inhibitors, camptothecin, etoposide, microtubule-directed agents, vincristine, colchicines, vinblastine, decetaxel, paclitaxel, ionizing radiation, rapamycin, rapalogs, CCI-779, RAD001 trastuzumab, and A443654. In another embodiment, the sample of tumor cells is selected from Ewing's sarcoma, NSCL, pancreatic, head and neck, colon, ovarian or breast cancer cells. In another embodiment the sample of tumor cells is a tumor or tumor biopsy from a patient with cancer.

For assessment of tumor cell p-IGF-1R or p-IR levels in a tumor or tumor biopsy from a patient, standard methods known in the art may be used for obtaining patient samples. Treatment of the tumor cells with the anti-cancer agent or treatment that elevates pAkt levels in tumor cells can be done in vivo, followed by tumor biopsy to assay p-IGF-1R or p-IR; or ex vivo after sampling tumor cells from the tumor. After treatment in vivo, the biopsy sample can be subjected to a variety of well-known post-collection preparative and storage techniques (e.g., protein extraction, fixation, freezing, ultrafiltration, concentration, etc.) prior to assessing the amount of the phosphorylated protein in the sample.

This invention will be better understood from the Experimental Details that follow. However, one skilled in the art will readily appreciate that the specific methods and results discussed are merely illustrative of the invention as described more fully in the claims which follow thereafter, and are not to be considered in any way limited thereto.

### Experimental Details

It has not been previously determined if it was possible to combine an anti-cancer agent/treatment that elevates pAkt levels in tumor cells and an IGF-1R kinase inhibitor of Formula (I). Unlike cytotoxic chemotherapies that often share similar toxicities, molecularly-targeted agents (i.e. gene-targeted agents) tend to have different, non-overlapping toxicities and thus identifying cocktails or combinations of such targeted agents and other anti-cancer agent/treatments to block cancer cell growth may be more clinically feasible. Synergistic tumor cell growth-inhibiting behavior of some IGF-1R pathway inhibiting agents when combined with anti-cancer agents or treatments that elevate pAkt levels in tumor cells has been previously reported (e.g. Min, Y. et al. (2005) Gut 54:591-600; Goetsch, L. et al. (2005) Int. J. Cancer 113:316-328; US Published Patent Application No. 2004/0209930). Others have reported only additive effects when IGF-1R pathway inhibiting agents are combined with such anti-cancer agents or treatments (e.g. Hopfner, M. et al. (2006) Endocrine-Related Cancer 13:135-149; Baradari, V. et al. (2005) Z Gastroenterol. 43 DOI: 10.1055/s-2005-920141). In the experiments described herein Compound D, an IGF-1R kinase inhibitor of Formula (I), was found to consistently produce synergistic effects when combined with a anti-cancer agent that elevates pAkt levels in tumor cells.

Thus, herein it is demonstrated that an IGF-1R kinase inhibitor of Formula (I) can sensitize tumor cell lines to the pro-apoptotic effects of anti-cancer agents/treatments that elevate pAkt levels in tumor cells. Thus combining an anti-cancer agent/treatment that elevates pAkt levels in tumor cells and an IGF-1R kinase inhibitor of Formula (I) should be useful clinically in treating patients with cancer, such as breast cancer for example.

### Materials and Methods

Drugs: IGF-1R kinase inhibitors useful in this invention include compounds represented by Formula (I) (see above), as described in US Published Patent Application US 2006/0235031, where their preparation is described in detail. Compound D represents an IGF-1R kinase inhibitor according to Formula (I), also referred to in some places herein as OSI-906 (cis-3-[8-amino-1-(2-phenyl-quinolin-7-yl)-imidazo[1,5-a]pyrazin-3-yl]-1-methyl-cyclobutanol). It has the structure as follows:

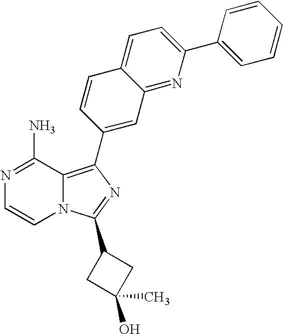

Anti-IGF-1R neuralizing antibodies included αIR3 (Calbiochem (EMD), La Jolla, Calif.) and MAB391 (R&D systems, Minneapolis, Minn.). Other compounds or drugs were obtained from commercial sources.

Cell lines: The human breast cancer cell line MDA-MB-231, Ewing's sarcoma cell lines A673 and SK-ES-3, NSCL cancer cell lines H460, H292, H322, H358 and Calu6, and SCCHN (squamous cell carcinoma of the head and neck) cell line MDA-1186 were purchased from the American Type Culture Collection (ATCC). They were grown in media as prescribed by the ATCC, containing 10% FCS.

Measurement of Cell Proliferation: Cell proliferation was determined using the Cell Titer Glo assay (Promega Corporation, Madison, Wis.). Tumor cells were seeded at a density of 3000 cells per well in a 96-well plate. 24 hours after plating cells were dosed with varying concentrations of drug, either as a single agent or in combination. Using parallel replicate plates, the signal for Cell Titer Glo was determined 24 hours after dosing.

Measurement of apoptosis: Induction of apoptosis as measured by increased Caspase 3/7 activity was determined using the Caspase 3/7 Glo assay (Promega Corporation, Madison, Wis.). Cell lines were seeded at a density of 3000 cells per well in a 96-well plate. 24 hours after plating cells were dosed with varying concentrations of drug, either as a single agent or in combination. The signal for Caspase 3/7 Glo was determined 24 hours after dosing. The caspase 3/7 activity was normalized to cell number per well, using a parallel plate treated with Cell Titer Glo (Promega Corporation, Madison, Wis.). Signal for each well was normalized using the following formula: Caspase 3/7 Glo luminescence units/Cell Titer Glo fraction of DMSO control. All graphs were generated using PRISM® software (Graphpad Software, San Diego, Calif.).

Preparation of Protein Lysates and Western Blotting:

Cell extracts were prepared by detergent lysis (50 mM Tris-HCl, pH 8.0, 150 mM NaCl, 1% NP-40, 0.5% sodium deoxycholate, 0.1% SDS, containing protease inhibitor (P8340, Sigma, St. Louis, Mo.) and phosphatase inhibitor (P5726, Sigma, St. Louis, Mo.) cocktails. The soluble protein concentration was determined by micro-BSA assay (Pierce, Rockford Ill.). Protein immunodetection was performed by electrophoretic transfer of SDS-PAGE separated proteins to nitrocellulose, incubation with antibody, and chemiluminescent second step detection (PicoWest; Pierce, Rockford, Ill.). The antibodies included: phospho-Akt(473) and total Akt. Both antibodies were obtained from Cell Signaling Technology, Inc. (Danvers, Mass.). For analysis of an agent's effect on the phosphorylation of downstream signaling proteins, cell lines were grown to approximately 70% confluency, at which time the indicated agent was added at the indicated concentration, and cells were incubated at 37° C. for 24 hours. The media was removed, cells were washed two times with PBS, and cells were lysed as previously described.

Analysis of RTKs Via a Proteome Array:

Proteome profiler arrays housing 42 different RTKs were purchased from R&D systems (Minneapolis, Minn.) and processed according to the manufacturer's protocol. RTKs included on the array include: HER1, HER2, HER3, HER4, FGFR1, FGFR2a, FGFR3, FGFR4, IR, IGF-1R, Ax1, Dtk, Mer, HGFR, MSPR, PDGFRα, PDGFRβ, SCFR, Flt-3, M-CSFR, c-Ret, ROR1, ROR2, Tie-1, Tie-2, TrkA, TrkB, TrkC, VEGFR1, VEGFR2, VEGFR3, MuSK, EphA1, EphA2, EphA3, EphA4, EphA6, EphA7, EphB1, EphB2, EphB4, EphB6. This array was used as an RTK capture assay for determining pIGF-1R levels.

Animals

Female athymic nude nu/nu CD-1 mice (6-8 wks, 22-29 g) were obtained from Charles River Laboratories (Wilmington, Mass.). Animals were allowed to acclimate for a minimum of one week prior to initiation of a study. Throughout the studies, animals were allowed sterile rodent chow and water ad libitum, and animals were maintained under specific pathogen free conditions. All animal studies were conducted at OSI facilities with the approval of the Institutional Animal Care and Use Committee in an American Association for Accreditation of Laboratory Animal Care (AAALAC)-accredited vivarium and in accordance with the Institute of Laboratory Animal Research (Guide for the Care and Use of Laboratory Animals, NIH, Bethesda, Md.).

In Vivo Pharmacodynamic Efficacy Study:

NCI—H292 NSCLC tumor cells were harvested from cell culture flasks during exponential cell growth, washed twice with sterile PBS, counted and resuspended in PBS to a suitable concentration before s.c. implantation on the right flank of female nu/nu CD-1 mice. Tumors were established to 200+/−50 mm in size before randomization into treatment groups. Paclitaxel or cisplatin were administered at the indicated dose, and tumors from treated mice were harvested at 24 hours after dosing. Tumors were immediately frozen in liquid nitrogen, and subsequently prepared for phosphor-proteomic analysis.

### Results/Discussion

Studies on the Effect of a Combination of an Anti-Cancer Agent that Elevates pAkt Levels and an IGF-1R Kinase Inhibitor of Formula (I) on Tumor Cells.

Breast Tumor Cell Lines:

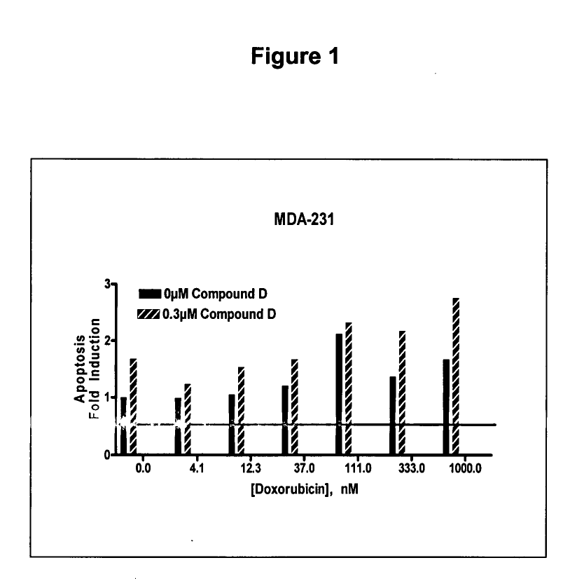

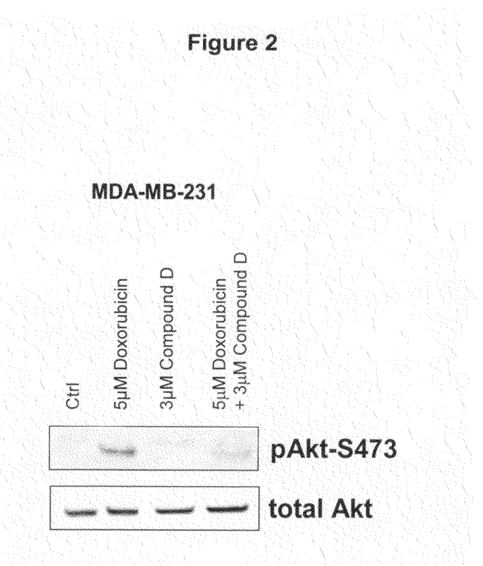

For the breast tumor cell line MDA-MB-231, treatment with doxorubicin for 24 hours promotes an increase in Akt phosphorylation (pAkt-S473) (FIG. 2). When cells are treated with the combination of doxorubicin and Compound D, it is found that Compound D is efficacious at inhibiting the increase in Akt phosphorylation provoked by doxorubicin. These effects translate into enhanced induction in apoptosis. For cells treated for 24 hours with doxorubicin alone, only a small induction in apoptosis is observed (FIG. 1). However, when MDA-MB-231 cells are co-treated with the combination of 1 μM doxorubicin and 0.3 μM compound D (FIG. 1), an induction in apoptosis of about 3-fold is evoked.

The ability of a cytotoxic anti-cancer agent to evoke an increase in Akt phosphorylation has been previously described, and this has been postulated to limit such an agent's efficacy toward inhibiting cell proliferation and survival as a single agent. It was found that compound D effectively inhibit the increase in Akt phosphorylation promoted by the anti-cancer agent doxorubicin. This data suggest that compound D might cooperate with select anti-cancer agents such as doxorubicin to potentiate apoptosis. Indeed, it was found that at 24 hours after dosing, compound D considerably potentiates apoptosis in tumor cells treated with doxorubicin. This data suggests that for anti-cancer agents or treatments that exhibit the capacity to promote Akt phosphorylation, combination with an IGF-1R kinase inhibitor of Formula (I), such as Compound D, will likely augment the activities of such agents in patient tumors (e.g: to promote tumor apoptosis and growth inhibition).

Ewing's Sarcoma Tumor Cell Lines:

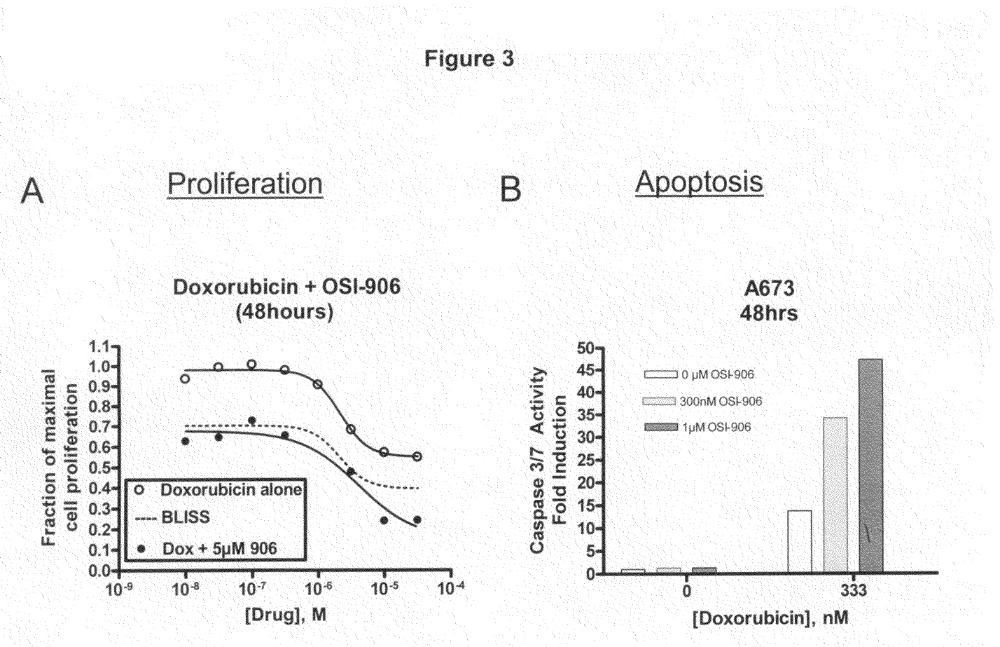

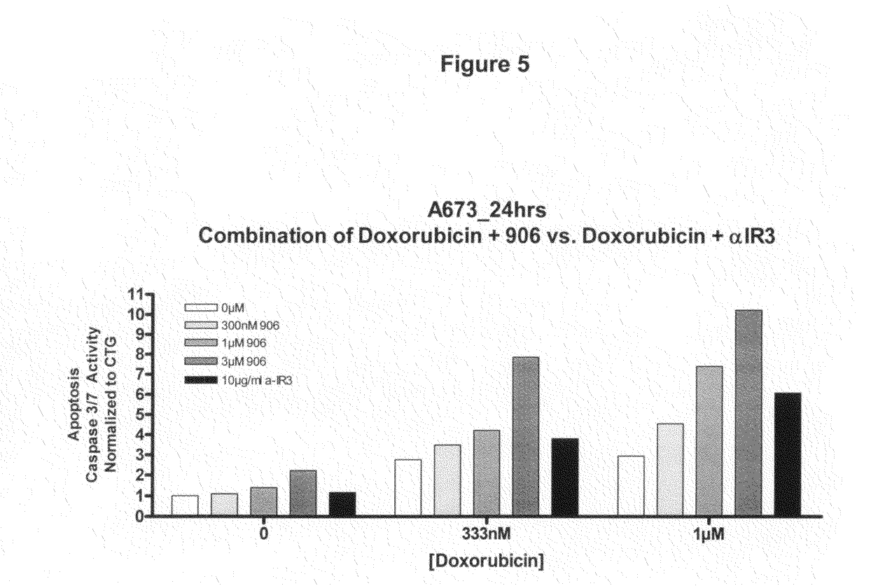

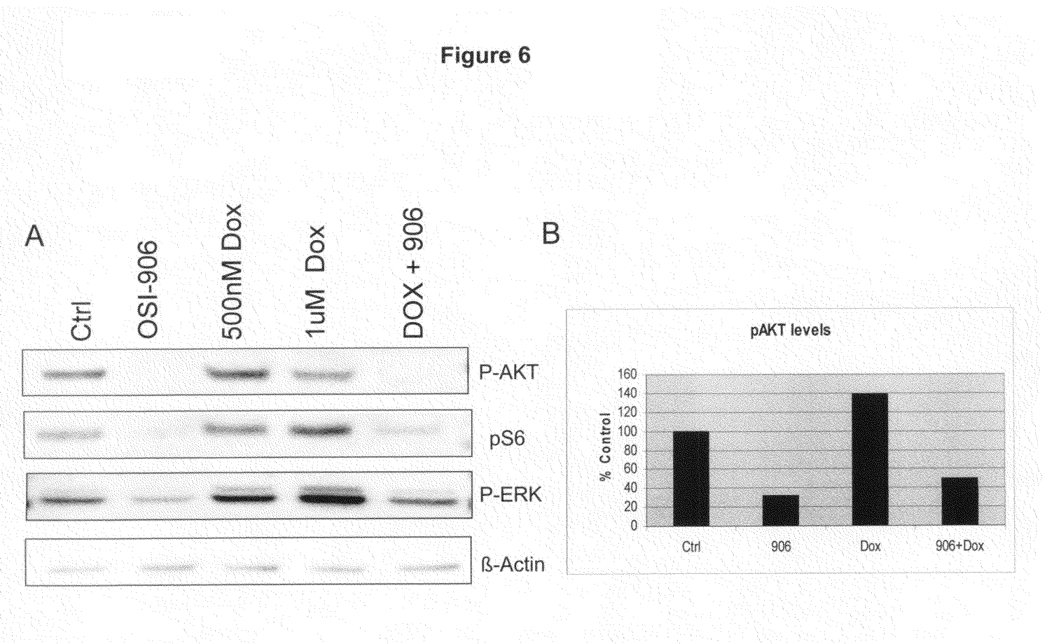

OSI-906 can demonstrate cooperative activity with the cytotoxic agent doxorubicin in ES tumor cell lines. For the Ewing's Sarcoma tumor cell lines A673 and SK-ES-1 the combination of OSI-906 and doxorubicin yields a synergistic promotion of apoptosis and inhibition of overall cell growth, FIG. 2-3. For analysis of overall cell growth, FIG. 3A, the experimental data for the combination was compared with the theoretical expectation for additivity as calculated using the Bliss additivism model, and is denoted by the dotted line in FIG. 1A. For A673 tumor cells the combination of doxorubicin with either OSI-906 or the neutralizing IGF-1R antibody, a-IR3, yields a synergistic increase in doxorubicin-mediated apoptosis. However, the extent of cooperativity appears to be greater at higher concentrations of OSI-906, i.e. 3 μM, than that achieved maximally by a-IR3, FIG. 5. For A673 tumor cells, the activities for both the MAPK and Akt pathway appear to be mediated by IGF-1R, and OSI-906 achieves inhibition of both pathways. Treatment with doxorubicin yields an increase in the activation states for both Akt and Erk, and this gain in activity can be inhibited upon co-treatment with OSI-906, FIG. 6.

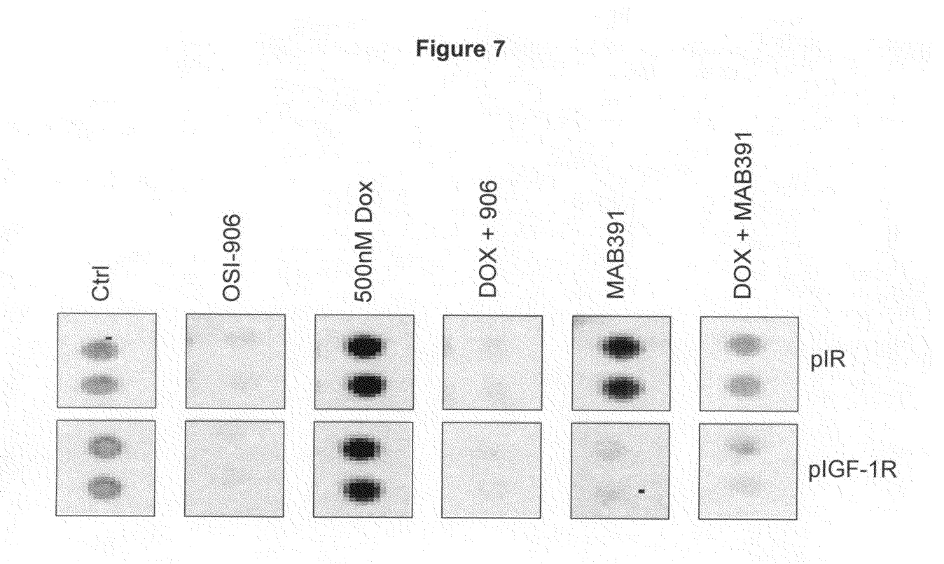

Mechanistically, the observed cooperativity between OSI-906 and doxorubicin appears to be due to the capacity of doxorubicin to drive an increase in the activation states for both IR and IGF-1R. OSI-906 (3 μM) achieves inhibition for both pIR and pIGF-1R in A673 ES tumor cells, FIG. 7. The observed inhibition of IR could be due to blockade of heterodimers with IGF-1R or direct inhibition of IR. Treatment with doxorubicin promotes an upregulation of the phosphorylation state for both IR and IGF-1R, and this is blocked upon co-treatment with OSI-906. Treatment of A673 ES tumor cells with MAB391, a neutralizing antibody directed against IGF-1R, achieves inhibition of IGF-1R phosphorylation that is comparable to that seen for the IGF-1R TKI inhibitor OSI-906. However, this is accompanied by an increase in the phosphorylation state for IR, suggesting that IR activity may compensate for IGF-1R under conditions where IGF-1R is specifically inhibited. Although MAB-391, can fully inhibit doxorubicin activation of IGF-1R, it only partially blocks doxorubicin activation of IR. These data suggest that TKI and antibody inhibitors of IGF-1R may exhibit differential activities, alone or when combined with cytotoxic agents. These data also indicate that monitoring for the phosphorylation of IR and/or IGF-1R may be a useful marker to identify those tumors likely to receive the most benefit from this specific combination of agents. Measurements for pIR and/or pIGF-1R could be made either for tissues obtained directly from the tumor or for tumor cells in circulation.

NSCLC Tumor Cell Lines:

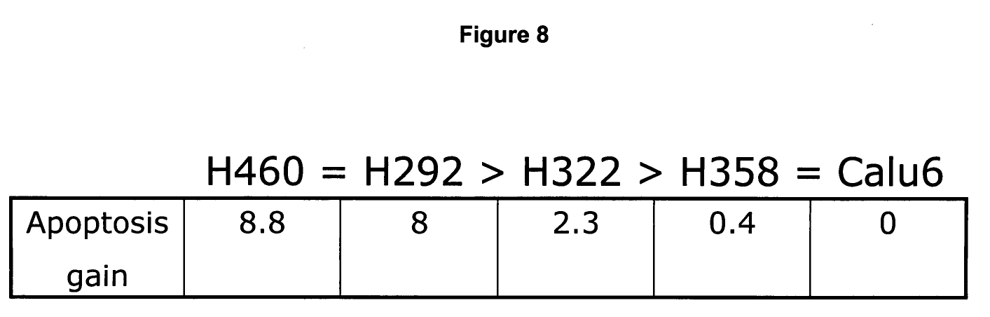

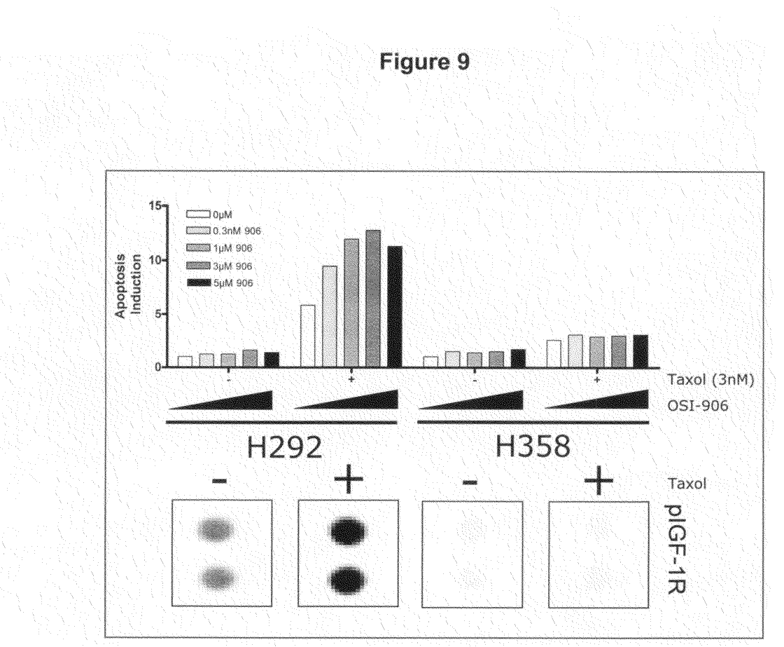

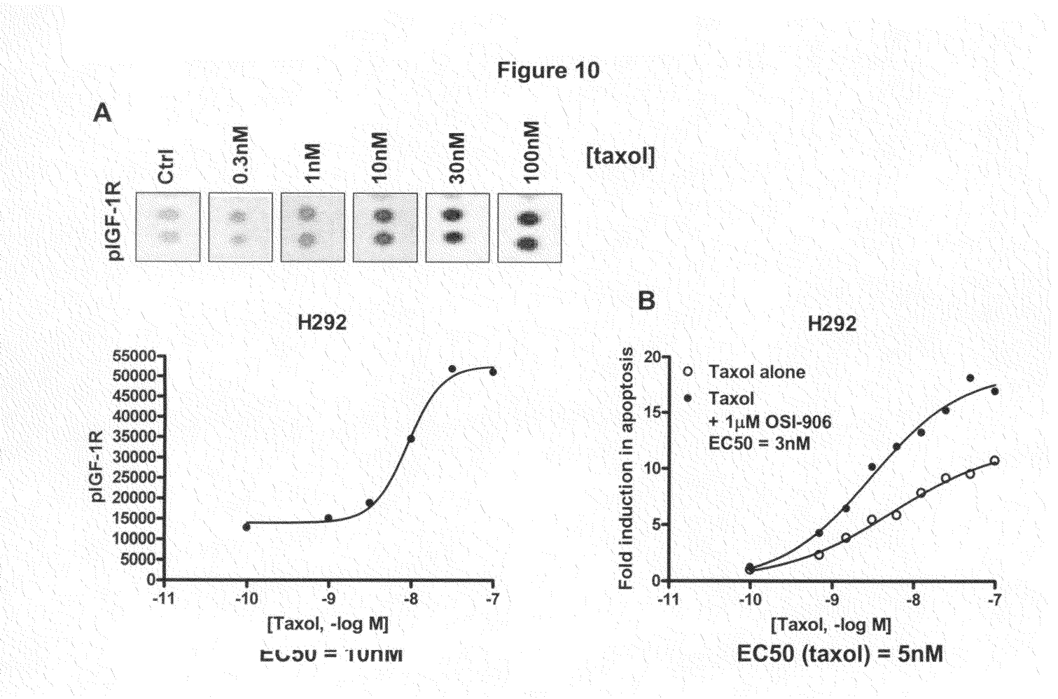

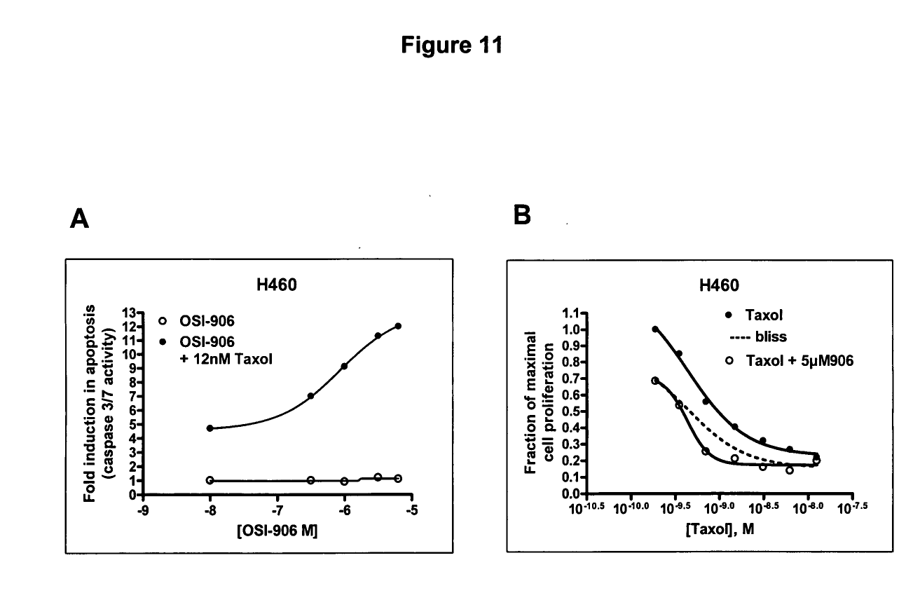

OSI-906 can cooperate with taxol (paclitaxel) in NSCLC tumor cell lines to promote a synergistic induction in apoptosis and block overall cell growth. In an analysis of 5 NSCLC tumor cell lines (H460, H292, H322, H358, and Calu6), OSI-906 was found to be synergistic with taxol, in terms of promoting apoptosis, for 3/5 of these tumor cell lines, FIG. 8. The apoptosis gain is defined as the fold gain in apoptosis, as measured by caspase 3/7 activity, above that achieved by taxol alone. An apoptosis gain of >2 was defined as synergistic. Herein, a strong synergistic gain in apoptosis (>5) was observed for H460 and H292 tumor cells, FIG. 7. For NSCLC, the capacity of taxol to promote an increase in the phosphorylation state of IGF-1R correlates with synergy when administered in combination with OSI-906, FIG. 9. Herein, synergy for the combination of OSI-906 and taxol was observed for H292, but not H358, tumor cells. Only in H292 tumor cells did taxol promote an increase in IGF-1R phosphorylation. The ability of taxol to promote phosphorylation of IGF-1R is dose dependent, EC50=10 nM, and closely correlates with the apoptosis synergy for OSI-906 and taxol, FIG. 10. These data indicate that pIGF-1R activity may be a useful biomarker to identify those NSCLC tumors that will likely receive maximal benefit from the combination of a taxol containing regimen and OSI-906. For tumor cell lines including H460 where the combination of OSI-906 with taxol achieves a synergistic promotion of apoptosis and inhibition of cell survival, the effects are dose dependent. As a single agent OSI-906 fails to promote apoptosis or significantly inhibit cell growth, however it does increase the pro-apoptotic effects of doxorubicin and achieves greater than additive inhibition of overall cell growth when administered with taxol, FIG. 11.

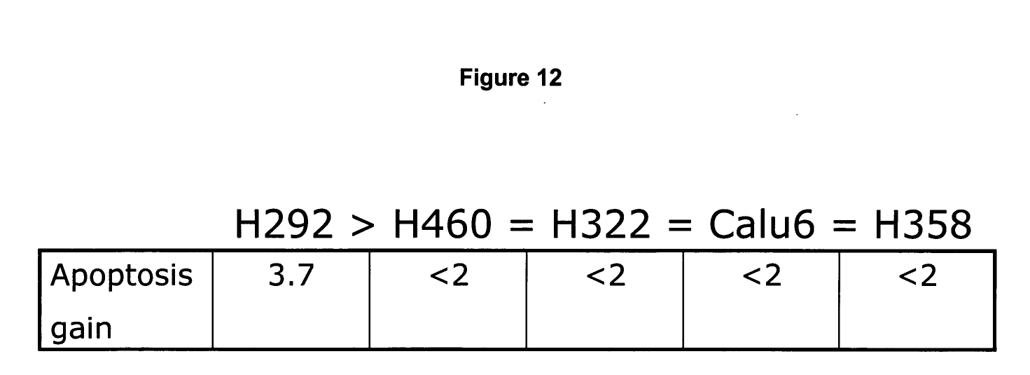

For select NSCLC tumor cell lines, OSI-906 can synergize with cisplatin to promote a synergistic gain in apoptosis, FIG. 12. For H292 tumor cells, the combination of OSI-906 and cisplatin achieved greater than a 3-fold increase in apoptosis compared with the activity for the single agents.

SCCHN Tumor Cell Lines:

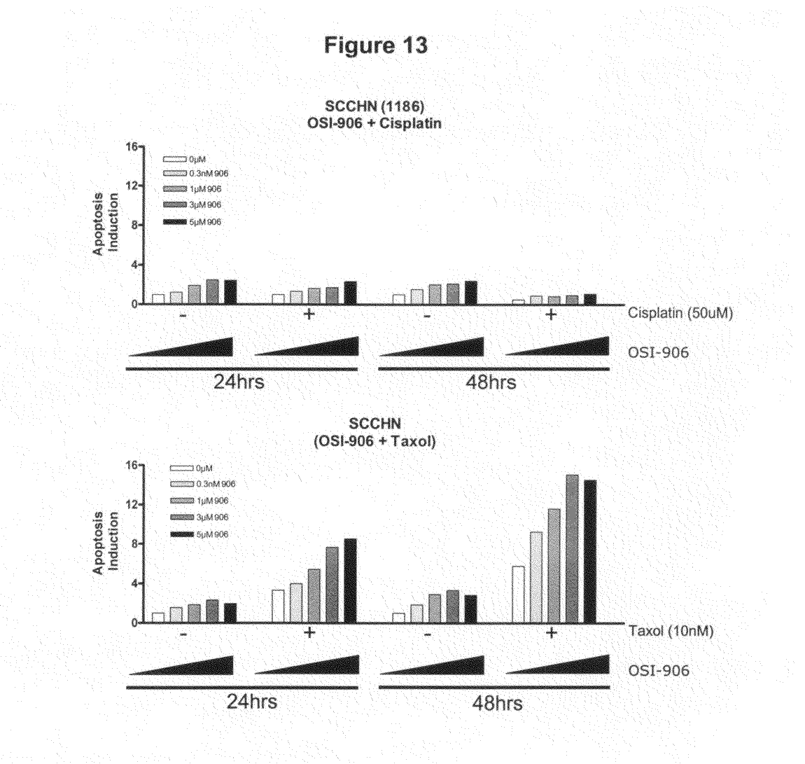

The ability of OSI-906 to synergize with taxol extends to cell lines derived from head and neck tumors. For MDA-1186 tumor cells, the combination of OSI-906 with taxol promoted a dose dependent increase in apoptosis, even though OSI-906 did not achieve significant apoptosis as a single agent, FIG. 13. The ability of OSI-906 and taxol to synergize in MDA-1186 tumor cells correlated with the ability of taxol to promote phosphorylation of the IGF-1R complex, data not shown.

Sequential Treatment

We find that the basal levels for phosphorylation of IGF-1R are modest for H292 cells both in vitro and in vivo. Paclitaxel treatment evokes a time-dependent increase in pIGF-1R in vitro. A modest increase is observed at 2 hours after dosing, however this is further increased by prolonged exposure (i.e. 6 hours) (FIGS. 1A, 2). A similar trend is observed for pAkt, where 2 hour treatment with paclitaxel evokes a modest increase in the Akt survival pathway, however this pathway is further driven upon prolonged 6 hour exposure to paclitaxel, and is sustained for at least 24 hours (FIG. 2). In vivo we find that the low basal levels for pIGF-1R are upregulated following exposure to paclitaxel for 24 hours, FIG. 1B. Since prolonged exposure to paclitaxel (i.e. hours) is necessary to fully activate the IGF-1R survival pathway, these data indicate that treatment with a chemotherapeutic agent that induces phosphorylation of Akt, such as paclitaxel, prior to treatment with an IGF-1R inhibitor such as OSI-906, will produce superior efficacy to treatment by simultaneous administration of these two agents.

Similar data indicating that superior efficacy will be achieved by administration of a chemotherapeutic agent that induces phosphorylation of Akt prior to treatment with an IGF-1R inhibitor such as OSI-906, was obtained for the human head and neck tumor cell line 1186, using paclitaxel and OSI-906; for the human NSCL tumor cell line H322, using paclitaxel and OSI-906; and for the human Ewing's sarcoma tumor cell line A673, using doxorubicin and OSI-906. In all of these cases a synergistic induction of apoptosis was observed in response to the combination. These data further suggest that pretreatment with a chemotherapeutic agent that induces phosphorylation of Akt prior to treatment with an IGF-1R inhibitor such as OSI-906 will be more effective than simultaneous administration of two such agents, and that this sequential regimen will likely be effective with any agent that induces pAKT, and in multiple tumor types.

### Abbreviations

EGF, epidermal growth factor; EGFR, epidermal growth factor receptor; EMT, epithelial-to-mesenchymal transition; MET, mesenchymal-to-epithelial transition; NSCL, non-small cell lung; NSCLC, non-small cell lung cancer; HNSCC, head and neck squamous cell carcinoma; CRC, colorectal cancer; MBC, metastatic breast cancer; Brk, Breast tumor kinase (also known as protein tyrosine kinase 6 (PTK6)); FCS, fetal calf serum; LC, liquid chromatography; MS, mass spectrometry; IGF-1, insulin-like growth factor-1; IR, insulin receptor; TGFα, transforming growth factor alpha; HB-EGF, heparin-binding epidermal growth factor; LPA, lysophosphatidic acid; IC₅₀, half maximal inhibitory concentration; pY, phosphotyrosine; wt, wild-type; PI3K, phosphatidyl inositol-3 kinase; GAPDH, glyceraldehyde 3-phosphate dehydrogenase; MAPK, mitogen-activated protein kinase; PDK-1,3-Phosphoinositide-Dependent Protein Kinase 1; Akt, also known as protein kinase B, is the cellular homologue of the viral oncogene v-Akt; pAkt, phosphorylated Akt; mTOR, mammalian target of rapamycin; 4EBP 1, eukaryotic translation initiation factor-4E (mRNA cap-binding protein) Binding Protein-1, also known as PHAS-I; p70S6K, 70 kDa ribosomal protein-S6 kinase; eIF4E, eukaryotic translation initiation factor-4E (mRNA cap-binding protein); Raf, protein kinase product of Raf oncogene; MEK, ERK kinase, also known as mitogen-activated protein kinase kinase; ERK, Extracellular signal-regulated protein kinase, also known as mitogen-activated protein kinase; PTEN, “Phosphatase and Tensin homologue deleted on chromosome 10”, a phosphatidylinositol phosphate phosphatase; pPROTEIN, phospho-PROTEIN, “PROTEIN” can be any protein that can be phosphorylated, e.g. EGFR, Akt, IGF-1R, IR, ERK, S6 etc; PBS, Phosphate-buffered saline; RTK, Receptor Tyrosine Kinase; TGI, tumor growth inhibition; WFI, Water for Injection; SDS, sodium dodecyl sulfate; ErbB2, “v-erb-b2 erythroblastic leukemia viral oncogene homolog 2”, also known as HER-2; ErbB3, “v-erb-b2 erythroblastic leukemia viral oncogene homolog 3”, also known as HER-3; ErbB4, “v-erb-b2 erythroblastic leukemia viral oncogene homolog 4”, also known as HER-4; FGFR, Fibroblast Growth Factor Receptor; DMSO, dimethyl sulfoxide; “Taxol”, paclitaxel.

### INCORPORATION BY REFERENCE

All patents, published patent applications and other references disclosed herein are hereby expressly incorporated herein by reference.

### EQUIVALENTS

Those skilled in the art will recognize, or be able to ascertain, using no more than routine experimentation, many equivalents to specific embodiments of the invention described specifically herein. Such equivalents are intended to be encompassed in the scope of the following claims.

What is claimed is:

1. A method for treating tumors or tumor metastases in a patient, comprising administering to said patient simultaneously or sequentially a therapeutically effective amount of a combination of an anti-cancer agent or treatment that elevates pAkt levels in tumor cells and an IGF-1R kinase inhibitor of Formula (I).

2. The method of claim 1, wherein the patient is a human that is being treated for cancer.

3. The method of claim 1, wherein the anti-cancer agent or treatment and IGF-1R kinase inhibitor are co-administered to the patient in the same formulation.

4. The method of claim 1, wherein the anti-cancer agent or treatment and IGF-1R kinase inhibitor are co-administered to the patient in different formulations.

5. The method of claim 1, wherein the anti-cancer agent or treatment and IGF-1R kinase inhibitor are co-administered to the patient by the same route.

6. The method of claim 1, wherein the anti-cancer agent or treatment and IGF-1R kinase inhibitor are co-administered to the patient by different routes.

7. The method of claim 1, wherein the anti-cancer agent or treatment is selected from anthracyclins, doxorubicin, daunorubicin, DNA-damaging agents, cisplatin, carboplatin, topoisomerase inhibitors, camptothecin, etoposide, microtubule-directed agents, vincristine, colchicines, vinblastine, decetaxel, paclitaxel, ionizing radiation, rapamycin, rapalogs, CCI-779, RAD001, trastuzumab, and A443654.

8. The method of claim 1, additionally comprising administering to said patient one or more other anti-cancer agents.

9. The method of claim 1, wherein the administering to the patient is simultaneous.

10. The method of claim 1, wherein the administering to the patient is sequential.

11. A method for the treatment of cancer, comprising administering to a subject in need of such treatment an amount of an anti-cancer agent or treatment that elevates pAkt levels in tumor cells; and an amount of an IGF-1R kinase inhibitor of Formula (I); wherein at least one of the amounts is administered as a sub-therapeutic amount.

12. The method of claim 11, wherein the anti-cancer agent or treatment is selected from anthracyclins, doxorubicin, daunorubicin, DNA-damaging agents, cisplatin, carboplatin, topoisomerase inhibitors, camptothecin, etoposide, microtubule-directed agents, vincristine, colchicines, vinblastine, decetaxel, paclitaxel, ionizing radiation, rapamycin, rapalogs, CCI-779, RAD001, trastuzumab, and A443654.

13. The method of claim 11, additionally comprising administering to said subject one or more other anti-cancer agents.

14. A method for treating tumors or tumor metastases in a patient, comprising administering to said patient simultaneously or sequentially a synergistically effective therapeutic amount of a combination of an anti-cancer agent or treatment that elevates pAkt levels in tumor cells and an IGF-1R kinase inhibitor of Formula (I).

15. The method of claim 14, wherein the anti-cancer agent or treatment is selected from anthracyclins, doxorubicin, daunorubicin, DNA-damaging agents, cisplatin, carboplatin, topoisomerase inhibitors, camptothecin, etoposide, microtubule-directed agents, vincristine, colchicines, vinblastine, decetaxel, paclitaxel, ionizing radiation, rapamycin, rapalogs, CCI-779, RAD001, trastuzumab, and A443654.

16. The method of claim 14, additionally comprising administering to said subject one or more other anti-cancer agents.

17. The method of claim 1, wherein the cells of the tumors or tumor metastases are relatively insensitive or refractory to treatment with the anti-cancer agent or treatment as a single agent/treatment.

18. The method of claim 11, wherein the cancer is relatively insensitive or refractory to treatment with the anti-cancer agent or treatment as a single agent/treatment.

19. The method of claim 14, wherein the cells of the tumors or tumor metastases are relatively insensitive or refractory to treatment with the anti-cancer agent or treatment as a single agent/treatment.

20. A method for treating tumors or tumor metastases in a patient refractory to treatment with an anti-cancer agent or treatment that elevates pAkt levels in tumor cells as a single agent, comprising administering to said patient simultaneously or sequentially a therapeutically effective amount of a combination of said anti-cancer agent or treatment and an IGF-1R kinase inhibitor of Formula (I).

21. A pharmaceutical composition comprising an anti-cancer agent that elevates pAkt levels in tumor cells and an IGF-1R kinase inhibitor of Formula (I), in a pharmaceutically acceptable carrier.

22. The pharmaceutical composition of claim 21, wherein the anti-cancer agent is selected from anthracyclins, doxorubicin, daunorubicin, DNA-damaging agents, cisplatin, carboplatin, topoisomerase inhibitors, camptothecin, etoposide, microtubule-directed agents, vincristine, colchicines, vinblastine, decetaxel, paclitaxel, rapamycin, rapalogs, CCI-779, RAD001, trastuzumab, and A443654.

23. The pharmaceutical composition of claim 21, additionally comprising one or more other anti-cancer agents.

24. A kit comprising a container, comprising an IGF-1R kinase inhibitor of Formula (I), and an anti-cancer agent that elevates pAkt levels in tumor cells.

25. The kit of claim 24, wherein the anti-cancer agent is selected from anthracyclins, doxorubicin, daunorubicin, DNA-damaging agents, cisplatin, carboplatin, topoisomerase inhibitors, camptothecin, etoposide, microtubule-directed agents, vincristine, colchicines, vinblastine, decetaxel, paclitaxel, rapamycin, rapalogs, CCI-779, RAD 001, trastuzumab, and A443654.

26. The kit of claim 24, further comprising a sterile diluent.

27. The kit of claim 24, further comprising a package insert comprising printed instructions directing the use of a combined treatment of an IGF-1R kinase inhibitor of Formula (I) and the anti-cancer agent that elevates pAkt levels in tumor cells to a patient as a method for treating tumors, tumor metastases, or other cancers in a patient.

28. The method of claim 1, wherein the patient is in need of treatment for a cancer selected from Ewing's sarcoma, NSCL, pancreatic, head and neck, colon, ovarian and breast cancers.

29. The method of claim 11, wherein the cancer is selected from Ewing's sarcoma, NSCL, pancreatic, head and neck, colon, ovarian and breast cancers.

30. The method of claim 14, wherein the patient is in need of treatment for a cancer selected from Ewing's sarcoma, NSCL, pancreatic, head and neck, colon, ovarian and breast cancers.

31. The method of claim 20, wherein the patient is in need of treatment for a cancer selected from Ewing's sarcoma, NSCL, pancreatic, head and neck, colon, ovarian and breast cancers.

32. The method of claim 1, wherein the IGF-1R kinase inhibitor of Formula (I) comprises OSI-906.

33. The method of claim 11, wherein the IGF-1R kinase inhibitor of Formula (I) comprises OSI-906.

34. The method of claim 14, wherein the IGF-1R kinase inhibitor of Formula (I) comprises OSI-906.

35. The method of claim 20, wherein the IGF-1R kinase inhibitor of Formula (I) comprises OSI-906.

36. The composition of claim 21, wherein the IGF-1R kinase inhibitor of Formula (I) comprises OSI-906.

37. The kit of claim 24, wherein the IGF-1R kinase inhibitor of Formula (I) comprises OSI-906.

38. A method of identifying tumor cells that will respond most favorably to treatment with a combination of an anti-cancer agent or treatment that elevates pAkt levels in tumor cells and an IGF-1R kinase inhibitor, comprising:
contacting a sample of tumor cells with said anti-cancer agent or treatment that elevates pAkt levels in tumor cells,
determining whether said anti-cancer agent or treatment stimulates phosphorylation of IGF-1R or IR in the tumor cells, by comparing the level of p-IGF-1R or p-IR in tumor cells contacted with said anti-cancer agent or treatment to the level of p-IGF-1R or p-IR in an identical sample of tumor cells either not contacted with said anti-cancer agent or treatment, or contacted with a lower concentration of said anti-cancer agent or treatment, and predicting whether the sample tumor cells will respond favorably to treatment with a combination of an anti-cancer agent or treatment that elevates pAkt levels in tumor cells and an IGF-1R kinase inhibitor, wherein the higher the level of p-IGF-1R or p-IR induced by said anti-cancer agent or treatment in tumor cells, the greater likelihood that the tumor cells will respond favorably to treatment with a combination of an anti-cancer agent or treatment that elevates pAkt levels in tumor cells and an IGF-1R kinase inhibitor.

39. The method of claim 38, comprising after the step of determining whether said anti-cancer agent or treatment stimulates phosphorylation of IGF-1R or IR in the tumor cells, and before the step of predicting whether the sample tumor cells will respond favorably to treatment with a combination of an anti-cancer agent or treatment that elevates pAkt levels in tumor cells, the additional step of comparing the level of p-IGF-1R or p-IR in the sample of tumor cells contacted with said anti-cancer agent or treatment with the level of p-IGF-1R or p-IR in a control sample of tumor cells contacted with said anti-cancer agent or treatment, wherein said control sample of tumor cells is known to respond favorably to treatment with said combination of an anti-cancer agent or treatment that elevates pAkt levels in tumor cells and an IGF-1R kinase inhibitor.

40. The method of claim 38, wherein the IGF-1R kinase inhibitor comprises a anti-IGF-1R antibody or antibody fragment.

41. The method of claim 38, wherein the IGF-1R kinase inhibitor comprises a compound of Formula (I).

42. The method of claim 41, wherein the compound of Formula (I) comprises OSI-906.

43. The method of claim 38, wherein the anti-cancer agent or treatment that elevates pAkt levels in tumor cells comprises a chemotherapeutic agent or a gene-targetted anti-cancer agent.

44. The method of claim 38, wherein the anti-cancer agent or treatment that elevates pAkt levels in tumor cells is selected from anthracyclins, doxorubicin, daunorubicin, DNA-damaging agents, cisplatin, carboplatin, topoisomerase inhibitors, camptothecin, etoposide, microtubule-directed agents, vincristine, colchicines, vinblastine, decetaxel, paclitaxel, ionizing radiation, rapamycin, rapalogs, CCI-779, RAD001, trastuzumab, and A443654.

45. The method of claim 44, wherein the anti-cancer agent or treatment that elevates pAkt levels in tumor cells is doxorubicin or paclitaxel.

46. The method of claim 38, wherein the sample of tumor cells is selected from Ewing's sarcoma, NSCL, pancreatic, head and neck, colon, ovarian or breast cancer cells.

47. The method of claim 38, wherein the sample of tumor cells is a tumor or tumor biopsy from a patient with cancer.

48. The method of claim 10, wherein the anti-cancer agent or treatment that elevates pAkt levels in tumor cells is administered prior to the IGF-1R kinase inhibitor.

49. The method of claim 48, wherein the anti-cancer agent or treatment that elevates pAkt levels in tumor cells is administered at least two hours prior to the IGF-1R kinase inhibitor.

50. The method of claim 49, wherein the anti-cancer agent or treatment that elevates pAkt levels in tumor cells is administered at least four hours prior to the IGF-1R kinase inhibitor.

51. The method of claim 50, wherein the anti-cancer agent or treatment that elevates pAkt levels in tumor cells is administered at least six hours prior to the IGF-1R kinase inhibitor.

52. The method of claim 51, wherein the anti-cancer agent or treatment that elevates pAkt levels in tumor cells is administered at least twelve hours prior to the IGF-1R kinase inhibitor.

53. The method of claim 52, wherein the anti-cancer agent or treatment that elevates pAkt levels in tumor cells is administered at least twenty-four hours prior to the IGF-1R kinase inhibitor.
